(19) United States
(12) Patent Application Publication
(10) Pub. No.: US 2018/0251434 A1
(43) Pub. Date: Sep. 6, 2018

(54) ANTIBACTERIAL HETEROCYCLIC DERIVATIVES

(71) Applicant: ACTELION PHARMACEUTICALS LTD, Allschwil (CH)

(72) Inventors: Stefan DIETHELM, Allschwil (CH); Jean-Christophe GAUVIN, Allschwil (CH); Gaelle MATHIEU, Allschwil (CH); Philippe PANCHAUD, Allschwil (CH); Jean-Philippe SURIVET, Allschwil (CH)

(21) Appl. No.: 15/755,411

(22) PCT Filed: Aug. 30, 2016
(86) PCT No.: PCT/EP2016/070362

§ 371 Date: Feb. 26, 2018

(30) Foreign Application Priority Data
Aug. 31, 2015 (EP) PCT/EP2015/069837

(51) Int. Cl. C07D 263/58; C07D 277/68; A61P 31/04

(52) U.S. Cl. CPC C07D 263/58; A61P 31/04; C07D 277/68

(57) ABSTRACT

The invention relates to antibacterial compounds of formula I

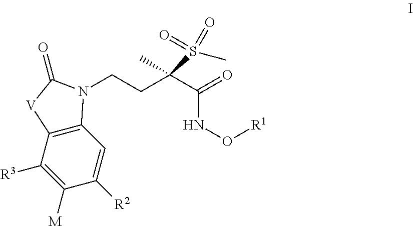

wherein R¹, M, R², R³, and V are as described in the description, to their preparation, to salts thereof, and to their use as pharmaceuticals, to pharmaceutical compositions containing one or more compounds of formula I, and especially to their use as antibacterial agents.

The present invention concerns antibacterial heterocyclic derivatives, pharmaceutical compositions containing them and uses of these compounds in the manufacture of medicaments for the treatment of bacterial infections. These compounds are useful antimicrobial agents effective against a variety of human and veterinary pathogens, especially Gram-negative aerobic and anaerobic bacteria. The compounds of the present invention can optionally be employed in combination, either sequentially or simultaneously, with one or more therapeutic agents effective against bacterial infections.

The intensive use of antibiotics has exerted a selective evolutionary pressure on microorganisms to produce genetically based resistance mechanisms. Modern medicine and socio-economic behaviour exacerbate the problem of resistance development by creating slow growth situations for pathogenic microbes, e.g. in artificial joints, and by supporting long-term host reservoirs, e.g. in immune-compromised patients.

In hospital settings, an increasing number of strains of Staphylococcus aureus, Streptococcus pneumoniae, Enterococcus spp., Enterobacteriaceae such as Klebsiella pneumoniae, Acinetobacter baumannii and Pseudomonas aeruginosa, major sources of infections, are becoming multi-drug resistant and therefore difficult if not impossible to treat. This is particularly the case for Gram-negative organisms where the situation is getting worrisome since no novel agents have been approved for decades and the development pipeline looks empty.

Therefore, there is an important medical need for new antibacterial compounds addressing Gram-negative resistant bacteria, in particular third generation cephalosporins- and carbapenem-resistant Klebsiella pneumoniae and multi-drug-resistant Pseudomonas aeruginosa and Acinetobacter baumannii. One way to tackle the problem of cross resistance to established classes of antibiotics is to inhibit a new target. In this respect, LpxC, which is an essential enzyme in the biosynthesis of lipopolysaccharides (a major constituent of the outer membrane of Gram-negative bacteria), has received some attention and several patent applications relating to LpxC inhibitors have been published recently.

For example, WO 2011/045703, WO 2011/073845, WO 2012/120397, WO 2012/137094, WO 2012/137099, WO 2013/170165 and WO 2015/066413 describe antibacterial compounds having a N-hydroxy-2-methyl-2-(methylsulfonyl)butanamide side chain bound to a monocyclic aromatic or heteroaromatic ring system.

Furthermore WO 2013/170165 describes notably antibacterial compounds of formula (A0)

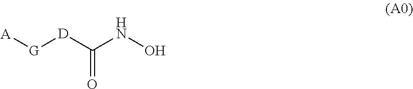

wherein A is a substituted alkyl group, wherein at least one substituent is hydroxy, or A is a substituted cycloalkyl group, wherein at least one substituent is hydroxy or hydroxyalkyl; G is a group comprising at least one carbon-carbon double or triple bond and/or a phenyl ring; D represents a group selected from

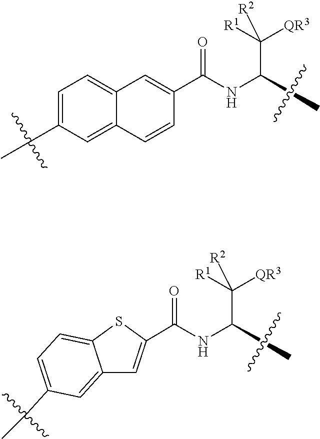

Q is O or NR, wherein R is H or an unsubstituted (C₁-C₃)alkyl; R¹ and R² independently are selected from the group consisting of H and substituted or unsubstituted (C₁-C₃)alkyl, or R¹ and R², together with the carbon atom to which they are attached, form an unsubstituted (C₃-C₄)cycloalkyl group or an unsubstituted 4-6 membered heterocyclic group; and R³ is selected from the group consisting of hydrogen, substituted or unsubstituted (C₁-C₃)alkyl, substituted or unsubstituted cycloalkyl, substituted or unsubstituted cycloalkylalkyl, substituted or unsubstituted aryl, substituted or unsubstituted arylalkyl, substituted or unsubstituted heterocyclyl, substituted or unsubstituted heterocyclylalkyl, substituted or unsubstituted heteroaryl, and substituted or unsubstituted heteroarylalkyl.

In WO 2015/036964, we have reported antibacterial 2H-indazole derivatives of general formula (A1)

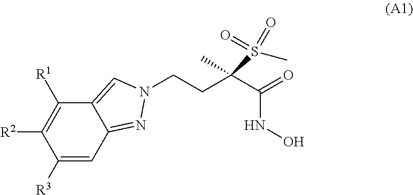

wherein

R¹ is H or halogen; R² is (C₃-C₄)alkynyloxy or the group M; R³ is H or halogen; M is one of the groups M^{A} and M^{B} represented below

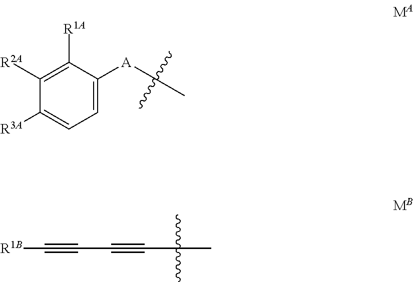

wherein A is a bond, CH₂CH₂, CH═CH or C≡C; R^{1A} represents H or halogen; R^{2A} represents H, alkoxy or halogen; R^{3A} represents H, alkoxy, hydroxyalkoxy, thioalkoxy, trifluoromethoxy, amino, dialkylamino, hydroxyalkyl, 1-hydroxymethyl-cycloprop-1-yl, trans-2-hydroxymethyl-cycloprop-1-yl, 1,2-dihydroxyethyl, 3-hydroxyoxetan-3-yl, 3-(hydroxyalkyl)oxetan-3-yl, 3-aminooxetan-3-yl, 3-(dialkylamino)oxetan-3-yl, 3-hydroxythietan-3-yl, morpholin-4-ylalkoxy, morpholin-4-ylalkyl, oxazol-2-yl or [1,2,3]triazol-2-yl; and R^{1B} represents 3-hydroxyoxetan-3-yl, 3-hydroxythietan-3-yl, hydroxyalkyl, aminoalkyl, trans-2-hydroxymethyl-cycloprop-1-yl or 4-hydroxytetrahydro-2H-pyran-4-yl.

In WO 2015/091741, we have reported antibacterial 1H-indazole derivatives of general formula (A2)

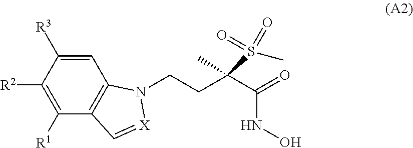

wherein

X represents N or CH;

R¹ represents H or halogen;

R² represents (C₃-C₄)alkynyloxy or the group M;

R³ represents H or halogen;

M is one of the groups M^{A} and M^{B} represented below

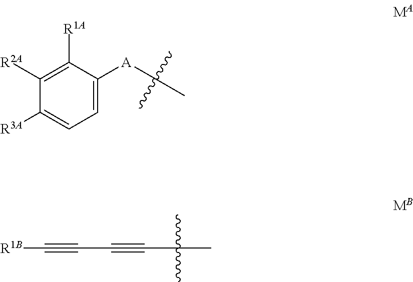

wherein A represents a bond, CH₂CH₂, CH═CH or C≡C;

R^{1A} represents H or halogen;

R^{2A} represents H, (C₁-C₃)alkoxy or halogen;

R³A represents H, (C₁-C₃)alkoxy, hydroxy(C₁-C₄)alkoxy, (C₁-C₃)thioalkoxy, trifluoromethoxy, amino, hydroxy(C₁-C₄)alkyl, 2-hydroxyacetamido, 1-hydroxymethyl-cycloprop-1-yl, trans-2-hydroxymethyl-cycloprop-1-yl, 1,2-dihydroxyethyl, 3-hydroxyoxetan-3-yl, 3-(hydroxy(C₁-C₃)alkyl)oxetan-3-yl, 3-aminooxetan-3-yl, 3-hydroxythietan-3-yl, morpholin-4-yl(C₂-C₃)alkoxy, morpholin-4-yl-(C₁-C₂)alkyl, oxazol-2-yl or [1,2,3]triazol-2-yl; and

R^{1B} represents 3-hydroxyoxetan-3-yl, 3-hydroxythietan-3-yl, hydroxy(C₁-C₃)alkyl, amino (C₁-C₃)alkyl, 1-hydroxymethyl-cycloprop-1-yl or trans-2-hydroxymethyl-cycloprop-1-yl.

In WO 2015/132228, we have reported antibacterial 1,2-dihydro-3H-pyrrolo[1,2-c]imidazol-3-one derivatives of general formula (A3)

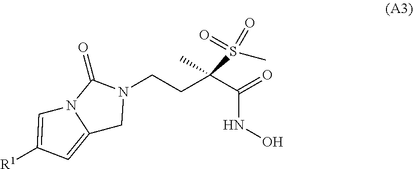

wherein R¹ is the group M; M is one of the groups M^{A} and M^{B} represented below

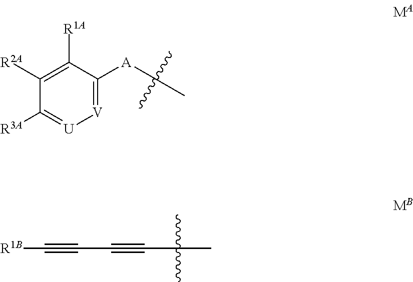

wherein A is a bond, CH═CH or C≡C; U is N or CH; V is N or CH; R^{1A} is H or halogen; R^{2A} is H, (C₁-C₃)alkoxy or halogen; R^{3A} is H, (C₁-C₃)alkoxy, hydroxy(C₂-C₄)alkoxy, (C₁-C₃)alkoxy(C₁-C₃)alkoxy, (C₁-C₃)thioalkoxy, trifluoromethoxy, amino, hydroxy(C₁-C₄)alkyl, (C₁-C₃)alkoxy(C₁-C₄)alkyl, 3-hydroxy-3-methylbut-1-yn-1-yl, 2-hydroxyacetamido, (carbamoyloxy)methyl, 1-hydroxymethyl-cycloprop-1-yl, 1-aminomethyl-cycloprop-1-yl, 1-(carbamoyloxy)methyl-cycloprop-1-yl, 1-(morpholin-4-yl)methylcycloprop-1-yl, trans-2-hydroxymethyl-cycloprop-1-yl, 1,2-dihydroxyethyl, 3-hydroxyoxetan-3-yl, 3-(hydroxy(C₁-C₃)alkyl)oxetan-3-yl, 3-aminooxetan-3-yl, 3-hydroxythietan-3-yl, morpholin-4-yl(C₂-C₃)alkoxy, [4-N—(C₁-C₃)alkylpiperazin-1-yl](C₁-C₃)alkyl, morpholin-4-yl-(C₁-C₂)alkyl, [1,2,3]triazol-2-yl or 3-[hydroxy(C₂-C₃)alkyl]-2-oxo-imidazolidin-1-yl; and R^{1B} is 3-hydroxyoxetan-3-yl, 3-hydroxythietan-3-yl, 3-(hydroxy(C₁-C₃)alkyl)oxetan-3-yl, hydroxy(C₁-C₃)alkyl, 1,2-dihydroxyethyl, amino(C₁-C₃)alkyl, 1-hydroxymethyl-cycloprop-1-yl, trans-2-hydroxymethyl-cycloprop-1-yl, trans-(cis-3,4-dihydroxy)-cyclopent-1-yl or 3-hydroxymethylbicyclo[1,1,1]pentan-1-yl.

In WO 2015/173329, we have reported antibacterial quinazoline-4(3H)-one derivatives of general formula (A4)

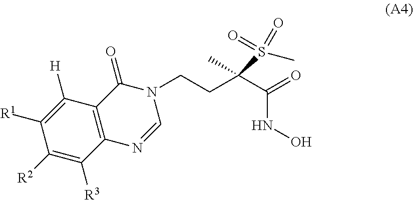

wherein R¹ is H or halogen; R² is the group M; R³ is H or halogen; M is one of the groups M_{A} and M^{B} represented below

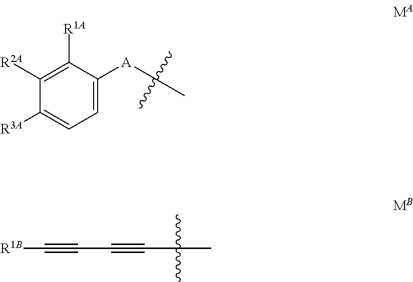

wherein A represents a bond or C≡C; R^{1A} is H or halogen; R^{2A} is H, (C₁-C₃)alkoxy or halogen; R^{3A} is H, (C₁-C₃)alkoxy, hydroxy(C₂-C₄)alkoxy, hydroxy(C₁-C₄)alkyl, 1,2-dihydroxyethyl, di(C₁-C₃)alkylamino, 1-hydroxymethyl-cycloprop-1-yl, 1-((dimethylglycyl)oxy)methyl-cycloprop-1-yl, 3-hydroxyoxetan-3-yl, morpholin-4-yl-(C₁-C₂)alkyl or morpholin-4-yl(C₂-C₃)alkoxy; and R^{1B} is hydroxy(C₁-C₃)alkyl, amino(C₁-C₃)alkyl, 1,2-dihydroxyprop-3-yl, 1-amino-cy cloprop-1-yl, 1-hydroxymethyl-cycloprop-1-yl, trans-2-hydroxymethyl-cycloprop-1-yl, trans-2-aminomethyl-cycloprop-1-yl, trans-2-hydroxymethyl-1-methyl-cycloprop-1-yl, trans-2-hydroxymethyl-2-methyl-cycloprop-1-yl, 1-(1,2-dihydroxyethyl)-cycloprop-1-yl, trans-2-(1,2-dihydroxyethyl)-cycloprop-1-yl, 3-hydroxyoxetan-3-yl, 3-(hydroxy(C₁-C₃)alkyl)oxetan-3-yl, 3-hydroxythietan-3-yl, trans-(cis-3,4-dihydroxy)-cyclopent-1-yl, 3-(2-aminoacetamido)cyclopentyl or 3-hydroxymethylbicyclo[1,1,1]pentan-1-yl.

In WO 2016/079688, we have reported antibacterial benzothiazole derivatives of general formula (A5)

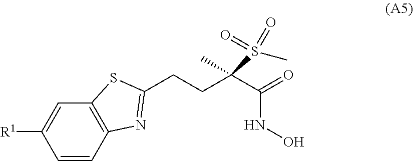

wherein

R¹ is the group M, whereby M is one of the groups M^{A} and M^{B} represented below

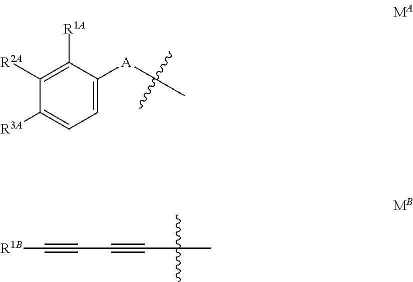

wherein A represents a bond or C≡C;

R^{1A} is H or halogen;

R^{2A} is H or halogen; and

R^{3A} is H, (C₁-C₃)alkoxy, hydroxy(C₂-C₄)alkoxy, hydroxy(C₁-C₄)alkyl, dihydroxy(C₂-C₄)alkyl, 2-hydroxyacetamido, 1-hydroxymethyl-cycloprop-1-yl, trans-2-hydroxymethyl-cycloprop-1-yl, 3-hydroxyoxetan-3-yl, 3-(hydroxy(C₁-C₃)alkyl)oxetan-3-yl, 3-aminooxetan-3-yl or 1-aminocycloprop-1-yl; and wherein R^{1B} is hydroxy(C₁-C₄)alkyl, dihydroxy(C₂-C₄)alkyl, amino(C₁-C₄)alkyl, di(C₁-C₄)alkyl amino (C₁-C₃)al kyl, 1-amino-cycloprop-1-yl, 1-hydroxymethyl-cycloprop-1-yl, trans-2-hydroxymethyl-cycloprop-1-yl, trans -2-aminomethyl-cycloprop-1-yl, trans-2-hydroxymethyl -1-methyl-cycloprop-1-yl, trans-2-hydroxymethyl-2-methyl-cycloprop-1-yl, cis-1-fluoro-2-(hydroxymethyl)cycloprop-1-yl, cis-2-fluoro-2-(hydroxymethyl)cycloprop-1-yl, 2-(1,2-dihydroxyethyl)-cycloprop-1-yl, 1-(hydroxymethyl)-cyclobutan-1-yl, cis-3-(hydroxymethyl)-1-hydroxy-cyclobutan-1-yl, 3-hydroxyoxetan-3-yl, 3-hydroxyoxetan-3-yl-(C₁-C₃)alkyl, 3-aminooxetan-3-yl, 3-hydroxymethyl-oxetan-3-yl, trans-(cis-3,4-dihydroxy)-cyclopent-1-yl, 3-hydroxymethylbicyclo[1,1,1]pentan-1-yl, 4-hydroxytetrahydro-2H-pyran-4-yl, (3R,6S)-3-aminotetrahydro-2H-pyran-6-yl, piperidin-4-yl, 1-(2-hydroxyacetyl)piperidin-4-yl, 3-hydroxythietan-3-yl, 1-(2-hydroxyacetyl)azetidin-3-yl or 1-glycylazetidin-3-yl; and salts thereof.

Besides, in Montgomery et al., J. Med. Chem. (2012), 55(4), 1662-1670, yet further LpxC inhibitors are disclosed, among others the compound of formula (A6)

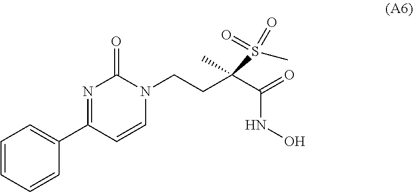 [SMILES: C[C@@](CCn1ccc(-c2ccccc2)nc1=O)(C(=O)NO)S(C)(=O)=O]

The instant invention provides new antibacterial heterocyclic derivatives, namely the compounds of formula I described herein.

1) A first embodiment of the invention relates to compounds of formula I

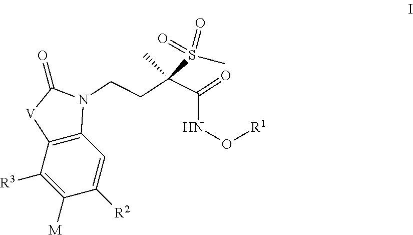

wherein

V represents O or S;

R² and R³ each independently represent hydrogen or fluorine; and

M is one of the groups M^{A} and M^{B} represented below

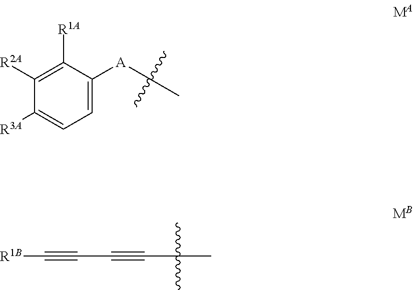

wherein in group M^{A}:
A represents a bond or —C≡C—;
R^{1A} represents hydrogen, or halogen (especially fluoro);
R^{2A} represents hydrogen, or halogen (especially fluoro); and
R^{3A} represents
   (C₁-C₃)alkoxy (especially methoxy);
   hydroxy(C₁-C₄)alkyl (especially hydroxymethyl); or
   R^{NA1}R^{NA2}N—(C₁-C₃)alkyl, wherein R^{NA1} and R^{NA2} independently represent hydrogen or (C₁-C₃)alkyl; or R^{NA1} and R^{NA2} together with the nitrogen to which they are attached form a 4- to 6-membered saturated ring optionally containing one oxygen ring atom, wherein said ring is unsubstituted, or mono-substituted with fluoro;
   [in particular such group R^{NA1}R^{NA2}N—(C₁-C₃)alkyl is amino-methyl, (C₁-C₃)alkylamino-methyl, di(C₁-C₃)alkylamino-methyl, morpholin-4-yl-(C₁-C₂)alkyl such as especially (morpholin-4-yl)-methyl, or (3-fluoroazetidin-1-yl)-methyl]; and

wherein in group M^{B}:
R^{1B} represents
   hydroxy(C₁-C₄)alkyl (especially hydroxymethyl);
   1-(3-hydroxyazetidine)-1-carbonyloxymethyl;
   1-hydroxymethyl-cycloprop-1-yl, or a group which is a prodrug thereof wherein said group is 1-((phosphonooxy)methyl)-cycloprop-1-yl, 1-[(di(C₁-C₄)alkylamino)-(C₁-C₃)alkyl-carbonyloxymethyl]-cycloprop-1-yl (especially 1-(2-dimethylaminoacetoxymethyl)-cycloprop-1-yl), 1-{[(2-(phosphonooxy-(C₁-C₄)alkyl)-phenyl)-(C₁-C₄)alkyl]-carbonyloxymethyl}-cycloprop-1-yl, 1-{[2-(phosphonooxy-(C₁-C₄)alkyl)-phenyl]-carbonyloxymethyl}-cycloprop-1-yl, or 1-{[(2-phosphonooxy-phenyl)-(C₁-C₄)alkyl]-carbonyloxymethyl}-cycloprop-1-yl;
   trans-2-hydroxymethyl-cycloprop-1-yl, or a group which is a prodrug thereof wherein said group is trans-2-(phosphonooxymethyl)-cycloprop-1-yl, trans-2-di(C₁-C₄)alkyl amino)-(C₁-C₃)alkyl-carbony loxy methyl]-cycloprop-1-yl (especially trans-2-(2-dimethylaminoacetoxymethyl)-cycloprop-1-yl), trans -2-{[(2-(phosphonooxy-(C₁-C₄)alkyl)-phenyl)-(C₁-C₄)alkyl]-carbonyloxymethyl}-cycloprop-1-yl, trans -2-{[2-(phosphonooxy-(C₁-C₄)alkyl)-phenyl]-carbonyloxymethyl}-cycloprop-1-yl, or trans-2-{[(2-phosphonooxy-phenyl)-(C₁-C₄)alkyl]-carbonyloxymethyl}-cycloprop-1-yl;
   2-fluoro-2-hydroxymethyl-cycloprop-1-yl, or a group which is a prodrug thereof wherein said group is 2-fluoro-2-(phosphonooxymethyl)-cycloprop-1-yl, 2-fluoro-2-[(di(C₁-C₄)alkylamino)-(C₁-C₃)alkyl-carbonyloxymethyl]-cycloprop-1-yl (especially 2-fluoro-2-(2-dimethylaminoacetoxymethyl)-cycloprop-1-yl), 2-fluoro-2-{[(2-(phosphonooxy-(C₁-C₄)alkyl)-phenyl)-(C₁-C₄)alkyl]-carbonyloxymethyl}-cycloprop-1-yl, 2-fluoro-2-{[2-(phosphonooxy-(C₁-C₄)alkyl)-phenyl]-carbonyloxymethyl}-cycloprop-1-yl, or 2-fluoro-2-{[(2-phosphonooxy-phenyl)-(C₁-C₄)alkyl]-carbonyloxymethyl}-cycloprop-1-yl;
   3-hydroxy-oxetan-3-yl;
   trans-2-(1,2-dihydroxyethyl)cycloprop-1-yl;
   trans-(cis-3,4-dihydroxy)-cyclopent-1-yl;
   R^{NB1}R^{NB2}N—(C₁-C₃)alkyl, wherein R^{NB1} and R^{NB2} independently represent hydrogen or (C₁-C₃)alkyl (especially methyl);
   [in particular such group R^{NB1}R^{NB2}N—(C₁-C₃)alkyl is selected from aminomethyl, (C₁-C₃)alkylamino-methyl, and di(C₁-C₃)alkylaminomethyl];
   1-(R^{NB3}R^{NB4}N)-cycloprop-1-yl, wherein R^{NB3} and R^{NB4} independently represent hydrogen or (C₁-C₃)alkyl (especially methyl), or R^{NB3} represents hydrogen and R^{NB4} (phosphonooxy)methoxy-carbonyl (wherein it is understood that such group 1-{[(phosphonooxy)methoxy-carbonyl]-amino}-cycloprop-1-yl represents a prodrug of 1-amino-cycloprop-1-yl);
   [in particular such group 1-(R^{NB3}R^{NB4}N)-cycloprop-1-yl is 1-aminocycloprop-1-yl, or a group which is a prodrug thereof wherein said group is 1-((((phosphonooxy)methoxy)carbonyl)amino)-cycloprop-1-yl; 1-(methylamino)cycloprop-1-yl; or 1-(dimethylamino)cycloprop-1-yl];
   azetidin-3-yl, wherein said azetidin-3-yl is unsubstituted, or mono- or di-substituted wherein, if present,
      one substituent is attached in position 1 of said azetidin-3-yl group, wherein said substituent is selected from (C₁-C₃)alkyl (especially methyl, ethyl, isopropyl), (C₃-C₄)cycloalkyl (especially cyclopropyl), oxetan-3-yl, ω-fluoro-(C₂)alkyl (especially 2-fluoroethyl), or ω-hydroxy-(C₂-C₄)alkyl (especially 2-hydroxy-ethyl); and/or
      one substituent is fluoro attached in position 3 of said the azetidin-3-yl group;
   [in particular such azetidin-3-yl group is azetidin-3-yl, N-methylazetidin-3-yl, N-ethylazetidin-3-yl, N-isopropylazetidin-3-yl, N-cyclopropylazetidin-3-yl, 1-(oxetan-3-yl)-azetidin-3-yl, N-(2-fluoroethyl)azetidin-3-yl, N-(2-hydroxyethyl)azetidin-3-yl, 3-fluoroazetidin-3-yl, 3-fluoro-1-methyl-azetidin-3-yl, 3-fluoro-1-ethyl-azetidin-3-yl, 3-fluoro-1-isopropyl-azetidin-3-yl, 3-fluoro-1-cyclopropyl-azetidin-3-yl, or 3-fluoro-1-(oxetan-3-yl)azetidin-3-yl];

R¹ represents hydrogen, or R¹ represents —PO₃H₂, —SO₃H, phosphonooxymethyl, or the group L represented below

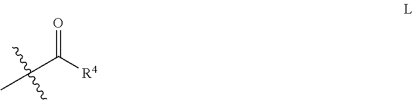

wherein R⁴ represents (C₁-C₄)alkylamino(C₁-C₄)alkyl (especially methylaminomethyl), di(C₁-C₄)alkylamino(C₁-C₄)alkyl (especially dimethylaminomethyl), phosphonooxy(C₁-C₄)alkyl (especially phosphonooxymethyl), phosphonooxymethoxy, 2-(phosphonooxy-(C₁-C₄)alkyl)-phenyl, [2-(phosphonooxy-(C₁-C₄)alkyl)-phenyl]-(C₁-C₄)alkyl, or (2-(phosphonooxy)-phenyl)-(C₁-C₄)alkyl (especially 2-(2-(phosphonooxy)-phenyl)-ethyl) (wherein it is understood that in case R¹ is different from hydrogen, such groups R¹ represent prodrugs of the N-hydroxycarboxamide moiety).

The following paragraphs provide definitions of the various chemical moieties for the compounds according to the invention and are intended to apply uniformly throughout the specification and claims, unless an otherwise expressly set out definition provides a broader or narrower definition:

The term “alkyl”, used alone or in combination, refers to a straight or branched chain saturated hydrocarbon group containing from one to six carbon atoms. The term “(Cₓ-C_{y})alkyl” (x and y each being an integer) refers to a straight or branched chain alkyl group containing x to y carbon atoms. For example, a (C₁-C₃)alkyl group contains from one to three carbon atoms. Representative examples of alkyl groups include methyl, ethyl, propyl, iso-propyl, n-butyl, iso-butyl, sec-butyl and tert-butyl. Preferred are methyl and ethyl. Most preferred is methyl.

The term “cycloalkyl”, used alone or in combination, refers to a saturated monocyclic hydrocarbon ring containing three to six carbon atoms. The term “(Cₓ-C_{y})cycloalkyl” (x and y each being an integer), refers to a cycloalkyl group as defined before containing x to y carbon atoms. For example a (C₃-C₄)cycloalkyl group contains from three to four carbon atoms. Examples of cycloalkyl groups are cyclopropyl, cyclobutyl, cyclopentyl, and cyclohexyl. Examples of (C₃-C₄)cycloalkyl groups are cyclopropyl and cyclobutyl, especially cyclopropyl.

The term “fluoroalkyl” refers to an alkyl group as defined before containing one to three carbon atoms in which one or more (and possibly all) hydrogen atoms have been replaced with fluorine. The term “(Cₓ-C_{y})fluoroalkyl” (x and y each being an integer) refers to a fluoroalkyl group as defined before containing x to y carbon atoms. An “ω-(C₂-C₃)fluoroalkyl” group refers to an alkyl group of two or three carbon atoms in which one, two or three terminal hydrogen atoms have been replaced with fluorine. Representative examples of fluoroalkyl groups include especially the ω-(C₂)fluoroalkyl groups 2-fluoroethyl, 2,2-difluoroethyl and 2,2,2-trifluoroethyl. Preferred ω-(C₂)fluoroalkyl groups as used for substituent R^{1B} are 2-fluoroethyl and 2,2,2-trifluoroethyl, especially 2-fluoroethyl.

The term “hydroxyalkyl”, used alone or in combination, refers to an alkyl group as defined before wherein one hydrogen atom has been replaced by a hydroxy group. The term “hydroxy(Cₓ-C_{y})alkyl” (x and y each being an integer) refers to a hydroxyalkyl group as defined which contains x to y carbon atoms. For example, a hydroxy(C₁-C₃)alkyl group is a hydroxyalkyl group as defined before which contains from one to three carbon atoms. Representative examples of hydroxyalkyl groups are hydroxy(C₁-C₃)alkyl groups such as hydroxymethyl, 2-hydroxyethyl, 2-hydroxypropyl and 3-hydroxypropyl. Preferred are hydroxymethyl and 2-hydroxyethyl. Most preferred is hydroxymethyl. An “ω-hydroxy(C₂-C₄)alkyl” group as used for substituent R^{1B} is a linear alkyl group which contains from two to four carbon atoms in which one terminal hydrogen atom has been replaced with hydroxy. Preferred examples of ω-hydroxy(C₂-C₄)alkyl groups as used for substituent R^{1B} are the ω-hydroxy(C₂-C₃)alkyl groups 2-hydroxyethyl and 3-hydroxypropyl, especially 2-hydroxyethyl.

It is understood that groups —O—R¹ in the fragment CO—NH—O—R¹ wherein R¹ is not H, or derivatives of a hydroxy group when present in a group M, such as phosphonooxy, (di(C₁-C₄)alkylamino)-(C₁-C₃)alkyl-carbonyloxy (e.g. dimethylaminoacetoxy), [(2-(phosphonooxy-(C₁-C₄)alkyl)-phenyl)-(C₁-C₄)alkyl]-carbonyloxy, [2-(phosphonooxy-(C₁-C₄)alkyl)-phenyl]-carbonyloxy, or [(2-phosphonooxy-phenyl)-(C₁-C₄)alkyl]-carbonyloxy (e.g. [2-(2-phosphonooxy-phenyl)-ethyl]-carbonyloxy) represent prodrugs of the corresponding —CO—NH—OH group, respectively, the corresponding hydroxy group. The term prodrug in the context of a —CO—NH—OH or hydroxy group, thus preferably refers to the above-mentioned groups [and for the —CO—NH—OH group in addition to —O—SO₃H, and —O—CH₂—O—PO₃H₂ (phosphonooxymethyl-oxy)]; in particular the term refers to phosphonooxy.

In particular,
the prodrug group (di(C₁-C₄)alkylamino)-(C₁-C₃)alkyl-carbonyloxy notably refers to dimethylaminoacetoxy;
the prodrug group [2-(phosphonooxy-(C₁-C₄)alkyl)-phenyl]-carbonyloxy notably refers to:

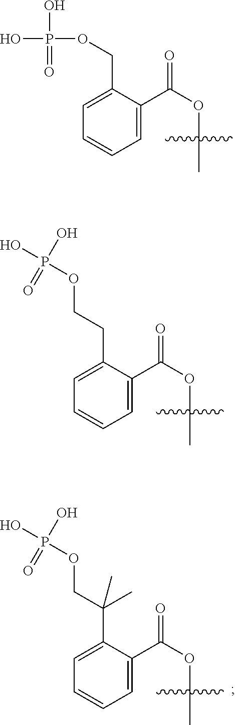 [SMILES: *OC(=O)c1ccccc1COP(=O)(O)O ; *OC(=O)c1ccccc1CCOP(=O)(O)O ; *OC(=O)c1ccccc1C(C)(C)COP(=O)(O)O]

the prodrug group [(2-(phosphonooxy-(C₁-C₄)alkyl)-phenyl)-(C₁-C₄)alkyl]-carbonyloxy notably refers to:

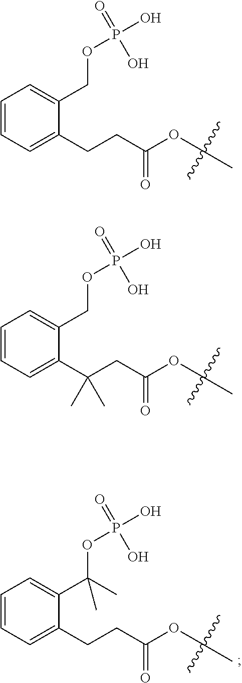 [SMILES: *OC(=O)CCc1ccccc1COP(=O)(O)O ; *OC(=O)CC(C)(C)c1ccccc1COP(=O)(O)O ; *OC(=O)CCc1ccccc1C(C)(C)OP(=O)(O)O]

the prodrug group [(2-phosphonooxy-phenyl)-(C₁-C₄)alkyl]-carbonyloxy notably refers to:

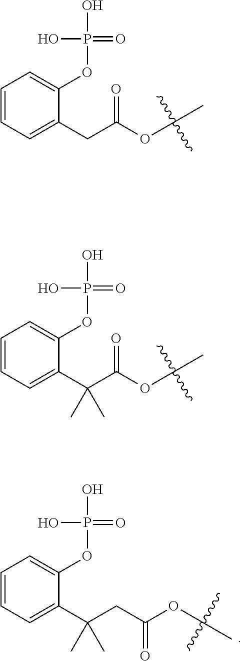 [SMILES: *OC(=O)Cc1ccccc1OP(=O)(O)O ; *OC(=O)C(C)(C)c1ccccc1OP(=O)(O)O ; *OC(=O)CC(C)(C)c1ccccc1OP(=O)(O)O]

The term “aminoalkyl”, used alone or in combination, refers to an alkyl group as defined before wherein one hydrogen atom has been replaced by an amino (—NH₂) group. The term “amino(Cₓ-C_{y})alkyl” (x and y each being an integer) refers to an aminoalkyl group as defined which contains x to y carbon atoms. For example, an amino(C₁-C₄)alkyl group is an aminoalkyl group as defined before which contains from one to four carbon atoms. Representative examples of aminoalkyl groups include aminomethyl, 2-aminoethyl, 2-aminopropyl, 2-aminoprop-2-yl and 3-aminopropyl.

The term “alkylamino”, used alone or in combination, refers to an amino group wherein one hydrogen atom has been replaced by an alkyl group as defined before. The term “(Cₓ-C_{y})alkylamino” (x and y each being an integer) refers to an alkylamino group as defined before wherein the alkyl group contains x to y carbon atoms. Representative examples of dialkylamino groups include methylamino, ethylamino, and propylamino. Preferred are methylamino and ethylamino. Most preferred is methylamino.

The term “dialkylamino”, used alone or in combination, refers to an amino group wherein each hydrogen atom has been replaced by an alkyl group as defined before, whereby the alkyl groups may be the same or different. The term “di(Cₓ-C_{y})alkylamino” (x and y each being an integer) refers to a dialkylamino group as defined before wherein each alkyl group independently contains x to y carbon atoms. For example, a di(C₁-C₄)alkylamino group is a dialkylamino group as defined before wherein each alkyl group independently contains from one to four carbon atoms. Representative examples of dialkylamino groups include dimethylamino, diethylamino, N-ethyl-N-methyl-amino and N-iso-propyl-N-methyl-amino. Preferred are dimethylamino and diethylamino. Most preferred is dimethylamino.

The term “alkylaminoalkyl”, used alone or in combination, refers to an alkylamino group as defined before wherein said alkylamino group is linked to the rest of the molecule through an alkyl group as defined before. The term “(C_{x′}-C_{y′})alkylamino(Cₓ-C_{y})alkyl” (x′, x, y′ and y each being an integer) refers to an alkylaminoalkyl group as defined which contains x′ to y′ and, respectively, x to y carbon atoms. Representative examples of alkylaminoalkyl groups are (C₁-C₄)alkylamino-(C₁-C₄)alkyl groups such as methylaminomethyl, 2-methylamino-ethyl, 2-methylaminoethan-2-yl, 3-methylamino-propyl, ethyl aminomethyl, 2-ethylamino-ethyl, 3-ethylamino-propyl, and n-propylaminomethyl; preferred is methylaminomethyl.

The term “dialkylaminoalkyl”, used alone or in combination, refers to a dialkylamino group as defined before wherein said dialkylamino group is linked to the rest of the molecule through an alkyl group as defined before. The term “di(C_{x′}-C_{y′})alkylamino(Cₓ-C_{y})alkyl” (x′, x, y′ and y each being an integer) refers to a dialkylaminoalkyl group as defined which contains x′ to y′ and, respectively, x to y carbon atoms wherein the two (C_{x′}-C_{y′})alkyl groups may be the same or different. Representative examples of dialkylaminoalkyl groups are [di(C₁-C₄)alkylamino]-(C₁-C₄)alkyl groups such as dimethylaminomethyl, 2-(dimethylamino)-ethyl, 3-(dimethylamino)-propyl, diethylaminomethyl, 2-(diethylamino)-ethyl, 3-(diethylamino)-propyl, di(n-propyl)aminomethyl, 2-(di(n-propyl)amino)-ethyl and 3-(di(n-propyl)amino)-propyl; preferred is dimethylaminomethyl.

The term “alkoxy”, used alone or in combination, refers to a straight or branched chain alkoxy group containing from one to four carbon atoms. The term “(Cₓ-C_{y})alkoxy” (x and y each being an integer) refers to an alkoxy group as defined before containing x to y carbon atoms. For example, a (C₁-C₃)alkoxy group contains from one to three carbon atoms. Representative examples of alkoxy groups include methoxy, ethoxy, n-propoxy and iso-propoxy. Preferred are methoxy and ethoxy. Most preferred is methoxy.

Examples of groups R^{NA1}R^{NA2}N—(C₁-C₃)alkyl, wherein R^{NA1} and R^{NA2} together with the nitrogen to which they are attached form a 4- to 6-membered saturated ring optionally containing one oxygen ring atom, wherein said ring is unsubstituted, or mono-substituted with fluoro” are especially azetidin-1-yl, morpholin-4-yl, and 3-fluoroazetidin-1-yl.

The term “morpholin-4-yl-(C₁-C₂)alkyl” refers to a (C₁-C₂)alkyl group as defined before wherein one of the hydrogen atoms has been replaced by a morpholin-4-yl group. Examples of morpholin-4-yl-(C₁-C₂)alkyl groups are (morpholin-4-yl)-methyl and (2-morpholin-4-yl)-ethyl. The most preferred morpholino(C₁-C₂)alkyl group is (morpholin-4-yl)-methyl. For avoidance of any doubt, in case the term morpholino is used said term refers to the morpholin-4-yl group.

The term “halogen” refers to fluorine, chlorine, bromine or iodine, and preferably to fluorine or chlorine, and most preferably to fluorine.

The term “quinolone-resistant”, when used in this text, refers to a bacterial strain against which ciprofloxacin has a Minimal Inhibitory Concentration of 16 mg/L or higher (said Minimal Inhibitory Concentration being measured with the standard method described in “Methods for Dilution Antimicrobial Susceptibility Tests for Bacteria that Grow Aerobically”, Approved standard, 7th ed., Clinical and Laboratory Standards Institute (CLSI) Document M7-A7, Wayne, Pa., USA (2006)).

The term “carbapenem-resistant”, when used in this text, refers to a bacterial strain against which imipenem has a Minimal Inhibitory Concentration of 16 mg/L or higher (said Minimal Inhibitory Concentration being measured with the standard method described in “Methods for Dilution Antimicrobial Susceptibility Tests for Bacteria that Grow Aerobically”, Approved standard, 7th ed., Clinical and Laboratory Standards Institute (CLSI) Document M7-A7, Wayne, Pa., USA (2006)).

The term “multi-drug resistant”, when used in this text, refers to a bacterial strain against which at least three antibiotic compounds selected from three distinct antibiotic categories have Minimal Inhibitory Concentrations (MICs) over their respective clinical breakpoints, whereby said three distinct antibiotic categories are chosen among penicillins, combinations of penicillins with beta-lactamase inhibitors, cephalosporins, carbapenems, monobactams, fluoro quinolones, aminoglycosides, phosphonic acids, tetracyclins and polymixins. Clinical breakpoints are defined according to the latest available list published by Clinical and Laboratory Standards Institute (Wayne, Pa., USA). Accordingly, clinical breakpoints are the levels of MIC at which, at a given time, a bacterium is deemed either susceptible or resistant to treatment by the corresponding antibiotic or antibiotic combination.

The present invention also relates to pharmacologically acceptable salts and to compositions and formulations of compounds of formula I or I_{CE}.

Any reference to a compound of formula I or I_{CE} in this text is to be understood as referring also to the salts (and especially the pharmaceutically acceptable salts) of such compounds, as appropriate and expedient.

The term “pharmaceutically acceptable salts” refers to salts that retain the desired biological activity of the subject compound and exhibit minimal undesired toxicological effects. Such salts include inorganic or organic acid and/or base addition salts depending on the presence of basic and/or acidic groups in the subject compound. For reference see for example ‘Handbook of Pharmaceutical Salts. Properties, Selection and Use.’, P. Heinrich Stahl, Camille G. Wermuth (Eds.), Wiley-VCH (2008) and ‘Pharmaceutical Salts and Co-crystals’, Johan Wouters and Luc Quéré (Eds.), RSC Publishing (2012).

In this text, a bond interrupted by a wavy line shows a point of attachment of the radical drawn to the rest of the molecule. For example, the radical drawn below

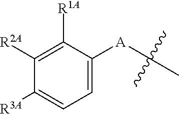

wherein A represents a bond, and each of R^{1A}, R^{2A} represents H and R^{3A} is hydroxymethyl, is a (4-hydroxymethyl)phenyl group.

In the text, it is understood that compounds of formula I wherein V represents O are benzo[d]oxazol-2(3H)-one derivatives and that compounds of formula I wherein V represents S are benzo[d]thiazol-2(3H)-one derivatives of the formulae indicated below:

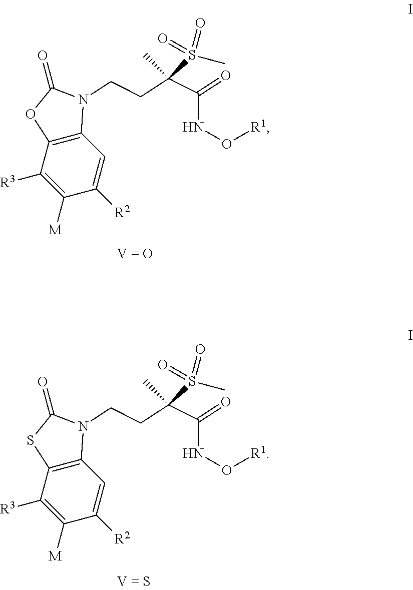

In case the relative configuration of a radical is given, it is understood that one of the chiral carbon atoms to which the indication of the relative configuration refers is the carbon atom that is attached to the rest of the molecule: e.g. trans-2-hydroxymethyl-cycloprop-1-yl refers to the radicals

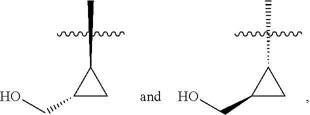

or any mixture thereof.

Likewise, cis-2-fluoro-2 hydroxymethyl-cycloprop-1-yl refers to the radicals

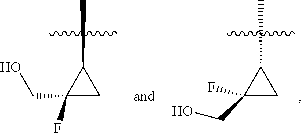

or any mixture thereof.

A 2-fluoro-2-hydroxymethyl-cycloprop-1-yl group or the correpsonding prodrug thereof, wherever used in the claims or the description, is preferably a cis-2-fluoro-2-hydroxymethyl-cycloprop-1-yl group or the corresponding prodrug thereof.

Various embodiments of the invention are presented hereafter:

2) A further embodiment relates to compounds according to embodiment 1), wherein V represents O.

3) A further embodiment relates to compounds according to embodiment 1), wherein V represents S;

4) A further embodiment relates to compounds according to any one of embodiments 1) to 3), wherein R² and R³ both represent hydrogen.

5) A further embodiment relates to compounds according to any one of embodiments 1) to 4), wherein R¹ represents hydrogen, or R¹ represents —PO₃H₂, —SO₃H, phosphonooxymethyl, or the group L represented below

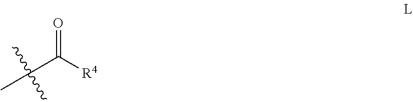

wherein R⁴ represents di(C₁-C₄)alkylamino(C₁-C₄)alkyl (especially dimethylaminomethyl) (wherein it is understood that in case R¹ is different from hydrogen, such groups R¹ represent prodrugs of the N-hydroxycarboxamide moiety).

6) A further embodiment relates to compounds according to any one of embodiments 1) to 4), wherein R¹ represents hydrogen, or R¹ represents —PO₃H₂, —SO₃H, phosphonooxymethyl, or dimethylaminomethylcarbonyl (wherein it is understood that in case R¹ is different from hydrogen, such groups R¹ represent prodrugs of the N-hydroxycarboxamide moiety).

7) A further embodiment relates to compounds according to any one of embodiments 1) to 4), wherein R¹ represents hydrogen.

8) A further embodiment relates to compounds according to any one of embodiments 1) to 7), wherein M is one of the groups M^{A} and M^{B} represented below

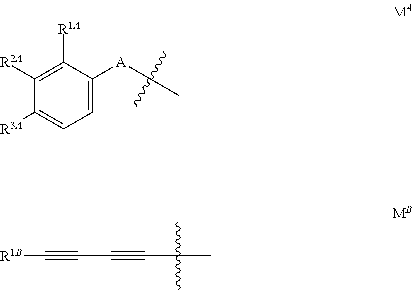

wherein in group M^{A}:
A represents a bond;
R^{1A} represents hydrogen or fluoro;
R^{2A} represents hydrogen; and
R^{3A} represents (C₁-C₃)alkoxy (especially methoxy); or
A represents —C≡C—;
R^{1A} represents hydrogen;
R^{2A} represents hydrogen; and
R^{3A} represents
   hydroxy(C₁-C₄)alkyl (especially hydroxymethyl); or
   R^{NA1}R^{NA2}N—(C₁-C₃)alkyl, wherein R^{NA1} and R^{NA2} independently represent hydrogen or (C₁-C₃)alkyl (especially methyl); or R^{NA1} and R^{NA2} together with the nitrogen to which they are attached form a 4- to 6-membered saturated ring optionally containing one oxygen ring atom, wherein said ring is unsubstituted, or mono-substituted with fluoro;
   [in particular such group R^{NA1}R^{NA2}N—(C₁-C₃)alkyl is amino-methyl, methylamino-methyl, dimethylamino-methyl, (morpholin-4-yl)-methyl, or (3-fluoroazetidin-1-yl)-methyl]; and

wherein in group M^{B}:
R^{1B} represents
   hydroxy(C₁-C₄)alkyl (especially hydroxymethyl);
   1-(3-hydroxyazetidine)-1-carbonyloxymethyl;
   1-hydroxymethyl-cycloprop-1-yl, or a group which is a prodrug thereof wherein especially said group is 1-((phosphonooxy)methyl)-cycloprop-1-yl, or 1-[(di(C₁-C₄)alkylamino)-(C₁-C₃)alkyl-carbonyloxymethyl]-cycloprop-1-yl (especially 1-(2-dimethylaminoacetoxymethyl)-cycloprop-1-yl);
   trans-2-hydroxymethyl-cycloprop-1-yl, or a group which is a prodrug thereof wherein especially said group is trans-2-(phosphonooxymethyl)-cycloprop-1-yl, or trans-2-[di(C₁-C₄)alkylamino)-(C₁-C₃)alkyl-carbonyloxymethyl]-cycloprop-1-yl (especially trans-2-(2-dimethylaminoacetoxymethyl)-cycloprop-1-yl);
   2-fluoro-2-hydroxymethyl-cycloprop-1-yl, or a group which is a prodrug thereof wherein especially said group is 2-fluoro-2-(phosphonooxymethyl)-cycloprop-1-yl;
   3-hydroxy-oxetan-3-yl;
   trans-2-(1,2-dihydroxyethyl)cycloprop-1-yl;
   trans-(cis-3,4-dihydroxy)-cyclopent-1-yl;
   R^{NB1}R^{NB2}N—(C₁-C₃)alkyl, wherein R^{NB1} and R^{NB2} independently represent hydrogen or (C₁-C₃)alkyl (especially methyl);
   [in particular such group R^{NB1}R^{NB2}N—(C₁-C₃)alkyl is selected from aminomethyl, methylamino-methyl, and dimethylaminomethyl];
   1-(R^{NB3}R^{NB4}N)-cycloprop-1-yl, wherein R^{NB3} and R^{NB4} independently represent hydrogen or (C₁-C₃)alkyl (especially methyl), or R^{NB3} represents hydrogen and R^{NB4} (phosphonooxy)methoxy-carbonyl (wherein it is understood that such group 1-{[(phosphonooxy)methoxy-carbonyl]-amino}-cycloprop-1-yl represents a prodrug of 1-amino-cycloprop-1-yl);
   [in particular such group 1-(R^{NB3}R^{NB4}N)-cycloprop-1-yl is 1-aminocycloprop-1-yl, or a group which is a prodrug thereof wherein said group is 1-((((phosphonooxy)methoxy)carbonyl)amino)-cycloprop-1-yl;
   1-(methylamino)cycloprop-1-yl; or 1-(dimethylamino)cycloprop-1-yl];
   azetidin-3-yl, wherein said azetidin-3-yl is mono-or di-substituted wherein
      one substituent is attached in position 1 of said azetidin-3-yl group, wherein said substituent is selected from (C₁-C₃)alkyl (especially methyl, ethyl, isopropyl), (C₃-C₄)cycloalkyl (especially cyclopropyl), oxetan-3-yl, ω-fluoro-(C₂)alkyl (especially 2-fluoroethyl), or ω-hydroxy-(C₂-C₄)alkyl (especially 2-hydroxy-ethyl); and, if present,
      one substituent is fluoro attached in position 3 of said the azetidin-3-yl group;
   [in particular such azetidin-3-yl group is N-methylazetidin-3-yl, N-ethylazetidin-3-yl, N-isopropylazetidin-3-yl, N-cyclopropylazetidin-3-yl, 1-(oxetan-3-yl)-azetidin-3-yl, N-(2-fluoroethyl)azetidin-3-yl, N-(2-hydroxyethyl)azetidin-3-yl, 3-fluoro-1-methyl-azetidin-3-yl, 3-fluoro-1-ethyl-azetidin-3-yl, 3-fluoro-1-isopropyl-azetidin-3-yl, 3-fluoro-1-cyclopropyl-azetidin-3-yl, or 3-fluoro-1-(oxetan-3-yl)azetidin-3-yl].

9) A further embodiment relates to compounds according to any one of embodiments 1) to 7), wherein M is one of the groups M^{A} and M^{B} represented below

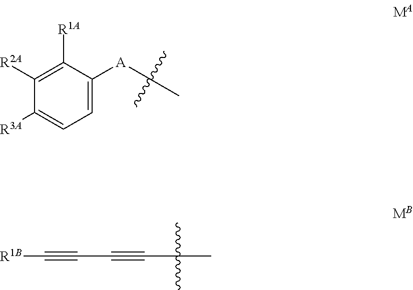

wherein in group M^{A}:
A represents a bond;
R^{1A} represents hydrogen or fluoro;
R^{2A} represents hydrogen; and
R^{3A} represents methoxy; or
A represents —C≡C—;
R^{1A} represents hydrogen;
R^{2A} represents hydrogen; and
R^{3A} represents
   hydroxymethyl; or
   amino-methyl, methylamino-methyl, dimethylamino-methyl, (morpholin-4-yl)-methyl, or (3-fluoroazetidin-1-yl)-methyl; and

wherein in group M^{B}:
R^{1B} represents
   hydroxymethyl;
   1- (3-hydroxyazetidine)-1-carbonyloxymethyl;
   1-hydroxymethyl-cycloprop-1-yl, or a group which is a prodrug thereof wherein especially said group is 1-((phosphonooxy)methyl)-cycloprop-1-yl or 1-(2-dimethylaminoacetoxymethyl)-cycloprop-1-yl ;
   trans-2-hydroxymethyl-cycloprop-1-yl, or a group which is a prodrug thereof wherein especially said group is trans-2-(phosphonooxymethyl)-cycloprop-1-yl or trans-2-(2-dimethylaminoacetoxymethyl)-cycloprop-1-yl;
   cis-2-fluoro-2-hydroxymethyl-cycloprop-1-yl, or a group which is a prodrug thereof wherein especially said group is cis-2-fluoro-2-(phosphonooxymethyl)-cycloprop-1-yl;
   3-hydroxy-oxetan-3-yl;
   trans-2-(1,2-dihydroxyethyl)cycloprop-1-yl;
   trans-(cis-3,4-dihydroxy)-cyclopent-1-yl;
   aminomethyl, methylamino-methyl, or dimethylaminomethyl;
   1-aminocycloprop-1-yl, or a group which is a prodrug thereof wherein said group is 1-((((phosphonooxy)methoxy)carbonyl)amino)-cycloprop-1-yl; 1-(methylamino)cycloprop-1-yl; or 1-(dimethylamino)cycloprop-1-yl;
   azetidin-3-yl, N-methylazetidin-3-yl, N-ethylazetidin-3-yl, N-isopropylazetidin-3-yl, N-cyclopropylazetidin-3-yl, 1-(oxetan-3-yl)-azetidin-3-yl, N-(2-fluoroethyl)azetidin-3-yl, N-(2-hydroxyethyl)azetidin-3-yl, 3-fluoroazetidin-3-yl, 3-fluoro-1-methyl-azetidin-3-yl, 3-fluoro-1-ethyl-azetidin-3-yl, 3-fluoro-1-isopropyl-azetidin-3-yl, 3-fluoro-1-cyclopropyl-azetidin-3-yl, or 3-fluoro-1-(oxetan-3-yl)azetidin-3-yl.

10) A further embodiment relates to compounds according to any one of embodiments 1) to 7), wherein M is one of the groups M^{A} and M^{B} represented below

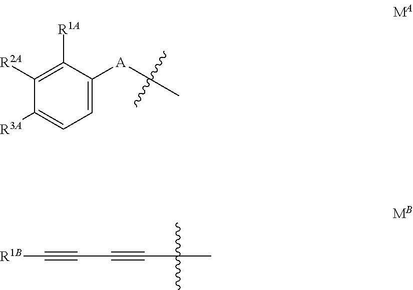

wherein in group M^{A}:
A represents a bond;
R^{1A} represents hydrogen or fluoro;
R^{2A} represents hydrogen; and
R^{3A} represents methoxy; or
A represents —C≡C—;
R^{1A} represents hydrogen;
R^{2A} represents hydrogen; and
R^{3A} represents
   hydroxymethyl; or
   (morpholin-4-yl)-methyl; and

wherein in group M^{B}:
R^{1B} represents
   1-(3-hydroxyazetidine)-1-carbonyloxymethyl;
   1-hydroxymethyl-cycloprop-1-yl, or a group which is a prodrug thereof wherein said group is especially 1-((phosphonooxy)methyl)-cycloprop-1-yl;
   trans-2-hydroxymethyl-cycloprop-1-yl, or a group which is a prodrug thereof wherein especially said group is trans-2-(phosphonooxymethyl)-cycloprop-1-yl;
   cis-2-fluoro-2-hydroxymethyl-cycloprop-1-yl, or a group which is a prodrug thereof wherein especially said group is cis-2-fluoro-2-(phosphonooxymethyl)-cycloprop-1-yl;
   3-hydroxy-oxetan-3-yl;
   1-aminocycloprop-1-yl or 1-(methylamino)cycloprop-1-yl;
   N-methylazetidin-3-yl, N-ethylazetidin-3-yl, N-isopropylazetidin-3-yl, N-cyclopropylazetidin-3-yl, 3-fluoro-1-methyl-azetidin-3-yl, 3-fluoro-1-ethyl-azetidin-3-yl, 3-fluoro-1-isopropyl-azetidin-3-yl, or 3-fluoro-1-cyclopropyl-azetidin-3-yl (especially N-methylazetidin-3-yl).

11) A further embodiment relates to compounds according to any one of embodiments 1) to 7), wherein M is one of the groups M^{A} and M^{B} represented below

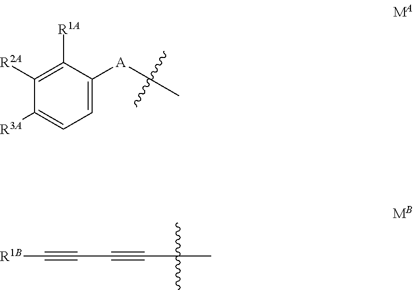

wherein in group M^{A}:
A represents —C≡C—;
R^{1A} represents hydrogen;
R^{2A} represents hydrogen; and
R^{3A} represents hydroxymethyl; and

wherein in group M^{B}:
R^{1B} represents
   1-(3-hydroxyazetidine)-1-carbonyloxymethyl;
   1-hydroxymethyl-cycloprop-1-yl;
   trans-2-hydroxymethyl-cycloprop-1-yl, or a group which is a prodrug thereof wherein especially said group is trans-2-(phosphonooxymethyl)-cycloprop-1-yl;
   cis-2-fluoro-2-hydroxymethyl-cycloprop-1-yl;
   1-aminocycloprop-1-yl or 1-(methylamino)cycloprop-1-yl;
   N-methylazetidin-3-yl, N-ethylazetidin-3-yl, N-isopropylazetidin-3-yl, N-cyclopropylazetidin-3-yl, 3-fluoro-1-methyl-azetidin-3-yl, 3-fluoro-1-ethyl-azetidin-3-yl, 3-fluoro-1-isopropyl-azetidin-3-yl, or 3-fluoro-1-cyclopropyl-azetidin-3-yl (especially N-methylazetidin-3-yl).

12) The invention in particular relates to compounds of formula I according to embodiment 1) which are also compounds of formula I_{CE}

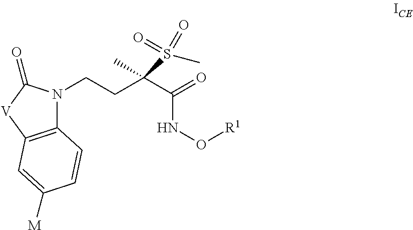

wherein

V represents O or S;

R¹ represents hydrogen;

M is one of the groups M^{A} and M^{B} represented below

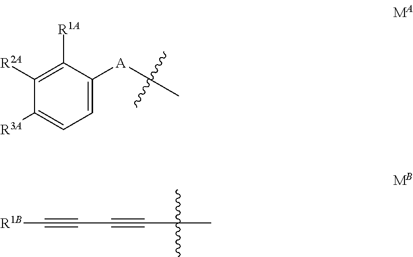

wherein in group M^{A}:
A represents a bond;
R^{1A} represents hydrogen or fluoro;
R^{2A} represents hydrogen; and
R^{3A} represents (C₁-C₃)alkoxy (especially methoxy); or
A represents —C≡C—;
R^{1A} represents hydrogen;
R^{2A} represents hydrogen; and
R^{3A} represents
   hydroxy(C₁-C₄)alkyl (especially hydroxymethyl); or
   R^{NA1}R^{NA2}N—(C₁-C₃)alkyl, wherein R^{NA1} and R^{NA2} together with the nitrogen to which they are attached form an unsubstituted morpholine ring; [in particular (morpholin-4-yl)-methyl]; and

wherein in group M^{B}:
R^{1B} represents
   hydroxy(C₁-C₄)alkyl (especially hydroxymethyl);
   1-(3-hydroxyazetidine)-1-carbonyloxymethyl;
   1-hydroxymethyl-cycloprop-1-yl, or a group which is a prodrug thereof wherein especially said group is 1-((phosphonooxy)methyl)-cycloprop-1-yl;
   trans-2-hydroxymethyl-cycloprop-1-yl, or a group which is a prodrug thereof wherein especially said group is trans-2-(phosphonooxymethyl)-cycloprop-1-yl;
   2-fluoro-2-hydroxymethyl-cycloprop-1-yl, or a group which is a prodrug thereof wherein said group is 2-fluoro-2-(phosphonooxymethyl)-cycloprop-1-yl;
   3-hydroxy-oxetan-3-yl;
   1-(R^{NB3}R^{NB4}N)-cycloprop-1-yl, wherein R^{NB3} and R^{NB4} independently represent hydrogen or (C₁-C₃)alkyl (especially methyl) [in particular such group 1-(R^{NB3RNB4}N)-cycloprop-1-yl is 1-aminocy cloprop-1-yl, or 1-(methylamino)cycloprop-1-yl];
   azetidin-3-yl, wherein said azetidin-3-yl is mono-or di-substituted wherein
      one substituent is attached in position 1 of said azetidin-3-yl group, wherein said substituent is selected from (C₁-C₃)alkyl (especially methyl, ethyl, isopropyl), (C₃-C₄)cycloalkyl (especially cyclopropyl), oxetan-3-yl, ω-fluoro-(C₂)alkyl (especially 2-fluoroethyl), or ω-hydroxy-(C₂-C₄)alkyl (especially 2-hydroxy-ethyl); and, if present,
      one substituent is fluoro attached in position 3 of said the azetidin-3-yl group;
   [in particular such azetidin-3-yl group is N-methylazetidin-3-yl, N-ethylazetidin-3-yl, N-isopropylazetidin-3-yl, N-cyclopropylazetidin-3-yl, 3-fluoro-1-methyl-azetidin-3-yl; especially it is N-methylazetidin-3-yl].

13) A further embodiment relates to compounds according to embodiment 12), wherein

V represents O or S;

R¹ represents hydrogen;

M is one of the groups M^{A} and M^{B} represented below

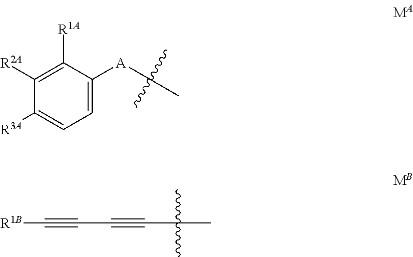

wherein in group M^{A}:
A represents a bond;
R^{1A} represents hydrogen or fluoro;
R^{2A} represents hydrogen; and
R^{3A} represents methoxy; or
A represents —C≡C—;
R^{1A} represents hydrogen;
R^{2A} represents hydrogen; and
R^{3A} represents
   hydroxymethyl; or
   (morpholin-4-yl)-methyl; and

wherein in group M^{B}:
R^{1B} represents
   1-(3-hydroxyazetidine)-1-carbonyloxymethyl;
   1-hydroxymethyl-cycloprop-1-yl;
   trans-2-hydroxymethyl-cycloprop-1-yl, or a group which is a prodrug thereof wherein especially said group is trans-2-(phosphonooxymethyl)-cycloprop-1-yl;
   2-fluoro-2-hydroxymethyl-cycloprop-1-yl;
   3-hydroxy-oxetan-3-yl;
   1-aminocycloprop-1-yl;
   1-(methylamino)cycloprop-1-yl; or
   N-methylazetidin-3-yl.

14) A further embodiment relates to compounds according to any one of embodiments 1) to 13), wherein M is the group M^{B}.

15) A further embodiment relates to compounds according to any one of embodiments 1) to 13), wherein M is the group M^{A}.

16) The invention, thus, relates to compounds of the formula I as defined in embodiment 1), and to compounds of the formula I_{CE} as defined in embodiment 12); or to such compounds further limited by the characteristics of any one of embodiments 2) to 14), under consideration of their respective dependencies; to salts and especially pharmaceutically acceptable salts thereof; and to the use of such compounds as medicaments, especially in the treatment of a bacterial infection, in particular for the prevention or treatment of a bacterial infection caused by Gram-negative bacteria (notably caused by Acinetobacter baumannii, Burkholderia spp., Citrobacter spp., Enterobacter aerogenes, Enterobacter cloacae, Escherichia coli, Klebsiella oxytoca, Klebsiella pneumoniae, Serratia marcescens, Stenotrophomonas maltophilia or Pseudomonas aeruginosa; especially caused by Acinetobacter baumannii bacteria, Escherichia coli bacteria, Klebsiella pneumoniae bacteria or Pseudomonas aeruginosa bacteria; and in particular caused by quinolone-resistant Acinetobacter baumannii bacteria or quinolone-resistant Klebsiella pneumoniae bacteria). For avoidance of any doubt, especially the following embodiments relating to the compounds of formula (I) and (I_{CE}) are thus possible and intended and herewith specifically disclosed in individualized form: 1, 2+1, 3+1, 4+1, 4+2+1, 4+3+1, 5+1, 5+2+1, 5+3+1, 5+4+1, 5+4+2+1, 5+4+3+1, 6+1, 6+2+1, 6+3+1, 6+4+1, 6+4+2+1, 6+4+3+1, 7+1, 7+2+1, 7+3+1, 7+4+1, 7+4+2+1, 7+4+3+1, 8+1, 8+2+1, 8+3+1, 8+4+1, 8+4+2+1, 8+4+3+1, 8+5+1, 8+5+2+1, 8+5+3+1, 8+5+4+1, 8+5+4+2+1, 8+5+4+3+1, 8+6+1, 8+6+2+1, 8+6+3+1, 8+6+4+1, 8+6+4+2+1, 8+6+4+3+1, 8+7+1, 8+7+2+1, 8+7+3+1, 8+7+4+1, 8+7+4+2+1, 8+7+4+3+1, 9+1, 9+2+1, 9+3+1, 9+4+1, 9+4+2+1, 9+4+3+1, 9+5+1, 9+5+2+1, 9+5+3+1, 9+5+4+1, 9+5+4+2+1, 9+5+4+3+1, 9+6+1, 9+6+2+1, 9+6+3+1, 9+6+4+1, 9+6+4+2+1, 9+6+4+3+1, 9+7+1, 9+7+2+1, 9+7+3+1, 9+7+4+1, 9+7+4+2+1, 9+7+4+3+1, 10+1, 10+2+1, 10+3+1, 10+4+1, 10+4+2+1, 10+4+3+1, 10+5+1, 10+5+2+1, 10+5+3+1, 10+5+4+1, 10+5+4+2+1, 10+5+4+3+1, 10+6+1, 10+6+2+1, 10+6+3+1, 10+6+4+1, 10+6+4+2+1, 10+6+4+3+1, 10+7+1, 10+7+2+1, 10+7+3+1, 10+7+4+1, 10+7+4+2+1, 10+7+4+3+1, 11+1, 11+2+1, 11+3+1, 11+4+1, 11+4+2+1, 11+4+3+1, 11+5+1, 11+5+2+1, 11+5+3+1, 11+5+4+1, 11+5+4+2+1, 11+5+4+3+1, 11+6+1, 11+6+2+1, 11+6+3+1, 11+6+4+1, 11+6+4+2+1, 11+6+4+3+1, 11+7+1, 11+7+2+1, 11+7+3+1, 11+7+4+1, 11+7+4+2+1, 11+7+4+3+1, 12+1, 12+2+1, 12+3+1, 13+12+1, 13+12+2+1, 13+12+3+1, 14+1, 14+8+1, 14+8+2+1, 14+8+3+1, 14+8+4+1, 14+8+4+2+1, 14+8+4+3+1, 14+8+5+1, 14+8+5+2+1, 14+8+5+3+1, 14+8+5+4+1, 14+8+5+4+2+1, 14+8+5+4+3+1, 14+8+6+1, 14+8+6+2+1, 14+8+6+3+1, 14+8+6+4+1, 14+8+6+4+2+1, 14+8+6+4+3+1, 14+8+7+1, 14+8+7+2+1, 14+8+7+3+1, 14+8+7+4+1, 14+8+7+4+2+1, 14+8+7+4+3+1, 14+9+1, 14+9+2+1, 14+9+3+1, 14+9+4+1, 14+9+4+2+1, 14+9+4+3+1, 14+9+5+1, 14+9+5+2+1, 14+9+5+3+1, 14+9+5+4+1, 14+9+5+4+2+1, 14+9+5+4+3+1, 14+9+6+1, 14+9+6+2+1, 14+9+6+3+1, 14+9+6+4+1, 14+9+6+4+2+1, 14+9+6+4+3+1, 14+9+7+1, 14+9+7+2+1, 14+9+7+3+1, 14+9+7+4+1, 14+9+7+4+2+1, 14+9+7+4+3+1, 14+10+1, 14+10+2+1, 14+10+3+1, 14+10+4+1, 14+10+4+2+1, 14+10+4+3+1, 14+10+5+1, 14+10+5+2+1, 14+10+5+3+1, 14+10+5+4+1, 14+10+5+4+2+1, 14+10+5+4+3+1, 14+10+6+1, 14+10+6+2+1, 14+10+6+3+1, 14+10+6+4+1, 14+10+6+4+2+1, 14+10+6+4+3+1, 14+10+7+1, 14+10+7+2+1, 14+10+7+3+1, 14+10+7+4+1, 14+10+7+4+2+1, 14+10+7+4+3+1, 14+11+1, 14+11+2+1, 14+11+3+1, 14+11+4+1, 14+11+4+2+1, 14+11+4+3+1, 14+11+5+1, 14+11+5+2+1, 14+11+5+3+1, 14+11+5+4+1, 14+11+5+4+2+1, 14+11+5+4+3+1, 14+11+6+1, 14+11+6+2+1, 14+11+6+3+1, 14+11+6+4+1, 14+11+6+4+2+1, 14+11+6+4+3+1, 14+11+7+1, 14+11+7+2+1, 14+11+7+3+1, 14+11+7+4+1, 14+11+7+4+2+1, 14+11+7+4+3+1, 14+12+1, 14+12+2+1, 14+12+3+1, 14+13+12+1, 14+13+12+2+1, 14+13+12+3+1, 15+1, 15+8+1, 15+8+2+1, 15+8+3+1, 15+8+4+1, 15+8+4+2+1, 15+8+4+3+1, 15+8+5+1, 15+8+5+2+1, 15+8+5+3+1, 15+8+5+4+1, 15+8+5+4+2+1, 15+8+5+4+3+1, 15+8+6+1, 15+8+6+2+1, 15+8+6+3+1, 15+8+6+4+1, 15+8+6+4+2+1, 15+8+6+4+3+1, 15+8+7+1, 15+8+7+2+1, 15+8+7+3+1, 15+8+7+4+1, 15+8+7+4+2+1, 15+8+7+4+3+1, 15+9+1, 15+9+2+1, 15+9+3+1, 15+9+4+1, 15+9+4+2+1, 15+9+4+3+1, 15+9+5+1, 15+9+5+2+1, 15+9+5+3+1, 15+9+5+4+1, 15+9+5+4+2+1, 15+9+5+4+3+1, 15+9+6+1, 15+9+6+2+1, 15+9+6+3+1, 15+9+6+4+1, 15+9+6+4+2+1, 15+9+6+4+3+1, 15+9+7+1, 15+9+7+2+1, 15+9+7+3+1, 15+9+7+4+1, 15+9+7+4+2+1, 15+9+7+4+3+1, 15+10+1, 15+10+2+1, 15+10+3+1, 15+10+4+1, 15+10+4+2+1, 15+10+4+3+1, 15+10+5+1, 15+10+5+2+1, 15+10+5+3+1, 15+10+5+4+1, 15+10+5+4+2+1, 15+10+5+4+3+1, 15+10+6+1, 15+10+6+2+1, 15+10+6+3+1, 15+10+6+4+1, 15+10+6+4+2+1, 15+10+6+4+3+1, 15+10+7+1, 15+10+7+2+1, 15+10+7+3+1, 15+10+7+4+1, 15+10+7+4+2+1, 15+10+7+4+3+1, 15+11+1, 15+11+2+1, 15+11+3+1, 15+11+4+1, 15+11+4+2+1, 15+11+4+3+1, 15+11+5+1, 15+11+5+2+1, 15+11+5+3+1, 15+11+5+4+1, 15+11+5+4+2+1, 15+11+5+4+3+1, 15+11+6+1, 15+11+6+2+1, 15+11+6+3+1, 15+11+6+4+1, 15+11+6+4+2+1, 15+11+6+4+3+1, 15+11+7+1, 15+11+7+2+1, 15+11+7+3+1, 15+11+7+4+1, 15+11+7+4+2+1, 15+11+7+4+3+1, 15+12+1, 15+12+2+1, 15+12+3+1, 15+13+12+1, 15+13+12+2+1, 15+13+12+3+1.

In the list above the numbers refer to the embodiments according to their numbering provided hereinabove whereas “+” indicates the dependency from another embodiment. The different individualized embodiments are separated by commas. In other words, “10+4+2+1” for example refers to embodiment 10) depending on embodiment 4), depending on embodiment 2), depending on embodiment 1), i.e. embodiment “10+4+2+1” corresponds to the compounds of formula I according to embodiment 1) further limited by all the features of the embodiments 2), 4), and 10).

17) Particular compounds according to embodiment 1) are selected from:

(R)-4-(6-(2-fluoro-4-methoxyphenyl)-2-oxobenzo[d]oxazol-3(2H)-yl)-N-hydroxy-2-methyl-2-(methylsulfonyl)butanamide; and

(R)—N-hydroxy-4-(6-((3-hydroxyoxetan-3-yl)buta-1,3-diyn-1-yl)-2-oxobenzo[d]oxazol-3(2H)-yl)-2-methyl-2-(methylsulfonyl)butanamide.

18) Further particular compounds according to embodiment 1) are selected from:

(R)—N-hydroxy-4-(6-((4-(hydroxymethyl)phenyl)ethynyl)-2-oxobenzo[d]oxazol-3(2H)-yl)-2-methyl-2-(methylsulfonyl)butanamide;

(R)—N-hydroxy-4-(6-((1-(hydroxymethyl)cyclopropyl)buta-1,3-diyn-1-yl)-2-oxobenzo[d]oxazol-3(2H)-yl)-2-methyl-2-(methylsulfonyl)butanamide;

(R)—N-hydroxy-2-methyl-2-(methylsulfonyl)-4-(6-((4-(morpholinomethyl)phenyl)ethynyl)-2-oxobenzo[d]oxazol-3(2H)-yl)butanamide;

(R)—N-hydroxy-2-methyl-4-(6-((1-(methylamino)cyclopropyl)buta-1,3-diyn-1-yl)-2-oxobenzo[d]oxazol-3(2H)-yl)-2-(methylsulfonyl)butanamide;

(R)—N-hydroxy-4-(6-(((1S,2S)-2-(hydroxymethyl)cyclopropyl)buta-1,3-diyn-1-yl)-2-oxobenzo[d]oxazol-3(2H)-yl)-2-methyl-2-(methylsulfonyl)butanamide;

(R)-4-(6-(((1R,2R)-2-fluoro-2-(hydroxymethyl)cyclopropyl)buta-1,3-diyn-1-yl)-2-oxobenzo[d]oxazol-3(2H)-yl)-N-hydroxy-2-methyl-2-(methylsulfonyl)butanamide;

(R)-5-(3-(4-(hydroxyamino)-3-methyl-3-(methylsulfonyl)-4-oxobutyl)-2-oxo-2,3-dihydrobenzo[d]oxazol-6-yl)penta-2,4-diyn-1-yl 3-hydroxyazetidine-1-carboxylate;

(R)-4-(6-((1-aminocyclopropyl)buta-1,3-diyn-1-yl)-2-oxobenzo[d]oxazol-3(2H)-yl)-N-hydroxy-2-methyl-2-(methylsulfonyl)butanamide;

(R)—N-hydroxy-2-methyl-4-(6-((1-methylazetidin-3-yl)buta-1,3-diyn-1-yl)-2-oxobenzo[d]oxazol-3(2H)-yl)-2-(methylsulfonyl)butanamide;

(R)—N-hydroxy-2-methyl-4-(6-((1-methylazetidin-3-yl)buta-1,3-diyn-1-yl)-2-oxobenzo[d]thiazol-3(2H)-yl)-2-(methylsulfonyl)butanamide;

(R)-4-(6-(((1R,2R)-2-fluoro-2-(hydroxymethyl)cyclopropyl)buta-1,3-diyn-1-yl)-2-oxobenzo[d]thiazol-3(2H)-yl)-N-hydroxy-2-methyl-2-(methylsulfonyl)butanamide;

((1S,2S)-2-((3-((R)-4-(hydroxyamino)-3-methyl-3-(methylsulfonyl)-4-oxobutyl)-2-oxo-2,3-dihydrobenzo[d]oxazol-6-yl)buta-1,3-diyn-1-yl)cyclopropyl)methyl dihydrogen phosphate; and

(R)—N-hydroxy-4-(6-((4-(hydroxymethyl)phenyl)ethynyl)-2-oxobenzo[d]thiazol-3(2H)-yl)-2-methyl-2-(methylsulfonyl)butanamide.

The compounds of formula I according to one of embodiments 1) to 18), or pharmaceutically acceptable salts thereof, can be used as medicaments, e.g. in the form of pharmaceutical compositions for enteral (such especially oral) or parenteral administration (including topical application or inhalation).

The production of the pharmaceutical compositions can be effected in a manner which will be familiar to any person skilled in the art (see for example Remington, The Science and Practice of Pharmacy, 21st Edition (2005), Part 5, “Pharmaceutical Manufacturing” [published by Lippincott Williams & Wilkins]) by bringing the described compounds of formula I or their pharmaceutically acceptable salts, optionally in combination with other therapeutically valuable substances, into a galenical administration form together with suitable, non-toxic, inert, therapeutically compatible solid or liquid carrier materials and, if desired, usual pharmaceutical adjuvants.

The compounds of formula I according to one of embodiments 1) to 18), or the pharmaceutically acceptable salts thereof, may therefore be used for the preparation of a medicament, and are suitable, for the prevention or treatment of a bacterial infection, in particular for the prevention or treatment of a bacterial infection caused by Gram-negative bacteria, especially by multi-drug resistant Gram-negative bacteria.

A pharmaceutical composition according to the present invention contains at least one compound of formula I (or a pharmaceutically acceptable salt thereof) as the active ingredient and optionally carriers and/or diluents and/or adjuvants, and may also contain additional known antibiotics.

The compounds of formula I according to one of embodiments 1) to 18), or the pharmaceutically acceptable salts thereof, exhibit antibacterial activity, in a biological environment (i.e. in the presence of a phosphatase, an esterase, a sulfatase or any suitable equivalent thereof capable of removing the group R¹ that is not hydrogen, and capable of removing the phosphono group or other prodrug group such as (di(C₁-C₄)alkylamino)-(C₁-C₃)alkyl-carbonyloxymethyl (e.g. dimethylaminoacetyl) where present in the group M/R^{3A}/R^{1B}), especially against Gram-negative organisms and are therefore suitable to treat bacterial infections in mammals, especially humans. Said compounds may also be used for veterinary applications, such as treating infections in livestock and companion animals. They may further constitute substances for preserving inorganic and organic materials in particular all types of organic materials for example polymers, lubricants, paints, fibres, leather, paper and wood.

The compounds of formula I according to one of embodiments 1) to 18), or the pharmaceutically acceptable salts thereof, may therefore be used for the treatment or prevention of infectious disorders caused by fermentative or non-fermentative Gram negative bacteria, especially those caused by susceptible and multi-drug resistant Gram-negative bacteria. Examples of such Gram-negative bacteria include Acinetobacter spp. such as Acinetobacter baumannii or Acinetobacter haemolyticus, Actinobacillus actinomycetemcomitans, Achromobacter spp. such as Achromobacter xylosoxidans or Achromobacter faecalis, Aeromonas spp. such as Aeromonas hydrophila, Bacteroides spp. such as Bacteroides fragilis, Bacteroides theataioatamicron, Bacteroides distasonis, Bacteroides ovatus or Bacteroides vulgatus, Bartonella hensenae, Bordetella spp. such as Bordetella pertussis, Borrelia spp. such as Borrelia Burgdorferi, Brucella spp. such as Brucella melitensis, Burkholderia spp. such as Burkholderia cepacia, Burkholderia pseudomallei or Burkholderia mallet, Campylobacter spp. such as Campylobacter jejuni, Campylobacter fetus or Campylobacter coli, Cedecea, Chlamydia spp. such as Chlamydia pneumoniae, Chlamydia trachomatis, Citrobacter spp. such as Citrobacter diversus (koseri) or Citrobacter freundii, Coxiella burnetii, Edwardsiella spp. such as Edwarsiella tarda, Ehrlichia chafeensis, Eikenella corrodens, Enterobacter spp. such as Enterobacter cloacae, Enterobacter aerogenes, Enterobacter agglomerans, Escherichia coli, Francisella tularensis, Fusobacterium spp., Haemophilus spp. such as Haemophilus influenzae (beta-lactamase positive and negative) or Haemophilus ducreyi, Helicobacter pylori, Kingella kingae, Klebsiella spp. such as Klebsiella oxytoca, Klebsiella pneumoniae (including those encoding extended-spectrum beta-lactamases (hereinafter “ESBLs”), carbapenemases (KPCs), cefotaximase-Munich (CTX-M), metallo-beta-lactamases, and AmpC-type beta-lactamases that confer resistance to currently available cephalosporins, cephamycins, carbapenems, beta-lactams, and beta-lactam/beta-lactamase inhibitor combinations), Klebsiella rhinoscleromatis or Klebsiella ozaenae, Legionella pneumophila, Mannheimia haemolyticus, Moraxella catarrhalis (beta-lactamase positive and negative), Morganella morganii, Neisseria spp. such as Neisseria gonorrhoeae or Neisseria meningitidis, Pasteurella spp. such as Pasteurella multocida, Plesiomonas shigelloides, Porphyromonas spp. such as Porphyromonas asaccharolytica, Prevotella spp. such as Prevotella corporis, Prevotella intermedia or Prevotella endodontalis, Proteus spp. such as Proteus mirabilis, Proteus vulgaris, Proteus penneri or Proteus myxofaciens, Porphyromonas asaccharolytica, Plesiomonas shigelloides, Providencia spp. such as Providencia stuartii, Providencia rettgeri or Providencia alcalifaciens, Pseudomonas spp. such as Pseudomonas aeruginosa (including ceftazidime-, cefpirome- and cefepime-resistant P. aeruginosa, carbapenem-resistant P. aeruginosa or quinolone-resistant P. aeruginosa) or Pseudomonas fluorescens, Ricketsia prowazekii, Salmonella spp. such as Salmonella typhi or Salmonella paratyphi, Serratia marcescens, Shigella spp. such as Shigella flexneri, Shigella boydii, Shigella sonnei or Shigella dysenteriae, Streptobacillus moniliformis, Stenotrophomonas maltophilia, Treponema spp., Vibrio spp. such as Vibrio cholerae, Vibrio parahaemolyticus, Vibrio vulnificus, Vibrio alginolyticus, Yersinia spp. such as Yersinia enterocolitica, Yersinia pestis or Yersinia pseudotuberculosis.

The compounds of formula I according to one of embodiments 1) to 18), or the pharmaceutically acceptable salts thereof, may thus especially be used for the preparation of a medicament, and are suitable, for the prevention or treatment of a bacterial infection caused by Gram-negative bacteria selected from the group consisting of Acinetobacter baumannii, Burkholderia spp. (e.g. Burkholderia cepacia), Citrobacter spp., Enterobacter aerogenes, Enterobacter cloacae, Escherichia coli, Klebsiella oxytoca, Klebsiella pneumoniae, Serratia marcescens, Stenotrophomonas maltophilia and Pseudomonas aeruginosa (notably for the prevention or treatment of a bacterial infection caused by Acinetobacter baumannii bacteria, Escherichia coli bacteria, Klebsiella pneumoniae bacteria or Pseudomonas aeruginosa bacteria, and in particular for the prevention or treatment of a bacterial infection mediated by quinolone-resistant Acinetobacter baumannii bacteria or quinolone-resistant Klebsiella pneumoniae bacteria).

The compounds of formula I according to one of embodiments 1) to 18), or the pharmaceutically acceptable salts thereof, may more especially be used for the preparation of a medicament, and are suitable, for the prevention or treatment of a bacterial infection caused by Gram-negative bacteria selected from the group consisting of Citrobacter spp., Enterobacter aerogenes, Enterobacter cloacae, Escherichia coli, Klebsiella oxytoca, Klebsiella pneumoniae, Serratia marcescens, Stenotrophomonas maltophilia and Pseudomonas aeruginosa bacteria (notably of a bacterial infection caused by Gram-negative bacteria selected from the group consisting of Klebsiella pneumoniae and Pseudomonas aeruginosa bacteria, and in particular of a bacterial infection caused by Pseudomonas aeruginosa bacteria).

The compounds of formula I according to one of embodiments 1) to 18) are thus useful for treating a variety of infections caused by fermentative or non-fermentative Gram-negative bacteria, especially infections such as: nosocomial pneumonia (related to infection by Legionella pneumophila, Haemophilus influenzae, or Chlamydia pneumoniae); urinary tract infections; systemic infections (bacteraemia and sepsis); skin and soft tissue infections (including burn patients); surgical infections; intraabdominal infections; lung infections (including those in patients with cystic fibrosis); Helicobacter pylori (and relief of associated gastric complications such as peptic ulcer disease, gastric carcinogenesis, etc.); endocarditis; diabetic foot infections; osteomyelitis; otitis media, sinusitus, bronchitis, tonsillitis, and mastoiditis related to infection by Haemophilus influenzae or Moraxella catarrhalis; pharynigitis, rheumatic fever, and glomerulonephritis related to infection by Actinobacillus haemolyticum; sexually transmitted diseases related to infection by Chlamydia trachormatis, Haemophilus ducreyi, Treponema Ureaplasma urealyticum, or Neisseria gonorrheae; systemic febrile syndromes related to infection by Borrelia recurrentis; Lyme disease related to infection by Borrelia burgdorferi; conjunctivitis, keratitis, and dacrocystitis related to infection by Chlamydia trachomatis, Neisseria gonorrhoeae or H. influenzae; gastroenteritis related to infection by Campylobacter jejuni; persistent cough related to infection by Bordetella pertussis and gas gangrene related to infection by Bacteroides spp. Other bacterial infections and disorders related to such infections that may be treated or prevented in accord with the method of the present invention are referred to in J. P. Sanford et al., “The Sanford guide to Antimicrobial Therapy”, 26th Edition, (Antimicrobial Therapy, Inc., 1996).

The compounds of formula I according to one of embodiments 1) to 18), or the pharmaceutically acceptable salts thereof, may thus especially be used for the preparation of a medicament, and are suitable, for the prevention or treatment of a bacterial infection selected from urinary tract infections, systemic infections (such as bacteraemia and sepsis), skin and soft tissue infections (including burn patients), surgical infections; intraabdominal infections and lung infections (including those in patients with cystic fibrosis).

The compounds of formula I according to one of embodiments 1) to 18), or the pharmaceutically acceptable salts thereof, may more especially be used for the preparation of a medicament, and are suitable, for the prevention or treatment of a bacterial infection selected from urinary tract infections, intraabdominal infections and lung infections (including those in patients with cystic fibrosis), and in particular for the prevention or treatment of a bacterial infection selected from urinary tract infections and intraabdominal infections.

The preceding lists of infections and pathogens are to be interpreted merely as examples and in no way as limiting.

Besides, the compounds of formula I according to one of embodiments 1) to 18) may display intrinsic antibacterial properties and have the ability to improve permeability of the outer membrane of Gram-negative bacteria to other antibacterial agents. Their use in combination with another antibacterial agent might offer some further advantages such as lowered side-effects of drugs due to lower doses used or shorter time of treatment, more rapid cure of infection shortening hospital stays, increasing spectrum of pathogens controlled, and decreasing incidence of development of resistance to antibiotics. The antibacterial agent for use in combination with a compound of formula I according to one of embodiments 1) to 18) will be selected from the group consisting of a penicillin antibiotic (such as ampicillin, piperacillin, penicillin g, amoxicillin, or ticarcillin), a cephalosporin antibiotic (such as ceftriaxone, cefatazidime, cefepime, cefotaxime) a carbapenem antibiotic (such as imipenem, or meropenem), a monobactam antibiotic (such as aztreonam), a fluoroquinolone antibiotic (such as ciprofloxacin, moxifloxacin or levofloxacin), a macrolide antibiotic (such as erythromycin or azithromycin), an aminoglycoside antibiotic (such as amikacin, gentamycin or tobramycin), a glycopeptide antibiotic (such as vancomycin or teicoplanin), a tetracycline antibiotic (such as tetracycline, oxytetracycline, doxycycline, minocycline or tigecycline), and linezolid, clindamycin, telavancin, daptomycin, novobiocin, rifampicin and polymyxin. Preferably, the antibacterial agent for use in combination with a compound of formula I according to one of embodiments 1) to 18) will be selected from the group consisting of vancomycin, tigecycline and rifampicin.

The compounds of formula I according to one of embodiments 1) to 18), or the pharmaceutically acceptable salt thereof, may moreover be used for the preparation of a medicament, and are suitable, for the prevention or treatment (and especially the treatment) of infections caused by biothreat Gram negative bacterial pathogens as listed by the US Center for Disease Control (the list of such biothreat bacterial pathogens can be found at the web page http://ww.selectagents.gov/SelectAgentsandToxinsList.html), and in particular by Gram negative pathogens selected from the group consisting of Yersinia pestis, Francisella tularensis (tularemia), Burkholderia pseudomallei and Burkholderia mallei.

As well as in humans, bacterial infections can also be treated using compounds of formula I according to one of embodiments 1) to 18) (or pharmaceutically acceptable salts thereof) in other species like pigs, ruminants, horses, dogs, cats and poultry.

Another aspect of the invention concerns a method for the prevention or the treatment of a Gram-negative bacterial infection in a patient, comprising the administration to said patient of a pharmaceutically active amount of a compound of formula I according to one of embodiments 1) to 18), or a pharmaceutically acceptable salt thereof. Accordingly, the invention provides a method for the prevention or the treatment of a bacterial infection caused by Gram-negative bacteria (notably for the prevention or treatment of a bacterial infection caused by Acinetobacter baumannii bacteria, Escherichia coli bacteria, Klebsiella pneumoniae bacteria or Pseudomonas aeruginosa bacteria, and in particular for the prevention or treatment of a bacterial infection caused by quinolone-resistant Acinetobacter baumannii quinolone-resistant bacteria or Klebsiella pneumoniae quinolone-resistant bacteria) in a patient, comprising the administration to said patient of a pharmaceutically active amount of a compound of formula I according to one of embodiments 1) to 18), or a pharmaceutically acceptable salt thereof.

Moreover, the compounds of formula I according to one of embodiments 1) to 18) may also be used for cleaning purposes, e.g. to remove pathogenic microbes and bacteria from surgical instruments, catheters and artificial implants or to make a room or an area aseptic. For such purposes, the compounds of formula I could be contained in a solution or in a spray formulation.

It is understood that compounds of formula I wherein R¹ is not H or wherein the group M comprises a phosphonooxy group or another prodrug group such as (di(C₁-C₄)alkylamino)-(C₁-C₃)alkyl-carbonyloxy (e.g. dimethylaminoacetoxy), [(2-(phosphonooxy-(C₁-C₃)alkyl)-phenyl)-(C₁-C₃)alkyl]-carbonyloxy, [2-(phosphonooxy-(C₁-C₃)alkyl)-phenyl]-carbonyloxy, or [(2-phosphonooxy-phenyl)-(C₁-C₃)alkyl]-carbonyloxy (e.g. [2-(2-phosphonooxy-phenyl)-ethyl]-carbonyloxy) may require bioactivation by phosphatases and/or esterases and/or any biological system to exert their antibacterial activity upon administration to humans.

Besides, the term “room temperature” as used herein refers to a temperature of 25° C.

Unless used regarding temperatures, the term “about” placed before a numerical value “X” refers in the current application to an interval extending from X minus 10% of X to X plus 10% of X, and preferably to an interval extending from X minus 5% of X to X plus 5% of X. In the particular case of temperatures, the term “about” placed before a temperature “Y” refers in the current application to an interval extending from the temperature Y minus 10° C. to Y plus 10° C., and preferably to an interval extending from Y minus 5° C. to Y plus 5° C.

The compounds of formula I can be manufactured in accordance with the present invention using the procedures described hereafter.

### Preparation of the Compounds of Formula I

### Abbreviations:

The following abbreviations are used throughout the specification and the examples:
Ac acetyl
AcOH acetic acid
aq. aqueous
CC column chromatography over silica gel
Cipro ciprofloxacin
conc. concentrated
Cy cyclohexyl
DAD diode array detection
dba dibenzylideneacetone
DCC dicyclohexylcarbodiimide
DCM dichloromethane
DME 1,2-dimethoxyethane
DMF N,N-dimethylformamide
DMSO dimethylsulfoxide
dppf 1,1′-bis(diphenylphosphino)ferrocene
EA ethyl acetate
EDC N-(3-dimethylaminopropyl)-N′-ethylcarbodiimide hydrochloride
ELSD evaporative light scattering detector
ESI electron spray ionisation
eq. equivalent
Et ethyl
Et₂O diethyl ether
EtOH ethanol
HATU O-(7-azabenzotriazol-1-yl)-N,N,N′,N′-tetramethyluronium hexafluorophosphate
Hept heptane
Hex hexane
HOBT hydroxybenzotriazole
HPLC high pressure liquid chromatography
iPr iso-propyl
IT internal temperature
LC liquid chromatography
MCPBA meta-chloroperbenzoic acid
Me methyl
MeCN acetonitrile
MeOH methanol
MS mass spectroscopy
NBS N-bromosuccinimide
NMR Nuclear Magnetic Resonance
org. organic
Pd/C palladium on carbon
PEPPSI™-IPr [1,3-bis(2,6-diisopropylphenyl)imidazol-2-ylidene](3-chloropyridyl)palladium(II) dichloride
PG protecting group
Ph phenyl
PPTS para-toluenesulfonic acid pyridinium salt
prep-HPLC preparative HPLC
Pyr pyridine
quant. quantitative
Q-phos 1,2,3,4,5-pentaphenyl-1′-(di-tert-butylphosphino)ferrocene
RE Reference Example
rt room temperature
sat. saturated
SK-CC01-A 2′-(dimethylamino)-2-biphenylyl-palladium(II) chloride dinorbornylphosphine complex
S-Phos 2-dicyclohexylphosphino-2′,6′-dimethoxybiphenyl
TBAF tetra-n-butylammonium fluoride
TBDPS tert-butyldiphenylsilyl
TBME tert-butylmethyl ether
tBu tert-butyl
TEA triethylamine
TFA trifluoroacetic acid
THF tetrahydrofuran
THP tetrahydropyranyl
TLC thin layer chromatography
TMS trimethylsilyl
TMSE 2-(trimethylsilyl)ethyl
t_{R} retention time
trityl triphenylmethyl

### General Reaction Techniques:

### General Reaction Technique 1 (Hydroxamic Acid Protecting Group Removal):

The protecting groups of hydroxamic acid ester derivatives (—CO—NH—OPG), the protecting groups of phosphonic acid ester derivatives (P(O)(OPG′)₂ are removed as follows:
When PG or PG′ is THP, (2-methylpropoxy)ethyl, methoxymethyl, tBu, COOtBu or COtBu: by acidic treatment with e.g. TFA or HCl in an org. solvent such as DCM, dioxane, Et₂O or MeOH between 0° C. and rt or by treatment with pyridinium para-toluenesulfonate in EtOH between rt and +80° C.;
When PG or PG is trityl: by treatment with diluted acid such as citric acid or HCl in an org. solvent such as MeOH or DCM;
When PG or PG′ is TMSE: by using fluoride anion sources such as BF₃.etherate complex in MeCN at 0° C., TBAF in THF between 0° C. and +40° C. or HF in MeCN or water between 0° C. and +40° C., or using acidic conditions such as AcOH in THF/MeOH or HCl in MeOH;
When PG or PG′ is allyl: by treatment with Pd(PPh₃)₄ in a solvent such as MeOH in presence of K₂CO₃ or a scavenger such as dimedone, morpholine or tributyltin hydride;

Further general methods to remove hydroxamic acid protecting groups have been described in T. W. Greene & P. G. M. Wuts, Protecting Groups in Organic Synthesis, 3rd Ed (1999), 23-147 (Publisher: John Wiley and Sons, Inc., New York, N.Y.).

### General Reaction Technique 2 (Amide Bond Formation):

The carboxylic acid is reacted with the hydroxylamine derivative in the presence of an activating agent such as DCC, EDC, HOBT, n-propylphosphonic cyclic anhydride, HATU or di-(N-succinimidyl)-carbonate, in a dry aprotic solvent such as DCM, MeCN or DMF between −20° C. and 60° C. (see g. Benz in Comprehensive Organic Synthesis, B. M. Trost, I. Fleming, Eds; Pergamon Press: New York (1991), vol. 6, p. 381). Alternatively, the carboxylic acid can be activated by conversion into its corresponding acid chloride by reaction with oxalyl chloride or thionyl chloride neat or in a solvent like DCM between −20° and 60° C. Further activating agents can be found in R. C. Larock, Comprehensive Organic Transformations. A guide to Functional group Preparations, 2nd Edition (1999), section nitriles, carboxylic acids and derivatives, p. 1941-1949 (Wiley-VC; New York, Chichester, Weinheim, Brisbane, Singapore, Toronto).

### General Reaction Technique 3 (Suzuki Cross-Coupling):

The aromatic halide (typically a bromide) is reacted with the required boronic acid derivative or its boronate ester equivalent (e.g. pinacol ester) in the presence of a palladium catalyst and a base such as K₂CO₃, Cs₂CO₃, K₃PO₄, tBuONa or tBuOK between 20 and 120° C. in a solvent such as toluene, THF, dioxane, DME or DMF, usually in the presence of water (20 to 50%). Examples of typical palladium catalysts are triarylphosphine palladium complexes such as Pd(PPh₃)₄. These catalysts can also be prepared in situ from a common palladium source such as Pd(OAc)₂ or Pd₂(dba)₃ and a ligand such as trialkylphosphines (e.g. PCy₃ or P(tBu)₃), dialkylphosphinobiphenyls (e.g. S-Phos) or ferrocenylphosphines (e.g. Q-phos). Alternatively, one can use a commercially available precatalyst based on palladacycle (e.g. SK-CC01-A) or N-heterocyclic carbene complexes (e.g. PEPPSI™-IPr). The reaction can also be performed by using the corresponding aromatic triflate. Further variations of the reaction are described in Miyaura and Suzuki, Chem. Rev. (1995), 95, 2457-2483, Bellina et al., Synthesis (2004), 2419-2440, Mauger and Mignani, Aldrichimica Acta (2006), 39, 17-24, Kantchev et al., Aldrichimica Acta (2006), 39, 97-111, Fu, Acc. Chem. Res. (2008), 41, 1555-1564, and references cited therein.

General Reaction Technique 4 (alkyne-alkyne Cross-Coupling, haloaryl-alkyne or alkyne-haloalkyne Cross-Coupling):

An alkyne derivative is coupled with a second alkyne or a haloalkyne derivative, using a catalytic amount of a palladium salt, an org. base such as TEA and a catalytic amount of a copper derivative (usually copper iodide) in a solvent such as DMF at a temperature from 20 to 100° C. (see Sonogashira, K. in Metal-Catalyzed Reactions, Diederich, F., Stang, P. J., Eds.; Wiley-VCH: New York (1998)). Alternatively, the alkyne-haloalkyne cross coupling reaction can be performed using only a catalytic amount of copper derivative in presence of aqueous hydroxylamine and a base such as piperidine or pyrrolidine (see Chodkiewicz and Cadiot, C. R. Hebd. Seances Acad. Sci. (1955), 241, 1055-1057).

General Reaction Technique 5 (Transformation of an Ester into an Acid):

When the ester side chain is a linear alkyl, the hydrolysis is usually performed by treatment with an alkali hydroxide such as LiOH, KOH or NaOH in a water-dioxan or water THF mixture between 0° C. and 80° C. When the ester side chain is tBu, the release of the corresponding acid can also be performed in neat TFA or diluted TFA or HCl in an org. solvent such as ether or THF. When the ester side chain is the allyl group, the reaction is performed in the presence of tetrakis(triphenylphosphine)palladium(0) in the presence of an allyl cation scavenger such as morpholine, dimedone or tributyltin hydride between 0° C. and 50° C. in a solvent such as THF. When the ester side chain is benzyl, the reaction is performed under hydrogen in the presence of a noble metal catalyst such as Pd/C in a solvent such as MeOH, THF or EA. Further strategies to introduce other acid protecting groups and general methods to remove them have been described in T. W. greene & P. G. M. Wuts, Protecting groups in Organic Synthesis, 3^{rd} Ed. (1999), 369-441 (Publisher: John Wiley and Sons, Inc., New York, N.Y.).

### General Preparation Methods:

### Preparation of the Compounds of Formula I:

The compounds of formula I can be manufactured by the methods given below, by the methods given in the examples or by analogous methods. Optimum reaction conditions may vary with the particular reactants or solvents used, but such conditions can be determined by a person skilled in the art by routine optimisation procedures.

The sections hereafter describe general methods for preparing compounds of formula I. If not indicated otherwise, the generic groups V, R¹, R², R³, R⁴, L, M, M^{A}, M^{B}, R^{1A}, R^{2a}, R^{3A} and R^{1B} are as defined for formula I. General synthetic methods used repeatedly throughout the text below are referenced to and described in the above section entitled “General reaction techniques”. In some instances certain generic groups might be incompatible with the assembly illustrated in the procedures and schemes below and so will require the use of protecting groups. The use of protecting groups is well known in the art (see for example T. W. greene, P. G. M. Wuts, Protective Groups in Organic Synthesis, 3^{rd} Ed (1999), Wiley-Interscience).

The compounds of formula I wherein R¹═H can be obtained by deprotecting a compound of formula II

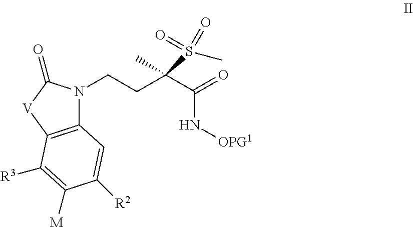

wherein PG¹ represents THP, TMSE, benzyl, trityl, (2-methylpropoxy)ethyl, methoxymethyl, allyl, tBu, COOtBu or COtBu using general reaction technique 1. The reaction can also be performed with racemic material and the (R) enantiomer can be obtained by chiral HPLC separation.

### Preparation of the Compounds of Formula I Wherein R¹≠H.

The compounds of formula I wherein R¹≠H can be obtained by:

a) reacting a compound of formula I wherein R¹═H with a compound of formula III

(PG^{A}O)₂P—N(iPr)₂ III

wherein PGA represents tert-butyl or benzyl. The reaction is performed in presence of a base such as tetrazole in a solvent such as acetonitrile at a temperature in the vicinity of 0° C. An oxidation reaction is subsequently performed using hydrogen peroxide in water or MCPBA as an oxidizing agent. This sequence can also be performed with racemic compound of formula I and the (R)-enantiomer can then be obtained by chiral HPLC separation of the reaction product. Functional groups (e.g. amino or hydroxy) present on R³ that would be incompatible with the reaction conditions abovementioned can be protected (as carbamates or THP/silyl/tert-butyl ethers respectively) before performing said reaction and deprotected after performing said reaction. Final cleavage of PG^{A} can be performed using general reaction technique 5, leading to compounds of formula I wherein R¹═PO₃H₂.

b) reacting a compound of formula I wherein R¹═H with a compound of formula IV.

HOC(O)R⁴ IV

The reaction can be performed using general reaction technique 2 leading to compounds of Formula I wherein R¹═C(O)R⁴. This reaction can also be performed with racemic compound of formula I and the (R)-enantiomer can then be obtained by chiral HPLC separation of the reaction product. Functional groups (e.g. amino or hydroxy) present on R⁴ and M that would be incompatible with the reaction conditions abovementioned can be protected (as carbamates or THP/silyl/tert-butyl ethers respectively) before performing said reaction and deprotected after performing said reaction.

c) reacting a compound of formula I wherein R¹═H with a compound of formula V

X^{a}—(CH₂)—O—P(O)(OPG^{A})₂ V

wherein X^{a} represents iodine, bromine or chlorine and PGA has the same meaning as in formula III. The reaction can be performed in presence of a mineral base such as NaH or K₂CO₃ or in presence of an organic base such as TEA or DIPEA in a solvent such as THF at a temperature ranging between about −50° C. and rt. Functional groups (e.g. amino or hydroxy) present on M that would be incompatible with the reaction conditions abovementioned can be protected (as carbamates or THP/silyl/tert-butyl ethers respectively) before performing said reaction and deprotected after performing said reaction. This sequence can also be performed with racemic compound of formula I and the (R)-enantiomer can then be obtained by chiral HPLC separation of the reaction product. Final cleavage of PG^{A} can be performed using general reaction technique 5 leading to compounds of formula I wherein R¹═CH₂—O—PO₃H₂.

d) reacting a compound of formula I wherein R¹═H with Pyr.SO₃ complex or Me₂NCHO.SO₃ complex in a solvent such as DMF or pyridine leading to compounds of formula I wherein R¹═SO₃H. Functional groups (e.g. amino or hydroxy) present on M that would be incompatible with the reaction conditions abovementioned can be protected (as carbamates or THP/silyl/tert-butyl ethers respectively) before performing said reaction and deprotected after performing said reaction. This sequence can also be performed with racemic compound of formula I and the (R)-enantiomer can then be obtained by chiral HPLC separation of the reaction product.

If desired, the compounds of formula I thus obtained may be converted into their salts, and notably into their pharmaceutically acceptable salts using standard methods.

Besides, whenever the compounds of formula I are obtained in the form of mixtures of enantiomers, the enantiomers can be separated using methods known to one skilled in the art, e.g. by formation and separation of diastereomeric salts or by HPLC over a chiral stationary phase such as a Regis Whelk-O1(R,R) (10 μm) column, a Daicel ChiralCel OD-H (5-10 μm) column, or a Daicel ChiralPak IA (10 μm) or AD-H (5 μm) column. Typical conditions of chiral HPLC are an isocratic mixture of eluent A (EtOH, in the presence or absence of an amine such as TEA or diethylamine) and eluent B (Hex), at a flow rate of 0.8 to 150 mL/min. Particular conditions are set out in the experimental part below.

### Preparation of the Compounds of Formula II:

The compounds of formula II can be obtained by:

a) reacting a compound of formula VI with a compound of formula VII

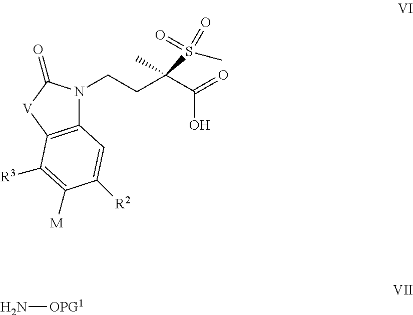

wherein PG¹ has the same meaning as in formula II using general reaction technique 2 (this reaction can also be performed with racemic compound of formula VI and the (R)-enantiomer can then be obtained by chiral HPLC separation of the reaction product), whereby functional groups (e.g. amino or hydroxy) present on M that would be incompatible with the coupling conditions mentioned in general reaction technique 2 can be protected (as carbamates or THP/silyl ethers respectively) before performing said reaction and deprotected after performing said reaction; or

b) reacting a boron derivative of formula VIII

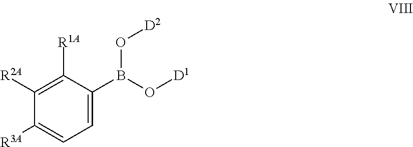

wherein D¹ and D² represent H, methyl or ethyl or D¹ and D² together represent CH₂C(Me)₂CH₂ or C(Me)₂C(Me)₂ with a compound of formula IX

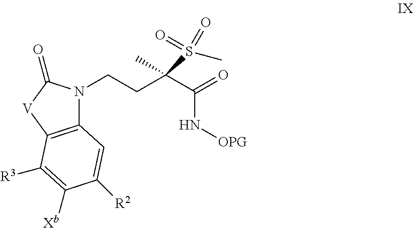

wherein X^{b} represents a halogen such as bromine or iodine and PG has the same meaning as in formula II, using general reaction technique 3 (this reaction can also be performed with racemic compound of formula IX and the (R)-enantiomer can then be obtained by chiral HPLC separation of the reaction product); or

c) reacting a compound of formula X

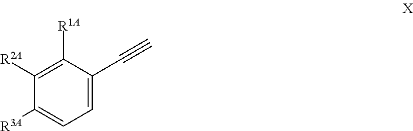

with a compound of formula VIII as defined in section b) above wherein X^{b} represents iodine, using general reaction technique 4 (this reaction can also be performed with racemic compound of formula IX and the (R)-enantiomer can then be obtained by chiral HPLC separation of the reaction product); or

d) reacting a compound of formula XI

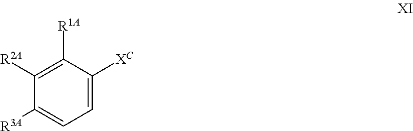

wherein X^{c} represents iodine or bromine (and preferably iodine), with a compound of formula VIIIa

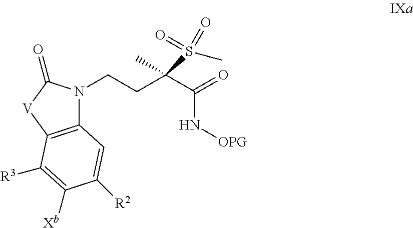

wherein X^{b} represents ethynyl and PG has the same meaning as in formula II, using general reaction technique 4 (this reaction can also be performed with racemic compound of formula IXa and the (R)-enantiomer can then be obtained by chiral HPLC separation of the reaction product); or

e) reacting a compound of formula XII

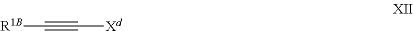

wherein X^{d} represents iodine or bromine, with a compound of formula IXa as defined in section d) above, using general reaction technique 4 (this reaction can also be performed with racemic compound of formula IXa and the (R)-enantiomer can then be obtained by chiral HPLC separation of the reaction product).

### Preparation of the Synthesis Intermediates of Formulae VI, VII, VIII, IX, IXa, X, XI and XII:

### Compounds of Formula VI:

The compounds of formula VI can be prepared as summarised in Scheme 1 hereafter.

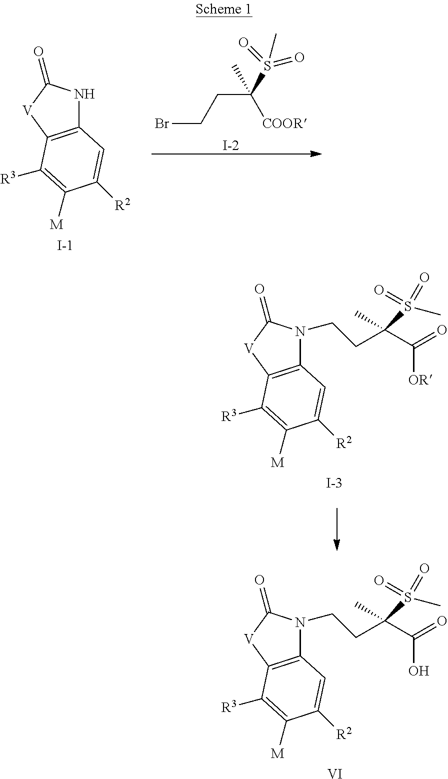

In Scheme 1, R′ is Et or tBu. The reactions can also be performed with racemic material and the (R)-enantiomer can be obtained by chiral HPLC separation at any step when suitable.

The derivatives of formula I-3 can be obtained (Scheme 1) by reaction of derivatives of formula I-1 with the bromides of formula I-2 in presence of a base such as K₂CO₃ in a solvent such as DMF at a temperature ranging between rt and 80° C. and preferably at about 80° C. The derivatives of formula I-3 can be transformed to the compounds of formula VI using general reaction technique 5.

### Compounds of Formula VII:

The compounds of formula VII are commercially available (PG¹=TMSE, trityl, methoxymethyl, THP, tBu, COOtBu or allyl) or can be prepared according to WO 2010/060785 (PG¹=(2-methylpropoxy)ethyl) or Marmer and Maerker, J. Org. Chem. (1972), 37, 3520-3523 (PG¹=COtBu).

### Compounds of Formula VIII:

The compounds of formula VIII wherein A is a bond and D¹ and D² each represent H or (C₁-C₂)alkyl are commercially available or can be prepared according to Sleveland et al., Organic Process Research & Development (2012), 16, 1121-1130 starting from tri((C₁-C₂)alkyl)borate and the corresponding commercially available bromo derivatives (optionally followed by acidic hydrolysis). The compounds of formula VIII wherein A represents a bond and D¹ and D² together represent CH₂C(Me)₂CH₂ or C(Me)₂C(Me)₂ are commercially available or can be prepared according to WO 2012/093809, starting from bis(pinacolato)diborane or 5,5-dimethyl-1,3,2-dioxaborinane (both commercially available) with the corresponding commercially available bromo derivatives of formula VIII.

### Compounds of Formulae IX and IXa:

The compounds of formulae IX and IXa can be prepared as summarised in Scheme 2 hereafter.

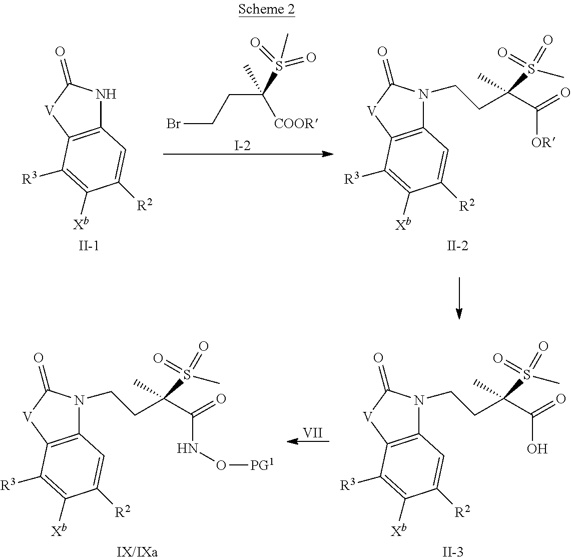

In Scheme 2, R′ is Et or tBu, X^{b} represents halogen (such as iodine or bromine) or ethynyl and PG¹ has the same meaning as in formula II. The reactions can also be performed with racemic material and the (R)-enantiomer can be obtained by chiral HPLC separation at any step when suitable.

The derivatives of formula II-2 can be obtained (Scheme 2) by reaction of derivatives of formula II-1 with the bromides of formula I-2 in presence of a base such as K₂CO₃ in a solvent such as DMF at a temperature ranging between rt and about 80° C. and preferably at about 80° C. The compounds of formulae II-1 and II-2 wherein X^{b}═Br can be transformed to the corresponding compounds of formulae II-1 or II-2 wherein X^{b}=iodine by reaction with NaI in the presence of a copper (I) salt and a ligand such as trans-N,N′-dimethylcyclohexa-1,2-diamine in a solvent such as dioxane at a temperature ranging between rt and 100° C., or in a microwave oven at about 150° C. The compounds of formula II-2 wherein X^{b}=ethynyl can be obtained by reaction of compounds of formula II-2 wherein X^{b}=iodine with trimethylsilylacetylene (III-1) using general reaction technique 4 followed by treatment with TBAF in THF. The derivatives of formula II-3 can be obtained from the derivatives of formula II-2 using general reaction technique 5. The compounds of formula II-3 can be reacted with the compounds of formula VII to afford the compounds of formula IX or IXa using general reaction technique 2. The compounds of formula IXa wherein X^{b}=ethynyl can be obtained by reaction of compounds of formula IX wherein X^{b}=bromine or iodine using the abovementioned sequence.

### Compounds of Formula X.

The compounds of formula X are commercially available or can be prepared as summarised in Scheme 3 hereafter.

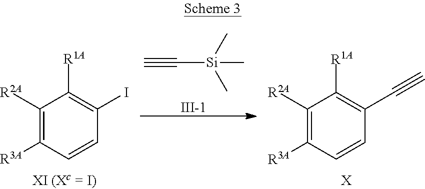

The compounds of formula XI wherein X^{c} represents iodine can be reacted (Scheme 3) with trimethylsilylacetylene (III-1) using general reaction technique 4 followed by treatment with TBAF in THF, affording the derivatives of formula XI.

### Compounds of Formula XI:

The compounds of formula XI wherein X^{c} represents bromine are commercially available or can be prepared by standard methods known to one skilled in the art. The compounds of formula XI wherein X^{c} represents iodine can be obtained from the corresponding bromine derivatives by reaction with NaI in the presence of a copper (I) salt and a ligand such as trans-N,N′-dimethylcyclohexa-1,2-diamine in a solvent such as dioxane at a temperature ranging between rt and 100° C., or in a microwave oven at about 150° C.

### Compounds of Formula XII:

The compounds of formula XII wherein X^{d} represents iodine can be prepared by iodination of the corresponding compounds wherein X^{d} would be H with iodine in the presence of an inorganic base such as KOH. The compounds of formula XII wherein X^{d} represents bromine can be prepared from the corresponding compounds wherein X^{d} would be H by treatment with NBS in the presence of AgNO₃ in a solvent such as acetone or MeCN.

### Other Synthesis Intermediates and Starting Materials:

The compounds of formula II-1 are commercially available or can be prepared by standard methods known to one skilled in the art.

The compound of formula I-2 can be prepared in analogy to the methods described in the experimental section below (see Preparation A and B), or by standard methods known to one skilled in the art.

Particular embodiments of the invention are described in the following Examples, which serve to illustrate the invention in more detail without limiting its scope in any way.

### EXAMPLES

All temperatures are stated in ° C. Unless otherwise indicated, the reactions take place at rt. The combined org. layers resulting from the liquid-liquid extraction during the work-up procedure of a reaction mixture are, unless otherwise indicated, washed with a minimal volume of brine, dried over MgSO₄, filtered and evaporated to dryness to provide a so-called evaporation residue.

Analytical TLC characterisations were performed with 0.2 mm plates: Merck, Silica gel 60 F₂₅₄. Elution is performed with EA, Hept, DCM, MeOH or mixtures thereof. Detection was done with UV or with a solution of KMnO₄ (3 g), K₂CO₃ (20 g), 5% NaOH (3 mL) and H₂O (300 mL) with subsequent heating.

CCs were performed using Brunschwig 60A silica gel (0.032-0.63 mm) or using an ISCO CombiFlash system and prepacked SiO₂ cartridges, elution being carried out with either Hept-EA or DCM-MeOH mixtures with an appropriate gradient. When the compounds contained an acid function, 1% of AcOH was added to the eluent(s). When the compounds contained a basic function, 25% aq. NH₄OH was added to the eluents.

The compounds were characterized by ¹-NMR (300 MHz, Varian Oxford; 400 MHz, Bruker Avance 400 or 500 MHz, Bruker Avance 500 Cryoprobe). Chemical shifts δ are given in ppm relative to the solvent used; multiplicities: s=singlet, d=doublet, t=triplet, q=quartet, p=pentet, hex=hexet, hep=heptet, m=multiplet, br.=broad; coupling constants J are given in Hz. Alternatively compounds were characterized by LC-MS (Sciex API 2000 with Agilent 1100 Binary Pump with DAD and ELSD or an Agilent quadrupole MS 6140 with Agilent 1200 Binary Pump, DAD and ELSD); by TLC (TLC plates from Merck, Silica gel 60 F₂₅₄); or by melting point.

The analytical LC-MS data have been obtained using the following respective conditions:
Column: Zorbax SB-Aq, 30.5 μm, 4.6×50 mm;
Injection volume: 1 μL;
Column oven temperature: 40° C.;
Detection: UV 210 nm, ELSD and MS;
MS ionization mode: ESI+;
Eluents: A: H₂O+0.04% TFA; and B: MeCN;
Flow rate: 40.5 mL/min;
Gradient: 5% B to 95% B (0.0 min-1.0 min), 95% B (1.0 min-1.45 min).

The number of decimals given for the corresponding [M+H⁺] peak(s) of each tested compound depends upon the accuracy of the LC-MS device actually used.

The prep-HPLC purifications were performed on a gilson HPLC system, equipped with a Gilson 215 autosampler, Gilson 333/334 pumps, Dionex MSQ Plus detector system, and a Dionex UVD340U (or Dionex DAD-3000) UV detector, using the following respective conditions:

Method 1:
Column: Waters Atlantis T3 OBD, 10 μm, 30×75 mm;
Flow rate: 75 mL/min;
Eluents: A: H₂O+0.1% HCOOH; B: MeCN+0.1% HCOOH;
Gradient: 90% A to 5% A (0.0 min-4.0 min), 5% A (4.0 min-6.0 min).

Method 2:
Column: Waters XBridge C18, 10 μm, 30×75 mm;
Flow rate: 75 mL/min;
Eluents: A: H₂O+25% aq. NH₄OH solution (0.5% v/v); B: MeCN;
Gradient: 90% A to 5% A (0.0 min-4.0 min), 5% A (4.0 min-6.0 min).

Method 3:
Column: Waters XBridge C18, 10 μm, 30×75 mm;
Flow rate: 75 mL/min;
Eluents: A: H₂O+0.5% HCOOH; B: MeCN;
Gradient: 90% A to 5% A (0.0 min-4.0 min), 5% A (4.0 min-6.0 min).

Semi-preparative chiral HPLCs were performed using the conditions herafter.

### Semi-Preparative Chiral HPLC Method A:

The semi-preparative chiral HPLC is performed on a Daicel ChiralPak AS-H column (250×30 mm, 5 μm) using the eluent mixture, flow rate and detection conditions indicated between brackets in the corresponding experimental protocol. The retention times are obtained by elution of analytical samples on a Daicel ChiralPak AS-H column (250×4.6 mm, 5 μm) using the same eluent mixture with the flow rate indicated between brackets in the corresponding experimental protocol.

### Semi-Preparative Chiral HPLC Method B:

The semi-preparative chiral HPLC is performed on a Daicel ChiralPak AY-H column (20×250 mm, 5 μm) using the eluent mixture, flow rate and detection conditions indicated between brackets in the corresponding experimental protocol. The retention times are obtained by elution of analytical samples on a Daicel ChiralPak AY-H column (4.6×250 mm, 5 μm) using the same eluent mixture with the flow rate indicated between brackets in the corresponding experimental protocol.

### Procedures:

### Procedure A:

A mixture of the bromo derivative (0.44 mmol), the phenylboronic acid or boronate ester derivative (0.44 mmol), K₂CO₃ (0.105 g; 0.75 mmol) and Pd(PPh₃)₄ (0.055 g; 0.047 mmol) is flushed with nitrogen for 15 min. Dioxane (4.8 mL) and water (0.7 mL) are added and the mixture is refluxed for 1 h. After cooling, water (15 mL) and EA (20 mL) are added and the two layers are separated. The aq. layer is extracted with EA (2×20 mL). The evaporation residue is then purified by CC (Hept-EA or DCM-MeOH).

### Procedure B:

To the THP-protected hydroxamic acid derivative (0.41 mmol) in EtOH (3 mL) is added PPTS (0.25 mmol). The mixture is stirred at 80° C. for 2 h, cooled to rt and directly purified by CC (DCM-MeOH) or by prep HPLC using a suitable method or by a suitable prep-HLC method.

### Procedure C:

CuI (0.035 mmol), PdCl₂(PPh₃)₂ (0.02 mmol), the terminal alkyne derivative (0.17 mmol) and the iodoalkyne (0.18 mmol) are introduced in a two-necked round flask. The atmosphere is flushed with nitrogen during 30 min, then degassed THF (1.5 mL) and degassed TEA (0.43 mmol) are added. The suspension is stirred under nitrogen atmosphere at rt° C. for 16 h. After concentration to dryness, the residue is then purified by CC (Hept-EA or DCM-MeOH) or by a suitable prep-HLC method.

### Procedure D:

CuI (0.1 mmol), PdCl₂(PPh₃)₂ (0.04 mmol), the iodo derivative (0.34 mmol) and the terminal alkyne derivative (0.36 mmol) are introduced in a two-necked round flask. The atmosphere is flushed with nitrogen during 30 min, then degassed THF (2 mL) and degassed TEA (1.22 mmol) are added. The suspension is stirred under nitrogen atmosphere at 50° C. for 4 h. After concentration to dryness, the residue is purified either by CC (Hept-EA or DCM-MeOH) or by a suitable prep-HPLC.

### Procedure E:

To the THP-protected hydroxamic acid derivative (0.169 g, 0.28 mmol) in H₂O (0.5 mL) was added TFA (0.8 mL). After one hour stirring at rt, the mixture was directly purified by prep-HPLC using a suitable method.

### Procedure F:

CuCl (0.046 mmol) and NH₂OH.HCl (0.63 mmol) are dissolved in n-BuNH₂ (30% in water, 0.17 mL). The terminal alkyne (0.344 mmol) is added and the reaction mixture is ice-chilled. Halo-alkyne (0.462 mmol) in dioxane (0.2 mL) is added and the reaction proceeds 1 h at 0° C. The reaction mixture is then allowed to warm up to rt over 1 h. Water (5 mL) and EA (30 mL) are added and two phases are separated. The aq. layer is extracted with EA (10 mL). The evaporation residue is then purified by CC or by prep-HPLC using a suitable method to afford the bis-alkyne product.

### Procedure G:

To a solution of the THP-protected hydroxamic acid derivative (0.22 mmol) in MeOH (1.2 mL) and water (0.4 mL) is added 2M HCl (0.7 mL; 1.2 mmol). The reaction mixture is stirred at rt until completion. The reaction mixture, after neutralization with sat. NaHCO₃ sol. is extracted with DCM-MeOH (9-1, 3×20 mL). The evaporation residue is then purified by CC (DCM-MeOH) or by prep-HPLC using a suitable method.

### Procedure H:

A solution of the THP-protected hydroxamic acid derivative (0.22 mmol) in 4M HCl in dioxane (0.3 mL) was stirred 10 min at rt. The mixture was directly purified by prep-HPLC using a suitable method.

### PREPARATIONS

### Preparation A: (2RS)-tert-butyl 4-bromo-2-methyl-2-(methylsulfonyl)butanoate

### A.i. (2RS)-tert-butyl 2-(methylsulfonyl)propanoate

To a suspension of sodium methanesulfinate (100 g; 929 mmol) in tBuOH (350 mL) was added tert-butyl-2-bromopropionate (150 mL; 877 mmol). The reaction mixture was stirred at about 90° C. for 24 h under nitrogen atmosphere, then cooled to rt and concentrated to dryness. The residue was partitioned between water (750 mL) and EA (600 mL). The aq. layer was extracted with EA (2×500 mL). The evaporation residue afford the title compound as a white yellow solid (175 g, 96% yield).

¹H-NMR (d₆-DMSO) δ: 4.24 (q, J=7.2 Hz, 1H); 3.11 (s, 3H); 1.45 (s, 9H); 1.40 (d, J=7.2 Hz, 3H).

### A.ii. (2RS)-tert-butyl 4-bromo-2-methyl-2-(methylsulfonyl)butanoate

To an ice-chilled suspension of intermediate A.i (130 g; 626 mmol) in DMF (750 mL) was added portionwise NaH (60% in mineral oil; 32.1 g; 802 mmol) for 1.5 h, keeping the temperature below 7° C. The mixture was stirred at 0° C. for 1.5 h, allowed to reach rt and stirred at rt for 0.5 h. The mixture was cooled down to 12° C. with an ice bath and 1,2-dibromoethane (166 mL; 1.9 mol) was then added dropwise, keeping the temperature below 22° C. The reaction mixture was stirred at rt for 2 h. The mixture was poured into cold water (1 L) and Et₂O (1 L) and the aq. layer was extracted with Et₂O (2×750 mL). The org. layer was washed with cold water (2×500 mL). The evaporation residue was purified by CC (Hept-EA) to afford the title compound as a pale yellowish oil (116.8 g; 59% yield).

¹NMR (d6-DMSO) δ: 3.63-3.71 (m, 1H); 3.45-3.37 (m, 1H); 3.12 (s, 3H); 2.72-2.62 (m, 1H); 2.43-2.33 (m, 1H); 1.49 (s, 3H); 1.46 (s, 9H).

### Preparation B: (R)-tert-butyl 4-bromo-2-methyl-2-(methylsulfonyl)butanoate

The compound of Preparation A (69 g) was separated by semi-preparative chiral HPLC Method A (Hept-EtOH 9-1; flow rate: 34 mL/min; UV detection at 220 nM); the respective retention times (flow rate: 0.8 mL/min) were 9.1 and 10.5 min. The title (R)-enantiomer, identified as the second eluting compound, was obtained as a colourless oil (29.5 g).

¹NMR (d6-DMSO) δ: 3.71-3.63 (m, 1H); 3.45-3.37 (m, 1H); 3.12 (s, 3H); 2.72-2.62 (m, 1H); 2.43-2.33 (m, 1H); 1.49 (s, 3H); 1.46 (s, 9H).

### Preparation C: 2-(RS)-4-(6-bromo-2-oxobenzo[d]oxazol-3(2H)-yl)-2-methyl-2-(methylsulfonyl)-N-(((RS)-tetrahydro-2H-pyran-2-yl)oxy)butanamide

### C.i. (2RS)-tert-butyl 4-(6-bromo-2-oxobenzo[d]oxazol-3(2H)-yl)-2-methyl-2-(methylsulfonyl)butanoate

To a solution of the compound of Preparation A (0.348 g; 1.1mmol) and 6-bromobenzo[d]oxazol-2(3H)-one (prepared as described in EP1988077, 0.214 g; 1 mmol) in DMF (4.2 mL) was added K₂CO₃ (0.212g, 1.53 mmol). The reaction proceeded at 60° C. for 16 h. Water (20 mL) was added and the resulting mixture was extracted three times with EA (3×20 mL). The evaporation residue was purified by CC (EA-Hept) to afford the title compound (0.396 g; 88% yield) as an orange solid.

¹H NMR (d₆-DMSO) δ: 7.69 (d, J=1.8 Hz, 1H); 7.47 (dd, J=1.8, 8.3 Hz, 1H); 7.30 (d, J=8.3 Hz, 1H); 4.00-3.91 (overlapped m, 1H); 3.91-3.81 (overlapped m, 1H); 3.11 (s, 3H); 2.66-2.56 (m, 1H); 2.19-2.10 (m, 1H); 1.58 (s, 3H); 1.35 (s, 9H).

MS (ESI, m/z): 448.04 [M+H⁺] for C₁₇H₂₂NO₆BrS; t_{R}=0.92 min.

### C.ii. (2RS)-4-(6-bromo-2-oxobenzo[d]oxazol-3(2H)-yl)-2-methyl-2-(methylsulfonyl)butanoic acid

To a mixture of intermediate C.i (0.396 g; 0.88 mmol) in 4N HCl in dioxane (5.5 mL) was added water (0.212 mL). The resulting mixture was stirred for 24 h. The reaction mixture was concentrated to dryness. The residue was co-evaporated twice with Et₂O to afford the crude title compound (0.386 g;>95% yield) as a brown foam.

¹H NMR (d₆-DMSO) δ: 7.68 (d, J=1.8 Hz, 1H); 7.45 (dd, J=1.8, 8.3 Hz, 1H); 7.25 (d, J=8.3 Hz, 1H); 4.02-3.84 (m, 2H); 3.11 (s, 3H); 2.64-2.51 (overlapped m, 1H); 2.22-2.10 (m, 1H); 1.59 (s, 3H).

MS (ESI, m/z): 391.94 [M+H⁺] for C₁₃H₁₄NO₆BrS; t_{R}=0.72 min.

### C.iii. (2RS)-4-(6-bromo-2-oxobenzo[d]oxazol-3(2H)-yl)-2-methyl-2-(methylsulfonyl)-N-(((2RS)-tetrahydro-2H-pyran-2-yl)oxy)butanamide

To a solution of intermediate C.ii (crude, 0.386 g) in DMF (3.6 mL) were added successively HOBT (0.277 g; 1.99 mmol), TEA (0.369 mL; 2.65 mmol), THPONH₂ (0.229 g; 1.92 mmol) and EDC.HCl (0.346 g; 1.79 mmol). The reaction mixture was stirred at rt for 24 h. The reaction mixture was partitioned between water (10 mL) and EA (15 mL). The org. layer was washed with water (10 mL), aq. NaHSO₄ (5% w/w, 10 mL) and sat. aq. NaHCO₃ (10 mL). The evaporation residue afforded the title compound (0.407 g; 94% yield) as a yellowish foam.

¹NMR (d₆-DMSO) δ: (mixture of diastereomers) 11.42 (br. s, 1H); 7.67 (d, J=1.7 Hz, 1H); 7.49-7.44 (m, 1H); 7.26 (dd, J=6.0, 8.3 Hz, 1H); 4.94-4.82 (m, 1H); 4.16-3.96 (overlapped m, 1H); 3.96-3.85 (overlapped m, 1H); 3.83-3.63 (m, 1H); 3.54-3.41 (m, 1H); 3.04 (s, 1.5H); 3.02 (s, 1.5H); 2.74-2.58 (m, 1H); 2.14-2.02 (m, 1H); 1.74-1.60 (overlapped m, 3H); 1.61 (overlapped br s, 3H); 1.58-1.22 (overlapped m, 3H).

MS (ESI, m/z): 491.16 [M+H⁺] for C₁₈H₂₃N₂O₇BrS; t_{R}=0.82 min.

### Preparation D: (2RS)-4-(6-iodo-2-oxobenzo[d]oxazol-3(2H)-yl)-2-methyl-2-(methylsulfonyl)-N-(((2RS)-tetrahydro-2H-pyran-2-yl)oxy)butanamide

### D.i. 6-iodobenzo[d]oxazol-2(3H)-one

To a solution of 6-bromobenzo[d]oxazol-2(3H)-one (0.651 g; 3.04 mmol) in 1,4-dioxane (5 mL) was added trans-N-N′-dimethylcyclohexan-1,2-diamine (0.05 mL; 0.32 mmol), NaI (0.934 g; 6.23 mmol) and then CuI (0.043 g; 0.22 mmol). The reaction mixure was heated at 180° C. under microwave irradiation for 40 min. After cooling, the reaction mixure was diluted in water (15 mL) and EA (20 mL). The aq. layer was extracted three times with EA (3×15 mL). The evaporation residue was purified by CC (Hept-EA) to afford the title compound (0.352 g; 44% yield) as a reddish solid.

¹H NMR (d6-DMSO) δ: 11.78 (s, 1H), 7.68 (d, J=1.5 Hz, 1H), 7.48 (dd, J=1.5, 8.1 Hz, 1H), 6.92 (d, J=8.1 Hz, 1H).

### D.ii. (2RS)-4-(6-iodo-2-oxobenzo[d]oxazol-3(2H)-yl)-2-methyl-2-(methylsulfonyl)-N-(((2RS)-tetrahydro-2H-pyran-2-yl)oxy)butanamide

Starting from the intermediate D.i (0.352 g; 1.65 mmol) and proceeding in analogy to Preparation C, steps C.i (71% yield), step C.ii (>95% yield) and C.iii (97% yield), the title compound (0.322 g) was obtained as a brownish solid.

¹H NMR (d₆-DMSO) δ: (mixture of diastereomers) 11.44 (br. s, 1H); 7.76 (d, J=1.5 Hz, 1H); 7.63-7.59 (m, 1H); 7.15-7.10 (m, 1H); 4.93-4.89 (m, 0.5H); 4.85-4.81 (m, 0.5H); 4.15-4.05 (m, 1H); 3.95-3.84 (m, 1H); 3.80-3.62 (m, 1H); 3.52-3.43 (m, 1H); 3.04 (s, 1.5H); 3.01 (s, 1.5H); 2.71-2.57 (m, 1H); 2.11-2.01 (m, 1H); 1.72-1.48 (overlapped m, 6H); 1.61 (s, 1.5H); 1.60 (s, 1.5H).

### Preparation E: (2R)-4-(6-ethynyl-2-oxobenzo[d]oxazol-3(2H)-yl)-2-methyl-2-(methylsulfonyl)-N—(((RS)-tetrahydro-2H-pyran-2-yl)oxy)butanamide

### E.i. (2RS)-4-(6-iodo-2-oxobenzo[d]oxazol-3(2H)-yl)-2-methyl-2-(methylsulfonyl)-N-(((2RS)-tetrahydro-2H-pyran-2-yl)oxy)butanamide

Starting from the intermediate D.i (9.45 g; 44.2 mmol) and the compound of Preparation B (15.31 g; 48.6 mmol) and proceeding in analogy to Preparation C, steps C.i (55% yield), C.ii (84% yield) and C.iii (86% yield), the title compound (9.24 g) was obtained, after purification by CC (Hept-EA) as a white solid.

¹H NMR (d₆-DMSO) δ: (mixture of diastereomers) 11.44 (br. s, 1H); 7.76 (m, 1H); 7.62 (m, 1H); 7.13 (m, 1H); 4.92 (m, 0.5H); 4.84 (m, 0.5H); 4.11 (m, 0.5H); 4.03 (m, 0.5H); 3.92 (m, 1H); 3.77 (m, 0.5H); 3.69 (m, 0.5H); 3.50 (m, 1H); 3.05 (s, 1.5H); 3.03 (s, 1.5H); 2.65 (m, 1H); 2.08 (m, 1H); 1.71-1.64 (overlapped m, 3H); 1.62 (s, 1.5H); 1.61 (s, 1.5H);1.58-1.49 (m, 3H).

MS (ESI, m/z): 538.92 [M+H⁺] for C₁₈H₂₃N₂O₇IS; t_{R}=0.83 min.

### E.ii. (2R)-2-methyl-2-(methylsulfonyl)-4-(2-oxo-6-((trimethylsilyl)ethynyl)benzo[d]oxazol-3(2H)-yl)-N-(2RS)-tetrahydro-2H-pyran-2-yl)oxy)butanamide

To a mixture of CuI (0.670 g, 3.52 mmol) and intermediate E.i (9.240 g; 17.6 mmol) in degassed THF (129 mL) was added added PdCl₂(PPh₃)₂ (1.235 g; 1.76 mmol). Trimethylsilylacetylene (3.76 mL; 26.4 mmol) and degassed TEA (7.36 mL, 52.8 mmol) were added. The mixture was stirred at rt for 20 min. The reaction mixture was concentrated in vacuo and the residue was purified by CC (Hept-EA) to afford the title compound (7.54 g; 84% yield) as a beige foam.

¹H NMR (d6-DMSO) δ: 11.44 (br. s, 1H); 7.46 (m, 1H); 7.38 (m, 1H); 7.28 (m, 1H); 4.92 (m, 0.5H); 4.86 (m, 0.5H); 4.11 (m, 0.5H); 4.03 (m, 0.5H); 3.92 (m, 1H); 3.77 (m, 0.5H); 3.70 (m, 0.5H); 3.49 (m, 1H); 3.05 (s, 1.5H); 3.03 (s, 1.5H); 2.65 (m, 1H); 2.08 (m, 1H); 1.72-1.64 (m, 3H); 1.62 (s, 1.5H); 1.61 (s, 1.5H); 1.58-1.48 (m, 3H); 0.23 (s, 9H).

MS (ESI, m/z): 509.04 [M+H⁺] for C₂₃H₃₂N₂O₇SSi; t_{R}=0.94 min.

### E.iii. (2R)-4-(6-ethynyl-2-oxobenzo[d]oxazol-3(2H)-yl)-2-methyl-2-(methylsulfonyl)-N-(((2RS)-tetrahydro-2H-pyran-2-yl)oxy)butanamide

To a solution of intermediate E.ii (7.540 g, 14.8 mmol) in THF (31.3 mL) was added TBAF (1M in THF, 31.3 mL; 31.3 mmol). The mixture was stirred for 15 min under nitrogen. The solvent was evaporated and the residue was purified by CC (DCM-MeOH) to afford the title compound (4.28 g; 66% yield) as an off-white foam.

¹H NMR (d6-DMSO) δ: 11.44 (br. s, 1H); 7.49 (d, J=1.2 Hz, 1H); 7.41 (m, 1H); 7.28 (t, J=8.5 Hz, 1H); 4.92 (m, 0.5H): 4.86 (m, 0.5H); 4.11 (m, 0.5H); 4.03 (m, 0.5H); 3.92 (m, 1H); 3.78 (m, 0.5H); 3.71 (m, 0.5H); 3.48 (m, 1H); 3.05 (s, 1.5H); 3.02 (s, 1.5H); 2.65 (m, 1H); 2.08 (m, 1H); 1.71-1.63 (m, 3H); 1.62 (s, 1.5H); 1.61 (s, 1.5H); 1.57-1.50 (m, 3H).

MS (ESI, m/z): 437.0 [M+H⁺] for C₂₀H₂₄N₂O₇S; t_{R}=0.75 min.

### Preparation F: 1-(bromoethynyl)cyclopropan-1-amine hydrochloride

### F.i. Tert-butyl (1-formylcyclopropyl)carbamate

To a solution of tert-butyl (1-(hydroxymethyl)cyclopropyl)carbamate (commercial, 15 g; 80.3 mmol) in DCM (235 mL), cooled to −20° C., was slowly added DIPEA (45 mL; 263 mmol) over 15 min. A solution of Pyr.SO₃ (38.75 g; 110 mmol) in DMSO (108 mL; 1.52 mol) was added dropwise over 45 min. The reaction mixture was stirred at 0° C. for 2 h45. The reaction mixture was partitioned between water (1 L) and DCM (200 mL). The two layers were separated and the aq. layer was extracted once more with DCM (300 mL). The evaporation residue was purified by CC (Hept-EA) to afford the title compound (13.18 g; 89% yield) as a white solid.

¹H NMR (d6-DMSO) δ: 8.99 (s, 1H); 7.55 (s, 1H); 1.44-1.31 (overlapped m, 2H); 1.39 (s, 9H); 1.19-1.10 (m, 2H).

MS (ESI, m/z): 186.2 [M+H⁺] for C₉H₁₅NO₃; t_{R}=0.62 min

### F.ii. Tert-butyl (1-(2,2-dibromovinyl)cyclopropyl)carbamate

To a solution of CBr₄ (18.3g, 54.8 mmol) in DCM (40 mL) cooled at −20° C., was added dropwise over 1 h a solution of triphenylphosphine (29.6 g; 107 mmol) in DCM (65 mL). The solution was allowed to slowly warm to 0° C. and then cooled to −78° C. TEA (7.5 mL; 53.9 mmol) was added. A solution of intermediate F.i (5.0 g, 26.9 mmol) in DCM (50 mL) was added dropwise over 45 min at −78° C. The IT was kept under −72° C. The suspension was kept stirring at this temperature for 30 min before warming to 15° C. The mixture was diluted in Et₂O (20 mL), and the solids were filtered off. The filtrate was concentrated to dryness and the residue was purified by CC (EA-Hept) to afford the title compound (7.7 g, 84% yield) as a white solid.

¹H NMR (d6-DMSO) δ: 7.46 (s, 1H); 6.48 (s, 1H); 1.37 (s, 9H); 0.97-0.94 (m, 2H); 0.92-0.89 (m, 2H).

### F.iii. Tert-butyl(1-(bromoethynyl)cyclopropyl)carbamate

A solution of intermediate F.ii (1.5 g; 4.4 mmol) in dry THF (10 mL) cooled at −78° C., was treated dropwise over 1 h 30, with a freshly prepared suspension of t-BuOK (2.71 g; 24.2 mmol) in dry THF (24.2 mL). The IT was kept under −73° C. The mixture was stirred for 2 h at −75° C. The suspension was slowly allowed to warm to 0° C. and the mixture was stirred a this temperature for 1 h. Brine (50 mL) was added slowly over 2 min. The IT increased to 9° C. The mixture was allowed to reach rt. Et₂O (60 mL) was added. The aq. layer was separated and extracted with Et₂O (70 mL). The combined org. layers were washed with brine (30 mL), dried over Na₂SO₄, filtered and concentrated to afford the title compound (1.08 g; 95% yield) as a white solid.

¹H NMR (d6-DMSO) δ: 7.61 (s, 1H); 1.38 (s, 9H); 1.07-1.03 (m, 2H); 0.95-0.91 (m, 2H).

### F.iv. 1-(bromoethynyl)cyclopropan-1-amine hydrochloride

A solution of intermediate F.iii (2.66 g; 10.2 mmol) in 4N HCl in dioxane (10.2 mL) was stirred for 1 h. The reaction mixture was evaporated and the residue was triturated with Et₂O (15 mL), filtered and the solid was dried in vacuo to afford the title compound (1.97 g; 84% yield) as a white solid.

¹H NMR (d6-DMSO) δ: 8.94 (s, 2H); 1.34-1.27 (m, 2H); 1.27-1.20 (m, 2H).

### Preparation G: 1-(bromoethynyl)-N-methylcyclopropan-1-amine hydrochloride

### G.i. Tert-butyl(1-(((tert-butyldiphenylsilyl)oxy)methyl)cyclopropyl)carbamate

To a solution of tert-butyl (1-(hydroxymethyl)cyclopropyl)carbamate (3.5 g; 18.7 mmol) and imidazole (2.54 g; 37.4 mmol) in DCM (40 mL) was added TBDPSC1 (4.11 mL; 18.7 mmol). The reaction mixture was stirred for 4 h. Water (50 mL) and DCM (20 mL) were added. The two layers were separated and the aq. phase was extracted twice with DCM (2×25 mL).The evaporation residue was purified by CC (EA-Hept) to afford the title compound (8.85 g; >95% yield) as a colorless oil.

¹H NMR (d6-DMSO) δ: 7.64-7.60 (m, 4H); 7.49-7.40 (m, 6H); 7.20 (s, 1H); 3.66 (s, 2H); 1.36 (br. s, 9H); 1.00 (s, 9H); 0.71-0.65 (m, 2H); 0.64-0.60 (m, 2H).

MS (ESI, m/z): 426.1 [M+H⁺] for C₂₅H₃₅NO₃Si; t_{R}=1.11 min.

### G.ii. Tert-butyl(1-(((tert-butyldiphenylsilyl)oxy)methyl)cyclopropyl)(methyl)carbamate

A suspension of NaH (60% in oil dispersion, 1.33 g; 33.2 mmol) in dry DMF (21 mL) was added dropwise to an ice-chilled solution of intermediate Gi (7.85 g; 18.4 mmol) in dry DMF (13 mL). The reaction mixture was stirred for 30 min then MeI (1.38 mL; 22.1 mmol) was added dropwise. After 3 h stirring at rt, water (200 mL) was added carefully and the resulting suspension was extracted with EA (2×100 mL). The evaporation residue was purified by CC (Hept-EA) to afford the title compound (5.78 g; 71% yield) as a white solid.

MS (ESI, m/z): 440.1 [M+H⁺] for C₂₆H₃₇NO₃Si; t_{R}=1.15 min.

### G.iii. 1-(bromoethynyl)-N-methylcyclopropan-1-amine hydrochloride

Starting from the intermediate G.ii (6.57 g; 14.9 mmol), and proceeding sucessively in analogy to Preparation E, step E.iii (97% yield) and Preparation F, steps F.i (91% yield), F.ii (91% yield), F.iii (98% yield) and F.iv (98% yield), the title compound (2.4 g) was obtained, after final trituration in Et₂O, as a white solid.

¹H NMR (d6-DMSO) δ: 9.73 (s, 2H), 2.65 (s, 3H), 1.46-1.42 (m, 2H), 1.29-1.24 (m, 2H).

MS (ESI, m/z): 173.99 [M+H⁺] for C₆H₈NBr; t_{R}=0.35 min.

### Preparation H: ((1S,2S)-2-(bromoethynyl)cyclopropyl)methanol

### H.i. ((1S*,2S*)-2-(2,2-dibromovinyl)cyclopropyl)methyl acetate

To a solution of CBr₄ (30.0 g; 88.9 mmol) in DCM (60 mL) cooled at −20° C., was added dropwise over 45 min a solution of PPh₃ (45.8 g, 175 mmol) in DCM (100 mL). The mixture was kept stirred at this temperature for 30 min and then cooled to −78° C. A solution of ((1S*,2S*)-2-formylcyclopropyl)methyl acetate (6.18 g, 43.5 mmol, prepared as described in WO 2012/154204) in DCM (80 mL) was added dropwise over 45 min, keeping the IT below −70° C. The mixture was stirred at this temperature for 30 min and allowed to warm to rt over 1 h. The solvent was removed in vacuo and the residue was purified by CC (EA-Hept) to afford the title acetate as a clear oil (4.84 g; 37% yield).

¹H NMR (CDCl₃) δ: 5.84 (d, J=9.0 Hz, 1H); 3.97 (m, 2H); 2.07 (s, 3H); 1.61 (m, 1H); 1.33 (m, 1H); 0.92-0.78 (m, 2H).

MS (ESI, m/z) : 295.0 [M+H⁺] for C₈H₁₀O₂Br₂; t_{R}=0.87 min.

### H.ii. ((1S,2S)-2-(bromoethynyl)cyclopropyl)methyl acetate and ((1R,2R)-2-(bromoethynyl)cyclopropyl)methyl acetate

To a solution of intermediate H.i (3.94 g; 13.2 mmol) in THF (75 mL) was added TBAF trihydrate (23.2 g; 72.8 mmol). The reaction mixture was heated at 60° C. for 4 h. The reaction mixture was cooled to rt and diluted with diethyl ether (150 mL). The org. phase was washed with water (60 mL) and brine (60 mL), dried over MgSO₄ and concentrated to dryness. The residue was purified by CC (EA-Hept) to afford the title compound as a yellow oil (1.76 g, 61% yield). The racemic product was separated by semi-preparative chiral HPLC Method A (Hept-EtOH 9-1; flow rate: 20 mL/min, UV detection at 223 nm), the respective retention times (flow rate: 0.8 mL/min) were 5.9 and 8.7 min. The title enantiomers were obtained as clear oils (0.64 g each).

### First-Eluting Enantiomer, (1S,2S)-Configurated:

¹H NMR (CDCl₃) δ: 3.97 (dd, J=6.5, 11.7 Hz, 1H); 3.84 (dd, J=7.5, 11.7 Hz, 1H); 2.06 (s, 3H); 1.50 (m, 1H); 1.25 (m, 1H); 0.97 (m, 1H); 0.76 (m, 1H).

[α]_{D}=+96° (c=1.03; MeOH).

### Second-Eluting Enantiomer, (1R,2R)-Configurated:

¹H NMR (CDCl₃) δ: 3.97 (dd, J=6.5, 11.7 Hz, 1H); 3.84 (dd, J=7.5, 11.7 Hz, 1H); 2.06 (s, 3H); 1.50 (m, 1H); 1.25 (m, 1H); 0.97 (m, 1H); 0.76 (m, 1H).

[α]_{D}=−94° (c=1.01; MeOH).

The respective absolute configurations of these compounds have been determined though transformation of the second-eluting enantiomer into the corresponding (S) and (R) α-methoxy-α-trifluoromethylphenylacetyl esters and the subsequent analysis of their NMR spectra as described by Tsuda et al. in Chem. Pharm. Bull. (2003), 51, 448-451.

### H.iii. (1S,2S)-2-(bromoethynyl)cyclopropyl)methanol

To a solution of (1S,2S)-configurated intermediate H.ii (1 g, 4.61 mmol) in MeOH (22.9 mL) was added K₂CO₃ (1.273 g, 9.21 mmol). The suspension was stirred for 2 h. The solvent was evaporated under reduced pressure. The residue was diluted in DCM-MeOH 9-1 (100 mL) and washed with 15% aq. NaHSO4 solution (30 mL). The aq. layer was extracted with DCM-MeOH 9-1 (2×50 mL). The combined org. layers were dried over MgSO₄, filtered and concentrated to dryness to afford the title compound (0.803 g, >95% yield) as a colorless oil.

¹H NMR (d6-DMSO) δ: 4.63 (t, J=5.7 Hz, 1H); 3.37 (m, 1H); 3.18 (m, 1H); 1.29-1.20 (m, 2H); 0.76 (m, 1H); 0.70 (ddd, J=4.2, 6.0, 8.5 Hz, 1H).

### Preparation I: ((1R,2R)-2-(bromoethynyl)-1-fluorocyclopropyl)methyl benzoate

### I.i ((1R*,2R*)-2-(((tert-butyldiphenylsilyl)oxy)methyl)-1-fluorocyclopropyl)methanol

To a solution of ethyl (1R*,2R*)-2-(((tert-butyldiphenylsilypoxy)methyl)-1-fluorocyclopropane-1-carboxylate (0.5 g; 1.25 mmol; prepared as described in Sakagami et al., Bioorg. Med. Chem. (2008), 16(8), 4359-4366) in THF (9 mL), cooled to −78° C., was added dropwise LiBH₄ (2A/in THF; 2.2 mL; 4.4 mmol). The reaction mixture was allowed to reach rt and stirred at rt for 24 h. MeOH (2 mL) was carefully added, the reaction mixture was stirred for 20 min, concentrated to dryness and partitioned between water (10 mL) and DCM (15 mL). The aq. layer was extracted with DCM (2×10 mL). The combined org. layers were dried over Na₂SO₄ and filtered. After concentration of the filtrate to dryness, the title compound was obtained as a colourless oil (0.429 g; 96% yield).

¹H NMR (CDCl₃) δ: 7.72-7.60 (m, 4H); 7.45-7.36 (m, 6H); 3.89 (ddd, J=1.6, 6.0, 11.0 Hz, 1H); 3.83-3.80 (m, 1H); 3.78-3.70 (m, 2H); 1.74 (t, J=6.4 Hz, 1H); 1.33-1.24 (m, 1H); 1.05 (s, 9H); 0.88-0.79 (m, 2H).

MS (ESI, m/z): 358.95 [M+H⁺] for C₂₁H₂₇O₂FSi; t_{R}=1.01 min.

### I.ii ((1R*,2R*)-2-(((tert-butyldiphenylsilyl)oxy)methyl)-1-fluorocyclopropyl)methyl benzoate

To a solution of intermediate I.i (5.51 g, 15.4 mmol) in THF (93 mL) was added TEA (6 mL; 43.1 mmol). Benzoyl chloride (3.6 mL; 30.7 mmol) was added dropwise over 2 min at 0° C. The reaction mixture was stirred at 0° C. for 5 h before being poured onto water (75 mL). The aq. layer was extracted with EA (3×50 mL). The combined org. layers were dried over MgSO₄ and concentrated to dryness. The residue was purified by CC (Hept-EA) to afford the title compound as a colourless oil (6.49 g; 91% yield).

¹H NMR (CDCl₃) δ: 8.12-8.09 (m, 2H); 7.70-7.67 (m, 4H); 7.56 (m, 1H); 7.44-7.40 (m, 4H); 7.38-7.35 (m, 4H); 4.62 (m, 1H); 4.51 (ddd, J=1.1, 13.0, 23.8 Hz, 1H); 3.93 (ddd, J=1.5, 5.6, 11.0 Hz, 1H); 3.70 (ddd, J=1.1, 8.4, 10.9 Hz, 1H); 1.46 (m, 1H); 1.30 (m, 1H); 1.02 (s, 7H); 0.97 (m, 1H); 0.91-0.84 (m, 2H).

MS (ESI, m/z): 463.07 [M+H^{I}] for C₂₈H₃₁O₃FSi ; t_{R}=1.14 min.

### I.iii. ((1R*,2R*)-1-fluoro-2-(hydroxymethyl)cyclopropyl)methyl benzoate

To a solution of intermediate I.ii (6.49 g; 14 mmol) in THF (26 mL) was added TBAF (1M in THF, 17 mL). The reaction mixture was stirred at rt for 45 min. The reaction mixture was concentrated in vacuo and the residue was purified by CC (DCM-MeOH) to afford the title compound (2.81 g; 89% yield) as a yellow oil.

¹H NMR (CDCl₃) δ: 8.10-8.08 (m, 2H), 7.58 (m, 1H), 7.48-7.45 (m, 2H), 4.64 (m, 1H), 4.55 (m, 1H), 3.97 (ddd, J=1.5, 5.8, 11.8 Hz, 1H), 3.68 (ddd, J=1.4, 8.7, 11.8 Hz, 1H), 1.52 (m, 1H), 1.12-1.04 (m, 2H).

### I.iv. ((1R,2R)-2-(2,2-dibromovinyl)-1-fluorocyclopropyl)methyl benzoate

Starting from intermediate I.iii (2.77 g; 12.4 mmol) and proceeding successively in analogy to Preparation F, steps F.i (84% yield), F.ii (adding 2 eq. TEA, 77% yield), a mixture of enantiomers (2.71 g) was obtained. After separation by chiral prep-HPLC (Method B) (Hept-EtOH 3-7; flow rate: 16 mL/min, UV detection at 224 nm), the title enantiomer (first-eluting enantiomer) was obtained as a white solid (1.25 g). The retention time on analytical chiral HPLC (Hept-EtOH 3-7; flow rate: 0.8 mL/min) was 5.3 min.

¹H NMR (d6-DMSO) δ: 8.01-7.99 (m, 2H); 7.69 (m, 1H); 7.58-7.54 (m, 2H); 6.38 (dd, J=1.4, 8.9 Hz, 1H); 4.75-4.57 (m, 2H); 2.09 (m, 1H); 1.55-1.48 (m, 2H).

### I.v. ((1R,2R)-2-(bromoethynyl)-1-fluorocyclopropyl)methyl benzoate

To a solution of intermediate I.iv (2.05 g, 5.42 mmol) in THF (20 mL) was added TBAF (1M in THF, 22 mL; 21.7 mmol). The mixture was stirred over night. The reaction mixture was diluted with EA (50 mL) and water (30 mL). The two layers were separated and the org. layer was extracted with EA (3×50 mL). The evaporation residue was purified by CC (Hept-EA) to afford the title compound (1.1 g; 68% yield) as a yellowish oil.

¹H NMR (d6-DMSO) δ: 8.03-7.99 (m, 2H); 7.70 (m, 1H); 7.60-7.55 (m, 2H); 4.67-4.51 (m, 2H); 2.09-2.04 (m, 1H); 1.49-1.37 (m, 2H).

### Preparation J: 3-bromoprop-2-yn-1-yl 3-hydroxyazetidine-1-carboxylate

### J.i. 3-bromoprop-2-yn-1-yl(2,5-dioxopyrrolidin-1-yl)carbonate

To a solution of 3-bromoprop-2-yn-1-ol (1 g; 7.41 mmol) in MeCN (85 mL) was added TEA (2.1 mL; 14.8 mmol) and DSC (6.0 g; 22.2 mmol). The reaction mixture was stirred for 30 min. The reaction mixture was diluted with EA (100 mL)and washed with 5% aq. citric acid aq. (3×50 mL). The evaporation residue was purified by CC (Hept-EA) to afford the title product (1.38 g; 67% yield) as a beige solid.

¹H NMR (d6-DMSO) δ: 5.13 (s, 2H); 2.83 (s, 4H).

### J.ii. 3-bromoprop-2-yn-1-yl 3-hydroxyazetidine-1-carboxylate

To a solution of intermediate J.i (1.38 g; 5 mmol) in DCM (65 mL) were added 3-hydroxyazetidine hydrochloride (0.559 g, 5 mmol) and TEA (1.39 mL; 10 mmol). After 45 min. stirring, the reaction mixture was diluted in DCM (200 mL), washed with sat. NaHCO₃ (3×200 mL). The evaporation residue was purified by CC (Hept-EA) to afford the title compound (0.875 g) as a white solid.

¹H NMR (d6-DMSO) δ: 5.35 (m, 1H); 4.26-4.22 (m, 2H); 3.88 (d, J=8.4 Hz, 2H); 2.82 (s, 4H); 1.39 (m, 9H).

### Preparation K: 3-(bromoethynyl)azetidine hydrochloride

### K.i. Tert-butyl 3-(bromoethynyl)azetidine-1-carboxylate

To a stirring solution of tert-butyl 3-ethynylazetidine-1-carboxylate (prepared as described in WO 2014/165075, 9 g; 49.4 mmol) in acetone (200 mL) were added successively NBS (10.6 g; 59.5 mmol) and AgNO₃ (0.90 g; 5.27 mmol). The mixture was stirred for 2 h. After evaporation to dryness, the residue was purified by CC (Hex-TBME) to afford the title compound (11.8 g, 91% yield) as a colourless oil.

¹H NMR (CDCl₃) δ: 4.14 (m, 2H); 3.96 (dd, J=6.3, 8.4 Hz, 2H); 3.34 (m, 1H); 1.46 (s, 9H).

### K.ii. 3-(bromoethynyl)azetidine hydrochloride

Starting from the intermediate K.i (11.77 g, 45.2 mmol) and proceeding in analogy to Preparation F, step F.iv, the title compound was obtained, after trituration in Et₂O, as an off-white solid (8.7 g; >95% yield).

¹H NMR (CDCl₃) δ: 9.10-9.44 (m, 2H), 4.15-4.06 (m, 2H), 3.96-3.87 (m, 2H), 3.74 (m, 1H).

MS (ESI, m/z): 162.0 [M+H⁺] for C₅H₆NBr; t_{R}=0.23 min.

### Preparation L: (2R)-4-(6-ethynyl-2-oxobenzo[d]thiazol-3(2H)-yl)-2-methyl-2-(methylsulfonyl)-N-((2RS)-(tetrahydro-2H-pyran-2-yl)oxy)butanamide

### L.i. 6-iodobenzo[d]thiazol-2(3H)-one

To a mixture of 6-bromobenzo[d]thiazol-2-(3H)-one (12.500 g; 54.3 mmol), CuI (2.069 g; 10.9 mmol) and NaI (16.286 g, 109 mmol) were added dioxane (70 mL) and trans-N,N′-dimethylcyclohexan-1,2-diamine (3.43 mL; 21.7 mmol). The reaction mixture was refluxed for 48 h. After cooling, EA (200 mL) and water (200 mL) were added. The two layers were separated and the aq. layer was extracted with EA (2×200 mL). The concentration residue was purified by CC (Hept-EA) to afford the title product (4.19 g; 28% yield) as a beige solid.

¹H NMR (d6-DMSO) δ: 8.08 (s, 1H); 7.74 (d, J=8.6 Hz, 1H); 7.18 (d, J=8.5 Hz, 1H); 4.11 (m, 1H); 4.06-3.99 (m, 3H); 3.11 (s, 3H); 2.57 (m, 1H); 2.10 (m, 1H); 1.64 (s, 3H); 1.14 (t, J=7.1 Hz, 3H).

### L.ii. (2R)-ethyl 4-(6-iodo-2-oxobenzo[d]thiazol-3(2H)-yl)-2-methyl-2-(methylsulfonyl)butanoate

Starting from the intermediate L.i (4.1 g; 15.1 mmol) and (R)-ethyl 4-bromo-2-methyl-2-(methylsulfonyl)butanoate (4.4 g; 15.3 mmol) and proceeding in analogy to Preparation C, step C.i (79% yield), the title compound (5.77 g) was obtained, after purification by CC (Hept-EA), as a beige solid.

¹H NMR (d6-DMSO) δ: 8.08 (s, 1H); 7.74 (d, J=8.6 Hz, 1H); 7.18 (d, J=8.5 Hz, 1H); 4.11 (m, 1H); 4.06-3.99 (m, 3H); 3.11 (s, 3H); 2.57 (m, 1H); 2.10 (m, 1H); 1.64 (s, 3H); 1.14 (t, J=7.1 Hz, 3H).

MS (ESI, m/z): 483.85 [M+H⁺] for C₁₅H₁₈NO₅IS₂; t_{R}=0.90 min.

### L.iii. (2R)-4-(6-iodo-2-oxobenzo[d]thiazol-3(2H)-yl)-2-methyl-2-(methylsulfonyl)butanoic acid

To a solution of intermediate L.ii (5.77 g; 11.9 mmol) in a THF-MeOH-water mixture (2-2-1, 125 mL) was added in one portion LiOH.H₂O (1.845 g; 24.6 mmol). The reaction mixure was stirred for 1 h. The volatiles were removed under reduced pressure. The pH of the aq. layer was adjusted to 4 by addition of 10% NaHSO₄ solution. The resulting precipitate was filtered, scarcely washed with water and dried under reduced pressure to a constant weight. The title compound (5.38 g, 99% yield) was obtained as a white solid.

¹H NMR (d6-DMSO) δ: 8.06 (s, 1H); 7.69 (d, J=8.5 Hz, 1H); 7.43 (d, J=8.5 Hz, 1H); 4.10 (m, 1H); 3.97 (m, 1H); 3.08 (s, 3H); 2.35 (m, 1H); 1.97 (m, 1H); 1.46 (s, 3H).

MS (ESI, m/z): 455.71 [M+H⁺] for C₁₃H₁₄NO₅IS₂; t_{R}=0.78 min.

### L.iv. (2R)-4-(6-iodo-2-oxobenzo[d]thiazol-3(2H)-yl)-2-methyl-2-(methylsulfonyl)-N-((2RS)-(tetrahydro-2H-pyran-2-yl)oxy)butanamide

Starting from the intermediate L.iii (5.38 g; 11.8 mmol) and proceeding in analogy to Preparation C, step C.iii (73% yield), the title compound (4.78 g), was obtained, without purification, as a beige foam.

¹H NMR (d6-DMSO) δ: (mixture of stereoisomers) 11.46 (br. s, 1H); 8.08 (s, 1H); 7.74 (d, J=8.4 Hz, 1H); 7.16 (m, 1H); 4.98 (m, 0.5H); 4.95 (m, 0.5H); 4.14 (m, 0.5H); 4.09-3.97 (m, 1.5H); 3.82 (m, 0.5H); 3.72 (m, 0.5H); 3.51 (m, 1H); 3.04 (s, 1.5H); 3.02 (s, 1.5H); 2.55 (m, 1H); 1.98 (m, 1H); 1.74-1.66 (m, 3H); 1.64-1.60 (m, 3H); 1.58-1.50 (m, 3H).

MS (ESI, m/z): 554.86 [M+H⁺] for C₁₈H₂₃N₂O₆IS₂; t_{R}=0.87 min.

### L.v. (2R)-4-(6-ethynyl-2-oxobenzo[d]thiazol-3(2H)-yl)-2-methyl-2-(methylsulfonyl)-N-((2RS)-(tetrahydro-2H-pyran-2-yl)oxy)butanamide

Starting from the intermediate L.iv (4.78 g; 8.62 mmol) and proceeding successively in analogy to Preparation E, steps E.ii (69% yield) and E.iii (91% yield), the title compound (2.46 g), was obtained after purification by CC (DCM-MeOH), as a beige foam.

MS (ESI, m/z): 452.99 [M+H⁺] for C₂₀H₂₄N₂O₆S₂; t_{R}=0.83 min.

### Preparation M: (1R,2R)-2-(bromoethynyl)-1-fluorocyclopropyl)methanol

Starting from the compound of Preparation I (0.6 g; 2 mmol) and proceeding in analogy to Preparation H, step H.iii (88% yield), the title compound (0.344 g) was obtained, after purification by CC (Hept-EA) as a yellowish oil.

¹H NMR (d6-DMSO) δ: 5.17 (t, J=6.0 Hz, 1H); 3.68-3.52 (m, 2H); 1.72 (m, 1H); 1.28-1.15 (m, 2H).

### Preparation N: (1S,2S)-2-(bromoethynyl)cyclopropylnnethyl di-tert-butyl phosphate

### N.i. ((1S,2S)-2-(bromoethynyl)cyclopropyl)methanol

To a solution of ((1S,2S)-2-(bromoethynyl)cyclopropyl)methyl acetate (prepared as described in WO2005/036964, 1 g, 4.61 mmol) in MeOH (22.9 mL) was added K₂CO₃ (1.273 g, 9.21 mmol). The suspension was stirred 30 min. The solvent was evaporated under reduced pressure. The residue was diluted in DCM-MeOH (9-1, 100 mL) and the org. layer washed with aq. 15% NaHSO₄ solution (30 mL).and dried over MgSO₄, filtered and concentrated to dryness to afford the title product (0.803 g, quant.) as a colorless oil.

¹H NMR (d6-DMSO) δ: 4.63 (t, J=5.7 Hz, 1H); 3.37 (m, 1H); 3.19 (m, 1H); 1.29-1.20 (m, 2H); 0.76 (m, 1H); 0.70 (ddd, J=4.2, 6.0, 8.5 Hz, 1H).

### N.ii. ((1S,2S)-2-(bromoethynyl)cyclopropyl)methyl di-tert-butyl phosphate

To a solution of intermediate N.i (0.570 g; 3.26 mmol) in THF (5 mL) cooled to 0° C. was added portionwise NaH (60% in mineral oil, 0.195 g; 4.89 mmol). The mixture was stirred at 0° C. for 5 min and at rt for 1 h. After cooling to 0° C., di-tert-butyl phosphorochloridate (prepared as described in WO 2010/32147, 1.043 g; 4.56 mmol) was added dropwise. The mixture was stirred for 5 h. EA (50 mL) and water (50 mL) were added. The two layers were separated and the aq. layer was extracted with EA (50 mL). The evaporation residue was purified by CC (Hept-EA) to afford the title compound (0.638 g; 53% yield) as a light yellow oil.

¹H NMR (d6-DMSO) δ: 8.47 (br. s, 2H); 3.57 (s, 1H); 3.19-3.11 (m, 2H); 3.09-3.00 (m, 2H); 1.96-1.89 (m, 2H); 1.88-1.80 (m, 2H).

MS (ESI, m/z): 366.91 [M+H⁺] for C₁₄H₂₄O₄BrP; t_{R}=0.92 min.

### REFERENCE EXAMPLES

### Reference Example 1

### (2RS)-4-(6-(2-fluoro-4-methoxyphenyl)-2-oxobenzo[d]oxazol-3(2H)-yl)-N-hydroxy-2-methyl-2-(methylsulfonyl)butanamide

Starting from the compound of Preparation C (0.202 g; 0.41 mmol) and 2-fluoro-4-methoxyphenylboronic acid (0.077 g; 0.44 mmol) and proceeding successively in analogy to Procedure A (>95% yield) and Procedure B (47% yield), the title compound was obtained, after precipitation in water and trituration in Et₂O, as an off-white solid (0.087 g).

¹H NMR (d6-DMSO) δ: 11.06 (d, J=1.5 Hz, 1H); 9.28 (d, J=1.5 Hz, 1H); 7.50-7.42 (m, 2H); 7.40-7.33 (m, 2H); 6.98-6.85 (m, 2H); 4.00-3.90 (m, 1H); 3.81 (s, 3H); 3.76-3.66 (m, 1H); 3.05 (s, 3H); 2.73-2.63 (m, 1H); 2.14-2.03 (m, 1H); 1.61 (s, 3H).

MS (ESI, m/z): 453.10 [M+H⁺] for C₂₀H₂₁N₂O₇FS₂; t_{R}=0.77 min.

### Reference Example 2

### (2RS)—N-hydroxy-4-(6-((3-hydroxyoxetan-3-yl)buta-1,3-diyn-1-yl)-2-oxobenzo[d]oxazol-3(2H)-yl)-2-methyl-2-(methylsulfonyl)butanamide

Starting from the compound of Preparation C (0.074 g; 0.17 mmol) and 3-(iodoethynyl)oxetan-3-ol (prepared as described in WO 2005/036964, 0.041 g; 0.18 mmol) and proceeding successively in analogy to Procedure C (>84% yield) and Procedure B (19% yield), the title compound was obtained, after purification by prep-HPLC (Method 1), as a brownish solid (0.012 g).

¹H NMR (d6-DMSO) δ: 11.06 (br. s, 1H), 9.20 (br. s, 1H), 7.61 (s, 1H), 7.51 (d, J=8.1 Hz, 1H), 7.31 (d, J=8.1 Hz, 1H), 6.73 (br. s, 1H); 4.69 (d, J=6.6 Hz, 2H), 4.54 (d, J=6.6 Hz, 2H); 3.91 (m, 1H), 3.67 (m, 1H), 3.00 (s, 3H), 2.67 (m, 1H), 2.04 (m, 1H), 1.57 (s, 3H).

MS (ESI, m/z): 449.2 [M+H⁺] for C₂₀H₂₀N₂O₈S; t_{R}=0.64 min.

The racemic mixtures of Reference Examples 1 to 2 can be separated into their enantiomers using, for example, semipreparative chiral HPLC. Thus, the following further invention compounds would be obtained:

(2R)-4-(6-(2-fluoro-4-methoxyphenyl)-2-oxobenzo[d]oxazol-3(2H)-yl)-N-hydroxy-2-methyl-2-(methylsulfonyl)butanamide;

(2R)—N-hydroxy-4-(6-((3-hydroxyoxetan-3-yl)buta-1,3-diyn-1-yl)-2-oxobenzo[d]oxazol-3(2H)-yl)-2-methyl-2-(methylsulfonyl)butanamide.

### EXAMPLES OF COMPOUNDS ACCORDING TO THE INVENTION

### Example 1

### (2R)—N-hydroxy-4-(6-((4-(hydroxymethyl)phenypethynyl)-2-oxobenzo[d]oxazol-3(2H)-yl)-2-methyl-2-(methylsulfonyl)butanamide

Starting from the compound of Preparation D (0.183 g; 0.34 mmol) and (4-ethynylphenyl)methanol (0.048 g; 0.36 mmol) and proceeding in analogy to Procedure D (81% yield) and Procedure B (53% yield), the racemate title compound (0.068 g) was obtained as a yellow solid. The latter was separated by mi-preparative chiral HPLC Method B (EtOH+1% TFA); flow rate: 16 mL/min, UV detection at 298 nm), the respective retention times (flow rate: 0.8 mL/min) were 6.4 and 8.7 min. The title enantiomer was identified as the second-eluting enantiomer and was obtained (0.022 g) as a yellow solid.

¹H NMR (d6-DMSO) δ: 11.05 (s, 1H), 9.27 (s, 1H), 7.57 (d, J=1.3 Hz, 1H), 7.52-7.45 (m, 3H), 7.36-7.31 (m, 3H), 5.30 (t, J=5.8 Hz, 1H), 4.53 (d, J=5.8 Hz, 2H), 3.99-3.89 (m, 1H), 3.75-3.65 (m, 1H), 3.04 (s, 3H), 2.72-2.62 (m, 1H), 2.12-2.01 (m, 1H), 1.61 (s, 3H).

MS (ESI, m/z): 459.1 [M+H⁺] for C₂₂H₂₂N₂O₇S; t_{R}=0.69 min.

### Example 2

### (2R)—N-hydroxy-4-(6-((1-(hydroxymethyl)cyclopropyl)buta4,3-diyn-1-yl)-2-oxobenzo[d]oxazol-3(2H)-yl)-2-methyl-2-(methylsulfonyl)butanamide

### 2.i. (2R)-4-(6-((1-(((tert-butyldiphenylsilyl)oxy)methyl)cyclopropyl)buta-1,3-diyn-1-yl)-2-oxobenzo[d]oxazol-3(2H)-yl)-2-methyl-2-(methylsulfonyl)-N-(((2RS)-tetrahydro-2H-pyran-2-yl)oxy)butanamide

Starting from the compound of Preparation E (0.150 g; 0.344 mmol) and ((1-(bromoethynyl)cyclopropyl)methoxy)(tert-butyl)diphenylsilane (prepared as described in WO 2005/036964, 0.191g; 0.46 mmol) and proceeding in analogy to Procedure F (68% yield), the title compound (0.180 g) was obtained after purification by CC (Hept-EA gradient) as a white solid.

MS (ESI, m/z): 769.08 [M+H⁺] for C₄₂H₄₈N₂O₈SSi; t_{R}=1.13 min.

### 2.ii. (2R)—N-hydroxy-4-(6((1-(hydroxymethyl)cyclopropyl)buta-1,3-diyn-1-yl)-2-oxobenzo[d]oxazol-3 (2H)-yl)-2-methyl-2-(methylsulfonyl)butanamide

Starting from the intermediate 2.i (0.18 g; 0.234 mmol) and proceeding successively in analogy to Preparation E, step E.iii (74% yield) and Procedure B (>95% yield), the title compound (0.055 g) was obtained after precipitation from water and drying to a constant weight, as a white solid.

¹H NMR (d6-DMSO) δ: 11.03 (s, 1H); 9.25 (s, 1H); 7.57 (d, J=1.3 Hz, 1H); 7.47 (dd, J=1.3, 8.1 Hz, 1H); 7.31 (d, J=8.1 Hz, 1H); 5.03 (t, J=6.1 Hz, 1H); 3.93 (m, 1H); 3.69 (m, 1H); 3.39 (d, J=6.1 Hz, 2H); 3.03 (s, 3H); 2.65 (m, 1H); 2.06 (m, 1H); 1.60 (s, 3H); 0.96-0.87 (m, 4H).

MS (ESI, m/z): 446.98.1 [M+H⁺] for C₂₁H₂₂N₂O₇; t_{R}=0.70 min.

### Example 3

### (2R)—N-hydroxy-2-methyl-2-(methylsulfonyl)-4-(6-((4-(morpholinomethyl)phenypethynyl)-2-oxobenzo[d]oxazol-3(2H)-yl)butanamide

Starting from the compound of Preparation E (0.161 g; 0.3 mmol) and 4-(4-ethynylbenzyl)morpholine (commercial, 0.062 g; 0.31 mmol) and proceeding successively in analogy to Procedure D (92% yield) and Procedure E (50% yield), the title compound was obtained as formate salt, after purification by prep-HPLC, as a yellowish solid (0.080 g).

MS (ESI, m/z): 528.1 [M+H⁺] for C₂₇H₃₁N₃O₉S; t_{R}=0.58 min.

### Example 4

### (2R)—N-hydroxy-2-methyl-4-(6-((1-(methylamino)cyclopropyl)buta-1,3-diyn-1-yl)-2-oxobenzo[d]oxazol-3(2H)-yl)-2-(methylsulfonyl)butanamide

Starting from the compound of Preparation E (0.150 g; 0.34 mmol) and the compound of Preparation G (0.097 g; 0.46 mmol) and proceeding in analogy to Procedure F (65% yield) and Procedure G (60% yield), the title compound (0.068 g) was obtained, after purification by prep-HPLC (Method 2), as a yellowish solid.

¹H NMR (d6-DMSO) δ: 11.11-10.90 (br. s, 1H); 9.32-9.19 (br. s, 1H); 7.58 (d, J=1.3 Hz, 1H); 7.48 (dd, J=1.4, 8.1 Hz, 1H); 7.31 (d, J=8.1 Hz, 1H); 3.93 (m, 1H); 3.70 (m, 1H); 3.03 (s, 3H); 2.65 (m, 1H); 2.34 (s, 3H); 2.06 (m, 1H); 1.60 (s, 3H); 1.00-0.94 (m, 2H); 0.90-0.84 (m, 2H).

MS (ESI, m/z): 446.0 [M+H⁺] for C₂₁H₂₃N₃O₆S; t_{R}=0.55 min.

### Example 5

### (2R)—N-hydroxy-4-(6-(((1S,2S)-2-(hydroxymethyl)cyclopropyl)buta-1,3-diyn-1-yl)-2-oxobenzo[d]oxazol-3 (2H)-yl)-2-methyl-2-(methylsulfonyl)butanamide

Starting from the compound of Preparation E (0.118 g; 0.23 mmol) and the compound of Preparation H (0.048 g; 0.46 mmol) and proceeding in analogy to Procedure F (66% yield) and Procedure B (98% yield), the title compound (0.054 g) was obtained, after washing with Et₂O, as a white solid.

¹H NMR (d6-DMSO) δ: 11.03 (br. s., 1H); 9.25 (br. s., 1H); 7.56 (s, 1H); 7.46 (d, J=8.2 Hz, 1H); 7.30 (d, J=8.2 Hz, 1H); 4.71 (t, J=5.7 Hz, 1H); 3.92 (m, 1H); 3.68 (m, 1H); 3.42 (m, 1H); 3.25 (m, 1H); 3.02 (s, 3H); 2.65 (m, 1H); 2.05 (m, 1H); 1.59 (s, 3H); 1.47-1.37 (m, 2H); 0.95-0.83 (m, 2H).

MS (ESI, m/z): 447.0 [M+H⁺] for C₂₁H₂₂N₂O₇S; t_{R}=0.69 min.

### Example 6

### (2R)-4-(6-(((1R,2R)-2-fluoro-2-(hydroxymethyl)cyclopropyl)buta-1,3-diyn-1-yl)-2-oxobenzo[d]oxazol-3(2H)-yl)-N-hydroxy-2-methyl-2-(methylsulfonyl)butanamide

### 6.i. ((1R,2R)-1-fluoro-2-((3-((3R)-3-methyl-3-(methylsulfonyl)-4-oxo-4-((((R)-tetrahydro-2H-pyran-2-yl)oxy)amino)butyl)-2-oxo-2,3-dihydrobenzo[d]oxazol-6-yl)buta-1,3-diyn-1-yl)cyclopropyl)methyl benzoate

Starting from the compound of Preparation E (0.118 g; 0.23 mmol) and the compound of Preparation I (0.082 g; 0.27 mmol) and proceeding in analogy to Procedure F (81% yield), the title compound (0.121 g) was obtained, after purification by CC (Hept-EA-MeOH), as a white solid.

¹H NMR (d6-DMSO) δ: (mixture of diastereomers) 11.44 (br. s, 1H); 8.03-8.00 (m, 2H); 7.70 (m, 1H); 7.61-7.55 (m, 3H); 7.50 (m, 1H); 7.30 (t, J=8.5 Hz, 1H); 4.92 (m, 0.5H); 4.85 (m, 0.5H); 4.73-4.52 (m, 2H); 4.10 (m, 0.5H); 4.02 (m, 0.5H); 3.92 (m, 1H); 3.78 (m, 0.5H); 3.70 (m, 0.5H); 3.48 (m, 1H); 3.04 (s, 1.5H); 3.02 (s, 1.5H); 2.65 (m, 1H); 2.29 (m, 1H); 2.08 (m, 1H); 1.71-1.49 (m, 11H).

MS (ESI, m/z): 652.99 [M+H⁺] for C₃₃H₃₃N₂O₉FS; t_{R}=0.99 min.

### 6.ii. (2R)-4-(6-(((1R, 2R)-2-fluoro-2-(hydroxymethyl)cyclopropyl)buta-1,3-diyn-1-yl)-2-oxobenzo[d]oxazol-3(2H)-yl)-N-hydroxy-2-methyl-2-(methylsulfonyl)butanamide

Starting from intermediate 6.i (0.12 g; 0.18 mmol) and proceeding successively in analogy to Preparation H, step H.iii (33% yield) and Procedure B (42% yield), the title compound (0.010 g) was obtained, after trituration in Et₂O, as a white solid.

¹H NMR (d6-DMSO) δ: 11.03 (br. s., 1H); 9.24 (br. s., 1H); 7.59 (s, 1H); 7.49 (d, J=8.2 Hz, 1H); 7.31 (d, J=8.2 Hz, 1H); 5.24 (d, J=6.1 Hz, 1H); 3.92 (m, 1H); 3.74-3.56 (m, 3H); 3.02 (s, 3H); 2.65 (m, 1H); 2.05 (m, 1H); 1.96 (m, 1H); 1.59 (s, 3H); 1.43-1.31 (m, 2H).

MS (ESI, m/z): 464.99 [M+H⁺] for C₂₁H₂₁N₂O₇FS; t_{R}=0.69 min.

### Example 7

### (3R)-5-(3-(4-(hydroxyamino)-3-methyl-3-(methylsulfonyl)-4-oxobutyl)-2-oxo-2,3-dihydrobenzo[d]oxazol-6-yl)penta-2,4-diyn-1-yl 3-hydroxyazetidine-1-carboxylate

Starting from the compound of Preparation E (0.100 g; 0.19 mmol) and the compound of Preparation J (0.064 g; 0.27 mmol) and proceeding in analogy to Procedure F (87% yield) and Procedure B (72% yield), the title compound (0.054 g) was obtained, after washing with Et₂O, as a white solid.

¹H NMR (d6-DMSO) δ: 11.03 (br. s, 1H); 9.25 (br. s, 1H); 7.64 (m, 1H); 7.53 (dd, J=1.4, 8.1 Hz, 1H); 7.33 (m, 1H); 5.74 (d, J=6.5 Hz, 1H); 4.84 (s, 2H); 4.44 (m, 1H); 4.06-4.19 (m, 2H); 3.93 (m, 1H); 3.64-3.74 (m, 3H); 3.02 (s, 3H); 2.65 (m, 1H); 2.06 (m, 1H); 1.59 (s, 3H).

MS (ESI, m/z): 505.99 [M+H⁺] for C₂₃H₂₃N₃O₉S; t_{R}=0.67 min.

### Example 8

### (2R)-4-(6-((1-aminocyclopropyl)buta-1,3-diyn-1-yl)-2-oxobenzo[d]oxazol-3(2H)-yl)-N-hydroxy-2-methyl-2-(methylsulfonyl)butanamide

Starting from the compound of Preparation E (0.120 g; 0.23 mmol) and the compound of Preparation F (0.064 g; 0.33 mmol) and proceeding in analogy to Procedure F (82% yield) and Procedure B (41% yield), the title compound (0.034 g) was obtained, after purification by CC (DCM-MeOH), as a beige solid.

¹H NMR (d6-DMSO) δ: 10.85-11.18 (br. s, 1H); 9.32-9.12 (br. s, 1H); 7.56 (d, J=1.4 Hz, 1H); 7.46 (m, 1H); 7.30 (d, J=8.2 Hz, 1H); 3.92 (m, 1H); 3.69 (m, 1H); 3.02 (s, 3H); 2.65 (m, 1H); 2.05 (m, 1H); 1.59 (s, 3H); 0.98-0.93 (m, 2H); 0.88-0.82 (m, 2H).

MS (ESI, m/z): 473.02 [M+H⁺] for C₂₀H₂₁N₃O₆S; t_{R}=0.53 min.

### Example 9

### (2R)—N-hydroxy-2-methyl-4-(6-((1-methylazetidin-3-yl)buta-1,3-diyn-1-yl)-2-oxobenzo[d]oxazol-3(2H)-yl)-2-(methylsulfonyl)butanamide

### 9.i. (2R)-4-(6-(azetidin-3-ylbuta-1,3-diyn-1-yl)-2-oxobenzo[d]oxazol-3(2H)-yl)-2-methyl-2-(methylsulfonyl)-N-(((2RS)-tetrahydro-2H-pyran-2-yl)oxy)butanamide

Starting from the compound of Preparation E (0.175 g; 0.34 mmol) and the compound of Preparation K (0.091 g; 0.46 mmol) and proceeding in analogy to Procedure F (65% yield), the title compound (0.14 g) was obtained, after purification by CC (DCM-MeOH), as a white solid.

¹H NMR (d6-DMSO) δ: 7.58 (m, 1H); 7.49 (m, 1H); 7.31 (m, 1H); 4.91 (m, 0.5H); 4.86 (m, 0.5H); 4.09 (m, 0.5H); 4.01 (m, 0.5H); 3.92 (m, 1H); 3.79 (m, 0.5H); 3.73 (m, 0.5H); 3.66-3.45 (m, 6H); 3.04 (s, 1.5H); 3.02 (s, 1.5H); 2.63 (m, 1H); 2.07 (m, 1H); 1.72-1.62 (m, 3H); 1.62-1.58 (m, 3H); 1.53 (m, 3H).

MS (ESI, m/z): 557.11 [M+MeCN+H⁺] for C₂₅H₂₉N₃O₇S; t_{R}=0.63 min.

### 9.ii. (2R)-2-methyl-4-(6-((1-methylazetidin-3-yl)buta-1,3-diyn-1-yl)-2-oxobenzo[d]oxazol-3(2H)-yl)-2-(methylsulfonyl)-N-(((2RS)-tetrahydro-2H-pyran-2-yl)oxy)butanamide

To a mixture of intermediate 9.i (0.110 g; 0.213 mmol) in DCM (2.7 mL) were added 37% aq. formaldehyde (0.05 mL, 0.641 mmol) and NaBH(OAc)3 (0.28 g, 1.28 mmol). The reaction mixture was stirred for 30 min. Sat. NaHCO₃ (10 mL) and DCM (10 mL) were added. The aq. layer was extracted with DCM-MeOH (9-1, 3×10 mL). The evaporation residue was purified by CC (DCM-MeOH) to afford the title compound (0.0934 g, 83% yield) as a white foam.

¹H NMR (d6-DMSO) δ: 11.42 (br s, 1H); 7.58 (m, 1H); 7.49 (m, 1H); 7.30 (m, 1H); 4.91 (m, 0.5H); 4.86 (m, 0.5H); 4.09 (m, 0.5H); 4.01 (m, 0.5H); 3.92 (m, 1H); 3.78 (m, 0.5H); 3.71 (m, 0.5H); 3.54-3.44 (m, 3H); 3.36 (m, 1H); 3.04 (s, 1.5H); 3.02 (s, 1.5H); 2.99 (t, J=6.7 Hz, 2H); 2.65 (m, 1H); 2.19 (s, 3H); 2.08 (m, 1H); 1.72-1.64 (m, 3H); 1.62 (s, 1.5H); 1.61 (s, 1.5H); 1.57-1.49 (m, 3H).

MS (ESI, m/z): 571.08 [M+MeCN+H⁺] for C₂₆H₃₁N₃O₇S; t_{R}=0.64 min.

### (2R)—N-hydroxy-2-methyl-4-(6-((1-methylazetidin-3-yl)buta-1,3-diyn-1-yl)-2-oxobenzo[d]oxazol-3(2H)-yl)-2-(methylsulfonyl)butanamide

Starting from intermediate 9.ii (0.093 g; 0.17 mmol) and proceeding in analogy to Procedure B (32% yield), the title compound (0.025 g) was obtained, after purification by CC (DCM-MeOH containing 1% aq. NH₄OH), as a white solid.

¹H NMR (d6-DMSO) δ: 11.01 (br. s, 1H); 9.26 (br. s, 1H); 7.59 (d, J=1.4 Hz, 1H); 7.49 (m, 1H); 7.31 (d, J=8.2 Hz, 1H); 3.93 (m, 1H); 3.69 (m, 1H); 3.50 (t, J=7.4 Hz, 2H); 3.37 (m, 1H); 3.02 (s, 3H); 3.00 (t, J=6.9 Hz, 2H); 2.64 (m, 1H); 2.19 (s, 3H); 2.06 (m, 1H); 1.59 (s, 3H).

MS (ESI, m/z): 446.0 [M+MeCN+H₊] for C₂₁H₂₃N₃O₆S; t_{R}=0.53 min.

### Example 10

### (2R)—N-hydroxy-2-methyl-4-(6-((1-methylazetidin-3-yl)buta-1,3-diyn-1-yl)-2-oxobenzo[d]thiazol-3(2H)-yl)-2-(methylsulfonyl)butanamide

Starting from the compound of Preparation L (0.2 g; 0.376 mmol) and the compound of Preparation K (0.107 g; 0.54 mmol) and proceeding in analogy to Procedure F (68% yield), Example 9, step 9.ii (86% yield) and Procedure G (% yield), the title compound (0.14 g) was obtained, after purification by prep-HPLC (Method 2), as a white solid.

¹H NMR (d6-DMSO) δ: 11.02 (br. s, 1H); 9.30 (br. s, 1H); 7.93 (d, J=1.5 Hz, 1H); 7.61 (dd, J=1.6, 8.4 Hz, 1H); 7.37 (d, J=8.5 Hz, 1H); 4.09 (m, 1H); 3.74 (m, 1H); 3.49 (t, J=7.3 Hz, 2H); 3.36 (m, 1H); 3.02 (s, 3H); 3.00 (t, J=6.8 Hz, 2H); 2.56 (m, 1H); 2.19 (s, 3H); 1.96 (m, 1H); 1.61 (s, 3H).

MS (ESI, m/z): 503.00 [M+MeCN+H⁺] for C₂₁H₂₃N₃O₅S₂; t_{R}=0.56 min.

### Example 11

### (2R)-4-(6-(((1R,2R)-2-fluoro-2-(hydroxymethyl)cyclopropyl)buta-1,3-diyn-1-yl)-2-oxobenzo[d]thiazol-3(2H)-yl)-N-hydroxy-2-methyl-2-(methylsulfonyl)butanamide

Starting from the compound of Preparation L (0.2 g; 0.376 mmol) and the compound of Preparation M (0.107 g; 0.54 mmol) and proceeding in analogy to Procedure F (68% yield) and Procedure B (% yield), the title compound (0.14 g) was obtained, after trituration in Et₂O, as a white solid.

¹H NMR (d6-DMSO) δ: 7.58 (m, 1H); 7.49 (m, 1H); 7.31 (m, 1H); 4.91 (m, 0.5H); 4.86 (m, 0.5H); 4.09 (m, 0.5H); 4.01 (m, 0.5H); 3.92 (m, 1H); 3.79 (m, 0.5H); 3.73 (m, 0.5H); 3.66-3.45 (m, 6H); 3.04 (s, 1.5H); 3.02 (s, 1.5H); 2.63 (m, 1H); 2.07 (m, 1H); 1.72-1.62 (m, 3H); 1.62-1.58 (m, 3H); 1.53 (m, 3H).

MS (ESI, m/z): 480.94 [M+H⁺] for C₂₁H₂₁N₂O₆FS₂; t_{R}=0.72 min.

### Example 12

### ((1S,2S)-2-((3-((3R)-4-(hydroxyamino)-3-methyl-3-(methylsulfonyl)-4-oxobutyl)-2-oxo-2,3-dihydrobenzo[d]oxazol-6-yl)buta-1,3-diyn-1-yl)cyclopropyl)methyl dihydrogen phosphate

Starting from the compound of Preparation E (0.130 g; 0.25 mmol) and the compound of Preparation N (0.150 g; 0.41 mmol) and proceeding in analogy to Procedure F (89% yield) and Procedure H (54% yield), the title compound (0.121 g) was obtained, after purification by prep-HPLC (Method 3), as a white solid.

¹H NMR (d6-DMSO) δ: 11.01 (br. s, 1H); 9.23 (br. s, 1H); 7.56 (d, J=1.3 Hz, 1H); 7.46 (dd, J=1.3, 8.1 Hz, 1H); 7.30 (d, J=8.1 Hz, 1H); 3.92 (m, 1H); 3.80 (m, 1H); 3.74-3.58 (m, 2H); 3.02 (s, 3H); 2.65 (m, 1H); 2.05 (m, 1H); 1.63-1.56 (m, 5H); 1.03 (m, 1H); 0.96 (m, 1H).

MS (ESI, m/z): 526.96 [M+H⁺] for C₂₁H₂₃N₂O₁₀PS; t_{R}=0.58 min.

### Example 13

### (2R)—N-hydroxy-4-(6-((4-(hydroxymethyl)phenypethynyl)-2-oxobenzo[d]thiazol-3(2H)-yl)-2-methyl-2-(methylsulfonyl)butanamide

Starting from intermediate L.iv (0.197 g; 0.36 mmol) and (4-ethynylphenyl)methanol (0.052 g; 0.39 mmol) and proceeding in analogy to Procedure D (81% yield) and Procedure B (53% yield), the title compound (0.071 g) was obtained, after purification by prep-HPLC (Method 1) as a beige solid.

¹H NMR (d6-DMSO) δ: 11.06 (s, 1H); 9.31 (s, 1H); 7.93 (d, J=1.5 Hz, 1H); 7.60 (dd, J=1.5, 8.4 Hz, 1H); 7.51 (d, J=8.1 Hz, 2H); 7.40-7.35 (m, 3H); 5.30 (t, J=5.8 Hz, 1H); 4.53 (d, J=5.8 Hz, 2H); 4.14-4.05 (m, 1H); 3.80-3.71 (m, 1H); 3.04 (s, 3H); 2.65-2.46 (overlapped m, 1H); 2.03-1.93 (m, 1H); 1.62 (s, 3H).

MS (ESI, m/z): 475.93 [M+H⁺] for C₂₂H₂₂N₂O₆S₂; t_{R}=0.72 min.

### Pharmacological Properties of the Invention Compounds

### In Vitro Assays

### Bacterial Growth Minimal Inhibitory Concentrations:

### Experimental Methods:

Minimal Inhibitory Concentrations (MICs; mg/L) were determined in cation-adjusted MuellerHinton Broth by a microdilution method following the description given in “Methods for Dilution Antimicrobial Susceptibility Tests for Bacteria that grow Aerobically”, Approved standard, 7th ed., Clinical and Laboratory Standards Institute (CLSI) Document M7-A7, Wayne, Pa., USA (2006).

### Results:

All Example compounds were tested against several Gram-positive and Gram-negative bacteria. Typical antibacterial test results are given in Table 1 hereafter (MICs in mg/L). K pneumoniae A-651 is a multiply-resistant strain (in particular quinolone-resistant), while E. coli ATCC25922 and P. aeruginosa ATCC27853 are quinolone-sensitive strains.

TABLE 1

| | MIC for | MIC for | MIC for |
|---|---|---|---|
| Example | E. coli | P. aeruginosa | K. Pneumoniae |
| No. | ATCC25922 | ATCC27853 | A-651 |
| RE1 | 2 | >8 | 1 |
| RE2 | 4 | >8 | 8 |
| 1 | 0.25 | 4 | 0.5 |
| 2 | 0.25 | 1 | 1 |
| 3 | 2 | >8 | 2 |
| 4 | 0.25 | 1 | 0.25 |
| 5 | 0.063 | 0.5 | 0.125 |
| 6 | 0.125 | 0.5 | 0.25 |
| 7 | 0.5 | 1 | 1 |
| 8 | 0.125 | 1 | 0.25 |
| 9 | 1 | 1 | 0.5 |
| 10 | 0.5 | 2 | 1 |
| 11 | 0.063 | 1 | 0.25 |
| 13 | 0.125 | 8 | 0.5 |
| Cipro | ≤0.063 | 0.125 | >8 |

The compounds of Examples 5 and 12 were tested against wild-type E. coli A-1261 in the absence of alkaline phosphatase or esterase, in the presence of an alkaline phosphatase, and in the presence of an esterase. The corresponding antibacterial test results are given in Table 2 hereafter (MICs in mg/L).

TABLE 2

MIC for E. coli A-1261

| | In the absence | In the presence | |
|---|---|---|---|
| | of alkaline | of an alkaline | In the presence |
| Example | phosphatase | phosphatase | of an esterase |
| No. | or esterase | (2 i.U./mL) | (10 i.U./mL) |
| 12 | >8 | 0.125 | >8 |
| 5 | 0.063 | 0.063 | 0.063 |

1. A compound of formula I 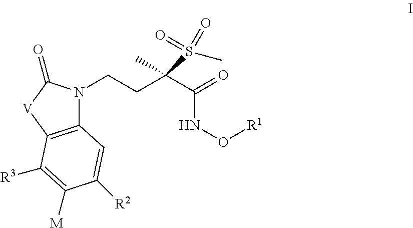
wherein
V represents O or S;
R² and R³ each independently represent hydrogen or fluorine; and
M is one of the groups M^{A} and M^{B} represented below 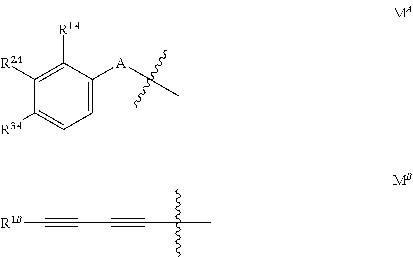
wherein in group M^{A}:
A represents a bond or —C≡C—;
R^{1A} represents hydrogen, or halogen;
R^{2A} represents hydrogen, or halogen; and
R^{3A} represents
(C₁-C₃)alkoxy;
hydroxy(C₁-C₄)alkyl; or
R^{NA1}R^{NA2}N—(C₁-C₃)alkyl, wherein R^{NA1} and R^{NA2} independently represent hydrogen or (C₁-C₃)alkyl; or R^{NA1} and R^{NA2} together with the nitrogen to which they are attached form a 4- to 6-membered saturated ring optionally containing one oxygen ring atom, wherein said ring is unsubstituted, or mono-substituted with fluoro; and
wherein in group M^{B}:
R^{1B} represents
hydroxy(C₁-C₄)alkyl;
1-(3-hydroxyazetidine)-1-carbonyloxymethyl;
1-hydroxymethyl-cycloprop-1-yl, or a group which is a prodrug thereof wherein said group is 1-((phosphonooxy)methyl)-cycloprop-1-yl, 1-[(di(C₁-C₄)alkylamino)-(C₁-C₃)alkyl-carbonyloxymethyl]-cycloprop-1-yl, 1-{[(2-(phosphonooxy-(C₁-C₄)alkyl)-phenyl)-(C₁-C₄)alkyl]-carbonyloxymethyl}-cycloprop-1-yl, 1-{[2-(phosphonooxy-(C₁-C₄)alkyl)-phenyl]-carbonyloxymethyl}-cycloprop-1-yl, or 1-{[(2-phosphonooxy-phenyl)-(C₁-C₄)alkyl]-carbonyloxymethyl}-cycloprop-1-yl;
trans-2-hydroxymethyl-cycloprop-1-yl, or a group which is a prodrug thereof wherein said group is trans-2-(phosphonooxymethyl)-cycloprop-1-yl, trans-2-[(di(C₁-C₄)alkylamino)-(C₁-C₃)alkyl-carbonyloxymethyl]-cycloprop-1-yl, trans-2-{[(2-(phosphonooxy-(C₁-C₄)alkyl)-phenyl)-(C₁-C₄)alkyl]-carbonyloxymethyl}-cycloprop-1-yl, trans-2-{[2-(phosphonooxy-(C₁-C₄)alkyl)-phenyl]-carbonyloxymethyl}-cycloprop-1-yl, or trans-2-{[(2-phosphonooxy-phenyl)-(C₁-C₄)alkyl]-carbonyloxymethyl}-cycloprop-1-yl;
2-fluoro-2-hydroxymethyl-cycloprop-1-yl, or a group which is a prodrug thereof wherein said group is 2-fluoro-2-(phosphonooxymethyl)-cycloprop-1-yl, 2-fluoro-2-[(di(C₁-C₄)alkylamino)-(C₁-C₃)alkyl-carbonyloxymethyl]-cycloprop-1-yl, 2-fluoro-2-{[(2-(phosphonooxy-(C₁-C₄)alkyl)-phenyl)-(C₁-C₄)alkyl]-carbonyloxymethyl}-cycloprop-1-yl, 2-fluoro-2-{[2-(phosphonooxy-(C₁-C₄)alkyl)-phenyl]-carbonyloxymethyl}-cycloprop-1-yl, or 2-fluoro-2-{[(2-phosphonooxy-phenyl)-(C₁-C₄)alkyl]-carbonyloxymethyl}-cycloprop-1-yl;
3-hydroxy-oxetan-3-yl;
trans-2-(1,2-dihydroxyethyl)cycloprop-1-yl;
trans-(cis-3,4-dihydroxy)-cyclopent-1-yl;
R^{NB1}R^{NB2}N—(C₁-C₃)alkyl, wherein R^{NB1} and R^{NB2} independently represent hydrogen or (C₁-C₃)alkyl;
1-(R^{NB3}R^{NB4}N)-cycloprop-1-yl, wherein R^{NB3} and R^{NB4} independently represent hydrogen or (C₁-C₃)alkyl, or R^{NB3} represents hydrogen and R^{NB4} (phosphonooxy)methoxy-carbonyl;
azetidin-3-yl, wherein said azetidin-3-yl is unsubstituted, or mono- or di-substituted wherein, if present,
one substituent is attached in position 1 of said azetidin-3-yl group, wherein said substituent is selected from (C₁-C₃)alkyl, (C₃-C₄)cycloalkyl, oxetan-3-yl, ω-fluoro-(C₂)alkyl, or ω-hydroxy-(C₂-C₄)alkyl; and/or
one substituent is fluoro attached in position 3 of said the azetidin-3-yl group;
R¹ represents hydrogen, or R¹ represents —PO₃H₂, —SO₃H, phosphonooxymethyl, or the group L represented below 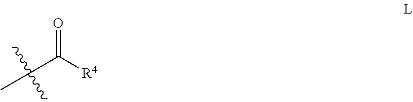
wherein R⁴ represents (C₁-C₄)alkylamino(C₁-C₄)alkyl, di(C₁-C₄)alkylamino(C₁-C₄)alkyl, phosphonooxy(C₁-C₄)alkyl, phosphonooxymethoxy, 2-(phosphonooxy-(C₁-C₄)alkyl)-phenyl, [2-(phosphonooxy-(C₁-C₄)alkyl)-phenyl]-(C₁-C₄)alkyl, or (2-(phosphonooxy)-phenyl)-(C₁-C₄)alkyl;
or a pharmaceutically acceptable salt thereof.

2. A compound according to claim 1, wherein V represents O;
or a pharmaceutically acceptable salt thereof.

3. A compound according to claim 1 wherein R² and R³ both represent hydrogen;
or a pharmaceutically acceptable salt thereof.

4. A compound according to claim 1, wherein R¹ represents hydrogen, or R¹ represents —PO₃H₂, —SO₃H, phosphonooxymethyl, or dimethylaminomethylcarbonyl;
or a pharmaceutically acceptable salt thereof.

5. A compound according to claim 1, wherein R¹ represents hydrogen;
or a pharmaceutically acceptable salt thereof.

6. A compound according to claim 1, wherein M is one of the groups M^{A} and M^{B} represented below 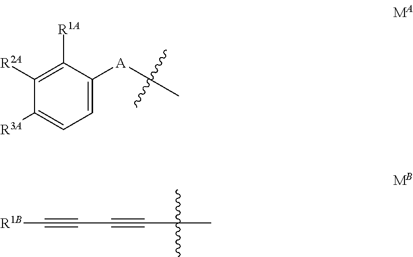
wherein in group M^{A}:
A represents a bond;
R^{1A} represents hydrogen or fluoro;
R^{2A} represents hydrogen; and
R^{3A} represents (C₁-C₃)alkoxy; or
A represents —C≡C—;
R^{1A} represents hydrogen;
R^{2A} represents hydrogen; and
R^{3A} represents
hydroxy(C₁-C₄)alkyl; or
R^{NA1}R^{NA2}N—(C₁-C₃)alkyl, wherein R^{NA1} and R^{NA2} independently represent hydrogen or (C₁-C₃)alkyl; or R^{NA1} and R^{NA2} together with the nitrogen to which they are attached form a 4- to 6-membered saturated ring optionally containing one oxygen ring atom, wherein said ring is unsubstituted, or mono-substituted with fluoro;
wherein in group M^{B}:
R^{1B} represents
hydroxy(C₁-C₄)alkyl;
1-(3-hydroxyazetidine)-1-carbonyloxymethyl;
1-hydroxymethyl-cycloprop-1-yl, or a group which is a prodrug thereof wherein said group is 1-((phosphonooxy)methyl)-cycloprop-1-yl, or 1-[(di(C₁-C₄)alkylamino)-(C₁-C₃)alkyl-carbonyloxymethyl]-cycloprop-1-yl;
trans-2-hydroxymethyl-cycloprop-1-yl, or a group which is a prodrug thereof wherein said group is trans-2-(phosphonooxymethyl)-cycloprop-1-yl, or trans-2-[(di(C₁-C₄)alkylamino)-(C₁-C₃)alkyl-carbonyloxymethyl]-cycloprop-1-yl;
2-fluoro-2-hydroxymethyl-cycloprop-1-yl, or a group which is a prodrug thereof wherein said group is 2-fluoro-2-(phosphonooxymethyl)-cycloprop-1-yl;
3-hydroxy-oxetan-3-yl;
trans-2-(1 ,2-dihydroxyethyl)cycloprop-1-yl;
trans-(cis-3,4-dihydroxy)-cyclopent-1-yl;
R^{NB1}R^{NB2}N—(C₁-C₃)alkyl, wherein R^{NB1} and R^{NB2} independently represent hydrogen or (C₁-C₃)alkyl;
1-(R^{NB3}R^{NB4}N)-cycloprop-1-yl, wherein R^{NB3} and R^{NB4} independently represent hydrogen or (C₁-C₃)alkyl, or R^{NB3} represents hydrogen and R^{NB4} (phosphonooxy)methoxy-carbonyl;
azetidin-3-yl, wherein said azetidin-3-yl is mono-or di-substituted wherein
one substituent is attached in position 1 of said azetidin-3-yl group, wherein said substituent is selected from (C₁-C₃)alkyl, (C₃-C₄)cycloalkyl, oxetan-3-yl, ω-fluoro-(C₂)alkyl, or ω-hydroxy-(C₂-C₄)alkyl; and, if present,
one substituent is fluoro attached in position 3 of said the azetidin-3-yl group;
or a pharmaceutically acceptable salt thereof.

7. A compound according to claim 1, wherein M is one of the groups M^{A} and M^{B} represented below 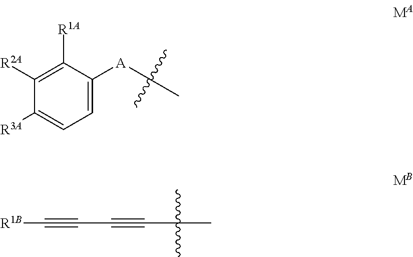
wherein in group M^{A}:
A represents —C≡C—;
R^{1A} represents hydrogen;
R^{2A} represents hydrogen; and
R^{3A} represents hydroxymethyl; and
wherein in group M^{B}:
R^{1B} represents
1-(3-hydroxyazetidine)-1-carbonyloxymethyl;
1-hydroxymethyl-cycloprop-1-yl;
trans-2-hydroxymethyl-cycloprop-1-yl, or a group which is a prodrug thereof wherein said group is trans-2-(phosphonooxymethyl)-cycloprop-1-yl;
cis-2-fluoro-2-hydroxymethyl-cycloprop-1-yl;
1-aminocycloprop-1-yl or 1-(methylamino)cycloprop-1-yl;
N-methylazetidin-3-yl, N-ethylazetidin-3-yl, N-isopropylazetidin-3-yl, N-cyclopropylazetidin-3-yl, 3-fluoro-1-methyl-azetidin-3-yl, 3-fluoro-1-ethyl-azetidin-3-yl, 3-fluoro-1-isopropyl-azetidin-3-yl, or 3-fluoro-1-cyclopropyl-azetidin-3-yl;
or a pharmaceutically acceptable salt thereof.

8. A compound according to claim 1, wherein M is the group M^{B};
or a pharmaceutically acceptable salt thereof.

9. A compound of formula I according to claim 1, which is selected from the following:
(R)-4-(6-(2-fluoro-4-methoxyphenyl)-2-oxobenzo[d]oxazol-3(2H)-yl)-N-hydroxy-2-methyl-2-(methylsulfonyl)butanamide;
(R)—N-hydroxy-4-(6-((3-hydroxyoxetan-3-yl)buta-1,3-diyn-1-yl)-2-oxobenzo[d]oxazol-3(2H)-yl)-2-methyl-2-(methylsulfonyl)butanamide;
(R)—N-hydroxy-4-(6-((4-(hydroxymethyl)phenypethynyl)-2-oxobenzo[d]oxazol-3(2H)-yl)-2-methyl-2-(methylsulfonyl)butanamide;
(R)—N-hydroxy-4-(6-((1-(hydroxymethyl)cyclopropyl)buta-1,3-diyn-1-yl)-2-oxobenzo[d]oxazol-3(2H)-yl)-2-methyl-2-(methylsulfonyl)butanamide;
(R)—N-hydroxy-2-methyl-2-(methylsulfonyl)-4-(6-((4-(morpholinomethyl)phenyl)ethynyl)-2-oxobenzo[d]oxazol-3(2H)-yl)butanamide;
(R)—N-hydroxy-2-methyl-4-(6-((1-(methylamino)cyclopropyl)buta-1,3-diyn-1-yl)-2-oxobenzo[d]oxazol-3(2H)-yl)-2-(methylsulfonyl)butanamide;
(R)—N-hydroxy-4-(6-(((1S,2S)-2-(hydroxymethyl)cyclopropyl)buta-1,3-diyn-1-yl)-2-oxobenzo[d]oxazol-3(2H)-yl)-2-methyl-2-(methylsulfonyl)butanamide;
(R)-4-(6-(((1R,2R)-2-fluoro-2-(hydroxymethyl)cyclopropyl)buta-1,3-diyn-1-yl)-2-oxobenzo[d]oxazol-3(2H)-yl)-N-hydroxy-2-methyl-2-(methylsulfonyl)butanamide;
(R)-5-(3-(4-(hydroxyamino)-3-methyl-3-(methylsulfonyl)-4-oxobutyl)-2-oxo-2,3-dihydrobenzo[d]oxazol-6-yl)penta-2,4-diyn-1-yl 3-hydroxyazetidine-1-carboxylate;
(R)-4-(6-((1-am inocyclopropyl)buta-1, 3-d iyn-1-yl)-2-oxobenzo[d]oxazol-3(2H)-yl)-N-hydroxy-2-methyl-2-(methylsulfonyl)butanamide;
(R)—N-hydroxy-2-methyl-4-(6-((1-methylazetidin-3-yl)buta-1,3-diyn-1-yl)-2-oxobenzo[d]oxazol-3(2H)-yl)-2-(methylsulfonyl)butanamide;
(R)—N-hydroxy-2-methyl-4-(6-((1-methylazetidin-3-yl)buta-1,3-diyn-1-yl)-2-oxobenzo[d]thiazol-3(2H)-yl)-2-(methylsulfonyl)butanamide;
(R)-4-(6-(((1R,2R)-2-fluoro-2-(hydroxymethyl)cyclopropyl)buta-1,3-diyn-1-yl)-2-oxobenzo[d]thiazol-3(2H)-yl)-N-hydroxy-2-methyl-2-(methylsulfonyl)butanamide;
((1S,2S)-2-((3-((R)-4-(hydroxyamino)-3-methyl-3-(methylsulfonyl)-4-oxobutyl)-2-oxo-2,3-dihydrobenzo[d]oxazol-6-yl)buta-1,3-diyn-1-yl)cyclopropyl)methyl dihydrogen phosphate; and
(R)—N-hydroxy-4-(6-((4-(hydroxymethyl)phenypethynyl)-2-oxobenzo[d]thiazol-3(2H)-yl)-2-methyl-2-(methylsulfonyl)butanamide;
or a pharmaceutically acceptable salt thereof.

10. As a medicament, a compound of formula I as defined in claim 1, or a pharmaceutically acceptable salt thereof.

11. A pharmaceutical composition containing, as active ingredient, a compound of formula I as defined in claim 1, or a pharmaceutically acceptable salt thereof, and at least one therapeutically inert excipient.

12. A compound of formula I as defined in claim 1, or a pharmaceutically acceptable salt thereof, for the prevention or treatment of a bacterial infection.

13. A compound or pharmaceutically acceptable salt according to claim 12, wherein said bacterial infection is a bacterial infection caused by Gram-negative bacteria.
